SEQUENCE LISTING (12 sequences)

SEQ ID NO: 1 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 10 aa, synthetic construct
GFTFSDSWIH

SEQ ID NO: 4 - protein, 18 aa, synthetic construct
AWISPYGGSTYYADSVKG

SEQ ID NO: 5 - protein, 9 aa, synthetic construct
RHWPGGFDY

SEQ ID NO: 6 - protein, 11 aa, synthetic construct
RASQDVSTAVA

SEQ ID NO: 7 - protein, 7 aa, synthetic construct
SASFLYS

SEQ ID NO: 8 - protein, 9 aa, synthetic construct
QQYLYHPAT

SEQ ID NO: 9 - protein, 118 aa, synthetic construct [sequence omitted]

SEQ ID NO: 10 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 11 - protein, 115 aa, synthetic construct
QSLEESGGRLVKPDETLTITCTVSGIDLSSNGLTWVRQAPGEGLEWIGTINKDASAYYASWAKGRLTISKPSSTKVDLKITSPTTEDTATYFCGRIAFKTGTSIWGPGTLVTVSS

SEQ ID NO: 12 - protein, 112 aa, synthetic construct
AIVMTQTPSPVSAAVGGTVTINCQASESVYSNNYLSWFQQKPGQPPKLLIYLASTLASGVPSRFKGSGSGTQFTLTISGVQCDDAATYYCIGGKSSSTDGNAFGGGTEVVVR

(19) United States
(12) Patent Application Publication
(10) Pub. No.: US 2025/0257134 A1
(43) Pub. Date: Aug. 14, 2025

(54) THERAPEUTIC AND DIAGNOSTIC METHODS FOR BLADDER CANCER

(71) Applicant: Genentech, Inc., South San Francisco, CA (US)

(72) Inventors: Sanjeev MARIATHASAN, Millbrae, CA (US); Maureen Frances HARRYMAN, Petaluma, CA (US); Romain Francois BANCHEREAU, San Mateo, CA (US)

(21) Appl. No.: 19/066,376

(22) Filed: Feb. 28, 2025

Related U.S. Application Data
(63) Continuation of application No. PCT/US2023/031612, filed on Aug. 31, 2023.
(60) Provisional application No. 63/374,360, filed on Sep. 1, 2022.

(51) Int. Cl. C07K 16/28; A61K 39/00; A61P 13/10; A61P 35/00; G01N 33/574

(52) U.S. Cl. CPC C07K 16/2818; A61P 13/10; A61P 35/00; C07K 16/2815; C07K 16/2827; G01N 33/57407; A61K 2039/505; G01N 2333/70596; G01N 2800/56

(57) ABSTRACT

The invention provides methods and compositions for treating bladder cancer (e.g., urothelial carcinoma (UC), including locally advanced or metastatic UC) in a subject, for example, by administering a treatment regimen that includes a PD-1 axis binding antagonist (e.g., atezolizumab) to the patient. Also provided are compositions (e.g., a PD-1 axis binding antagonist (e.g., atezolizumab) and/or a platinum-based chemotherapy (e.g., cisplatin or carboplatin and gemcitabine), pharmaceutical compositions thereof, kits thereof, and articles of manufacture thereof) for use in treating bladder cancer (e.g., UC, including locally advanced or metastatic UC) in a patient. Also provided are assays and methods for determining the presence and/or expression level of PD-L1 in a sample obtained from a patient and for labeling PD-L1 in a sample obtained from a patient.

### SEQUENCE LISTING

The instant application contains a Sequence Listing which has been submitted electronically in XML format and is hereby incorporated by reference in its entirety. Said XML copy, created on Feb. 21, 2025, is named 50474-306003_Sequence_Listing_2_21_25.xml and is 12,341 bytes in size.

### FIELD OF THE INVENTION

This invention relates to methods and compositions for use in treating bladder cancer (e.g., urothelial carcinoma (UC), including locally advanced or metastatic UC) in a subject, for example, by administering to the subject a treatment regimen that includes a PD-1 axis binding antagonist (e.g., atezolizumab). The invention also relates to assays and methods for labeling PD-L1 in a tumor sample obtained from a subject.

### BACKGROUND OF THE INVENTION

Cancer remains one of the deadliest threats to human health. Cancers, or malignant tumors, metastasize and grow rapidly in an uncontrolled manner, making timely detection and treatment extremely difficult. Urothelial carcinoma (UC, also termed transitional cell carcinoma (TCC), urothelial bladder cancer or urothelial cell carcinoma (UCC) of the urinary tract) is the most common cancer of the urinary system worldwide with urothelial carcinoma of the bladder being the predominant histologic type and location. Although less common, urothelial carcinoma may also originate in the renal pelvis, ureter, or urethra. It was estimated that in 2015, there would be 74,000 new cases and 16,000 deaths from bladder cancer in the United States. Similar worldwide data estimate that there were 123,000 deaths from bladder cancer in men and 42,000 in women in 2012. The overall 5-year survival rate for metastatic urothelial carcinoma is approximately 5.4%. Poor prognostic factors for survival in patients with metastatic urothelial carcinoma include advanced stage of disease at the time of initial diagnosis, Karnofsky Performance Status <80%, and visceral metastasis. The presence of these unfavorable features was associated with a median survival of 4 months compared with 18 months in patients without these features.

Programmed death-ligand 1 (PD-L1) is a protein that has been implicated in the suppression of immune system responses during cancer, chronic infections, pregnancy, tissue allografts, and autoimmune diseases. PD-L1 regulates the immune response by binding to an inhibitory receptor, known as programmed death 1 (PD-1), which is expressed on the surface of T-cells, B-cells, and monocytes. PD-L1 negatively regulates T-cell function also through interaction with another receptor, B7-1. Formation of the PD-L1/PD-1 and PD-L1/B7-1 complexes negatively regulates T-cell receptor signaling, resulting in the subsequent downregulation of T-cell activation and suppression of anti-tumor immune activity.

Despite the significant advancement in the treatment of cancer (e.g., bladder cancer (e.g., UC, including locally advanced or metastatic UC)), improved therapies are still being sought.

### SUMMARY OF THE INVENTION

This invention relates to, inter alia, methods of treating a bladder cancer (e.g., UC, including locally advanced or metastatic UC) in a subject and compositions (e.g., a PD-1 axis binding antagonist), or a pharmaceutical composition thereof) for use in treating a bladder cancer (e.g., UC, including locally advanced or metastatic UC) in a subject. Also provided are related kits and articles of manufacture. Further provided are assays for determining the presence or expression level of PD-L1 in a tumor sample obtained from a subject suffering from a cancer, methods of labeling PD-L1 in a tumor sample, and methods of stratifying a tumor.

In one aspect, provided herein is a method of treating a locally advanced or metastatic urothelial carcinoma (UC) in a patient in need thereof, wherein the patient is previously untreated for the locally advanced or metastatic UC, the method comprising administering to the patient a treatment regimen comprising an anti-PD-L1 antibody comprising the following hypervariable regions (HVRs): (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8), wherein a tumor sample obtained from the patient has been determined to have: a detectable expression level of PD-L1 in tumor-infiltrating immune cells that comprise 5% or more of the tumor sample using a PD-L1 immunohistochemical (IHC) assay comprising the VENTANA SP142 anti-PD-L1 diagnostic antibody; and a Combined Positive Score (CPS) of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from the treatment regimen comprising the anti-PD-L1 antibody.

In another aspect, provided herein is a method of treating a locally advanced or metastatic UC in a patient in need thereof, wherein the patient is previously untreated for the locally advanced or metastatic UC, the method comprising: (a) determining that a tumor sample obtained from the patient has: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising an anti-PD-L1 antibody comprising the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8); and (b) administering the treatment regimen comprising the anti-PD-L1 antibody to the patient identified in step (a) as one who may benefit from the treatment regimen comprising the anti-PD-L1 antibody.

In another aspect, provided herein is an anti-PD-L1 antibody for use in treatment of a locally advanced or metastatic UC in a patient in need thereof, wherein the patient is previously untreated for the locally advanced or metastatic UC, the treatment comprising administration to the patient of a treatment regimen comprising an anti-PD-L1 antibody comprising the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8), wherein a tumor sample obtained from the patient has been determined to have: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from the treatment regimen comprising the anti-PD-L1 antibody.

In another aspect, provided herein is an anti-PD-L1 antibody for use in a method of treating a locally advanced or metastatic UC in a patient in need thereof, wherein the patient is previously untreated for the locally advanced or metastatic UC, the method comprising: (a) determining that a tumor sample obtained from the patient has: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising an anti-PD-L1 antibody comprising the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8); and (b) administering the treatment regimen comprising the anti-PD-L1 antibody to the patient identified in step (a) as one who may benefit from the treatment regimen comprising the anti-PD-L1 antibody.

In another aspect, provided herein is a method of selecting a therapy for treating a locally advanced or metastatic UC in a patient in need thereof, wherein the patient is previously untreated for the locally advanced or metastatic UC, the method comprising: (a) determining that a tumor sample obtained from the patient has: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising an anti-PD-L1 antibody comprising the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8); and (b) selecting a treatment regimen comprising the anti-PD-L1 antibody for the patient identified in step (a) as one who may benefit from the treatment regimen comprising the anti-PD-L1 antibody.

In another aspect, provided herein is a method of identifying a patient having a locally advanced or metastatic UC who may benefit from a treatment regimen comprising an anti-PD-L1 antibody, wherein the patient is previously untreated for the locally advanced or metastatic UC, the method comprising: determining that a tumor sample obtained from the patient has: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising an anti-PD-L1 antibody, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8)

In some aspects, the method further comprises administering the treatment regimen comprising the anti-PD-L1 antibody to the patient.

In some aspects, the benefit from the treatment regimen comprising the anti-PD-L1 antibody is in terms of overall survival (OS).

In some aspects, the treatment regimen extends the patient's OS by from about 5.7 months to about 17 months as compared to treatment with a platinum-based chemotherapy without the anti-PD-L1 antibody.

In some aspects, the treatment regimen extends the patient's OS by about 11.3 months as compared to treatment with a platinum-based chemotherapy without the anti-PD-L1 antibody.

In some aspects, the platinum-based chemotherapy comprises a platinum-based chemotherapeutic agent and a nucleoside analog.

In some aspects, the platinum-based chemotherapeutic agent is cisplatin, carboplatin, or oxaliplatin.

In some aspects, the platinum-based chemotherapeutic agent is cisplatin.

In some aspects, the platinum-based chemotherapeutic agent is carboplatin.

In some aspects, the nucleoside analog is gemcitabine.

In some aspects, the platinum-based chemotherapy comprises cisplatin and gemcitabine or carboplatin and gemcitabine.

In some aspects, the platinum-based chemotherapy comprises cisplatin and gemcitabine.

In some aspects, the platinum-based chemotherapy comprises carboplatin and gemcitabine.

In some aspects, the anti-PD-L1 antibody comprises: (a) a VH domain comprising the amino acid sequence of SEQ ID NO: 9; and (b) a VL domain comprising the amino acid sequence of SEQ ID NO: 10.

In some aspects, the anti-PD-L1 antibody is atezolizumab.

In some aspects, atezolizumab is administered to the patient intravenously at a dose of about 840 mg every 2 weeks, about 1200 mg every 3 weeks, or about 1680 mg every 4 weeks.

In some aspects, atezolizumab is administered to the patient intravenously at a dose of about 1200 mg every 3 weeks.

In some aspects, atezolizumab is administered to the patient in 21-day dosing cycles, and wherein atezolizumab is administered to the patient intravenously at a dose of about 1200 mg on Day-2 to Day 4 of each 21-day dosing cycle.

In some aspects, atezolizumab is administered to the patient intravenously at a dose of about 1200 mg on Day 1 of each 21-day dosing cycle.

In some aspects, the anti-PD-L1 antibody is administered to the patient as a monotherapy.

In other aspects, the anti-PD-L1 antibody is administered to the patient in combination with one or more additional therapeutic agents.

In some aspects, the one or more additional therapeutic agents comprise a platinum-based chemotherapy.

In some aspects, the platinum-based chemotherapy comprises a platinum-based chemotherapeutic agent and a nucleoside analog.

In some aspects, the platinum-based chemotherapeutic agent is cisplatin, carboplatin, or oxaliplatin.

In some aspects, the platinum-based chemotherapeutic agent is cisplatin.

In some aspects, the platinum-based chemotherapeutic agent is carboplatin.

In some aspects, the nucleoside analog is gemcitabine.

In some aspects, the platinum-based chemotherapy comprises cisplatin and gemcitabine or carboplatin and gemcitabine.

In some aspects, the platinum-based chemotherapy comprises cisplatin and gemcitabine.

In some aspects, the platinum-based chemotherapy comprises carboplatin and gemcitabine.

In some aspects, each dosing cycle for the platinum-based chemotherapy is about 21 days.

In some aspects, cisplatin is administered to the subject intravenously at a dose of about 70 mg/m² on Day-2 to Day 4 of each 21-day dosing cycle.

In some aspects, cisplatin is administered to the subject intravenously at a dose of about 70 mg/m² on Day 1 of each 21-day dosing cycle.

In some aspects, carboplatin is administered to the subject intravenously at an area under the curve (AUC) of about 4.5 on Day-2 to Day 4 of each 21-day dosing cycle.

In some aspects, carboplatin is administered to the subject intravenously at an AUC of about 4.5 on Day 1 of each 21-day dosing cycle.

In some aspects, gemcitabine is administered to the subject intravenously at a dose of about 1000 mg/m² on Day-2 to Day 4 and on Day 7 to Day 11 of each 21-day dosing cycle.

In some aspects, gemcitabine is administered to the subject intravenously at a dose of about 1000 mg/m² on Day 1 and Day 8 of each 21-day dosing cycle.

In some aspects, the patient has not received prior chemotherapy for the locally advanced or metastatic UC.

In some aspects, the patient has previously received an adjuvant or neoadjuvant chemotherapy or chemoradiation for urothelial carcinoma, and has had a treatment-free interval of more than 12 months between the last administration of the adjuvant or neoadjuvant chemotherapy or chemoradiation and the date of recurrence.

In some aspects, the locally advanced or metastatic UC is histologically documented, locally advanced (T4b, any N; or any T, N2-3) or metastatic urothelial carcinoma (mUC) (M1, Stage IV).

In some aspects, the UC is locally advanced UC.

In some aspects, the locally advanced UC is inoperable.

In some aspects, the UC is metastatic UC.

In some aspects, the patient is eligible for treatment with a platinum-based chemotherapy.

In some aspects, the patient is eligible for treatment with a cisplatin-based chemotherapy.

In some aspects, the patient is a human.

In some aspects, the tumor sample obtained from the patient has the presence of discernible PD-L1 staining of any intensity in tumor-infiltrating immune cells covering ≥5% of tumor area occupied by tumor cells, associated intratumoral, and contiguous peritumoral stroma, as determined by the PD-L1 IHC assay comprising the VENTANA SP142 anti-PD-L1 diagnostic antibody.

In some aspects, the tumor sample obtained from the patient has a CPS of ≥10 using a PD-L1 IHC assay comprising the Dako 22C3 anti-PD-L1 diagnostic antibody.

In some aspects, the tumor sample obtained from the patient has a CPS of ≥10 using a PD-L1 IHC assay comprising the VENTANA SP263 anti-PD-L1 diagnostic antibody.

In some aspects, the tumor sample obtained from the patient has a CPS of ≥10 using a PD-L1 IHC assay comprising the 28-8 anti-PD-L1 diagnostic antibody.

In some aspects, the tumor sample is a formalin-fixed and paraffin-embedded (FFPE) tumor sample, an archival tumor sample, a fresh tumor sample, or a frozen tumor sample.

In another aspect, provided herein is an assay for determining the presence or expression level of PD-L1 in a tumor sample obtained from a patient suffering from a cancer, the assay comprising: (a) determining the presence or expression level of PD-L1 in a tumor sample obtained from the patient using a PD-L1 IHC assay comprising the VENTANA SP142 anti-PD-L1 diagnostic antibody; and (b) determining the presence or expression level of PD-L1 in the tumor sample obtained from the patient using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody.

In some aspects, the tumor sample obtained from the patient has a detectable expression level of PD-L1 in tumor-infiltrating immune cells that comprise 5% or more of the tumor sample using a PD-L1 IHC assay comprising the VENTANA SP142 anti-PD-L1 diagnostic antibody.

In some aspects, the tumor sample obtained from the patient has the presence of discernible PD-L1 staining of any intensity in tumor-infiltrating immune cells covering ≥5% of tumor area occupied by tumor cells, associated intratumoral, and contiguous peritumoral stroma, as determined by the PD-L1 IHC assay comprising the VENTANA SP142 anti-PD-L1 diagnostic antibody.

In some aspects, the tumor sample obtained from the patient has a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody.

In some aspects, the tumor sample obtained from the patient has: a detectable expression level of PD-L1 in tumor-infiltrating immune cells that comprise 5% or more of the tumor sample using the PD-L1 IHC assay comprising the VENTANA SP142 anti-PD-L1 diagnostic antibody; and a CPS of ≥10 using the PD-L1 IHC assay comprising the Dako 22C3 anti-PD-L1 diagnostic antibody.

In some aspects, steps (a) and (b) are performed simultaneously.

In some aspects, steps (a) and (b) are performed sequentially.

In some aspects, steps (a) and (b) are performed in different sections of the tumor sample or in the same section of the tumor sample.

In some aspects, the different sections of the tumor sample are consecutive sections.

In some aspects, the cancer is locally advanced or metastatic urothelial carcinoma.

In some aspects, the patient is previously untreated for the locally advanced or metastatic urothelial carcinoma.

In some aspects, the assay is used for (i) selecting a therapy for treating a locally advanced or metastatic UC in a patient in need thereof or (ii) identifying a patient having a locally advanced or metastatic UC who may benefit from a treatment regimen comprising an anti-PD-L1 antibody.

In some aspects, the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some aspects, the tumor sample is an FFPE tumor sample, an archival tumor sample, a fresh tumor sample, or a frozen tumor sample.

In another aspect, provided herein is a method of labeling PD-L1 in a tumor sample, the method comprising the following steps: (a) contacting the tumor sample with the VENTANA SP142 anti-PD-L1 diagnostic antibody; (b) contacting the tumor sample with the Dako 22C3 anti-PD-L1 diagnostic antibody, the VENTANA SP263 anti-PD-L1 diagnostic antibody, or the 28-8 anti-PD-L1 diagnostic antibody; and (c) visualizing the anti-PD-L1 diagnostic antibodies of steps (a) and (b) with one or more detectable reagents that generates a detectable signal for both of the anti-PD-L1 diagnostic antibodies.

In some aspects, the detectable signal for the VENTANA SP142 anti-PD-L1 diagnostic antibody is an amplified signal.

In some aspects, the amplified signal is generated by tyramide signal amplification.

In some aspects, steps (a) and (b) are performed simultaneously.

In some aspects, steps (a) and (b) are performed sequentially.

In some aspects, steps (a) and (b) are performed in different sections of the tumor sample or in the same section of the tumor sample.

In some aspects, the different sections of the tumor sample are consecutive sections.

In some aspects, the visualizing comprises IHC or immunofluorescence (IF).

In some aspects, the visualizing comprises IHC.

In some aspects, the tumor sample is an FFPE tumor sample, an archival tumor sample, a fresh tumor sample, or a frozen tumor sample.

In some aspects, the tumor sample is obtained from a patient having a cancer.

In some aspects, the cancer is locally advanced or metastatic urothelial carcinoma.

In some aspects, the patient is previously untreated for the locally advanced or metastatic urothelial carcinoma.

In another aspect, provided herein is a kit comprising: (a) the VENTANA SP142 anti-PD-L1 diagnostic antibody; and (b) the Dako 22C3 anti-PD-L1 diagnostic antibody, the VENTANA SP263 anti-PD-L1 diagnostic antibody, or the 28-8 anti-PD-L1 diagnostic antibody.

In some aspects, the kit further comprises one or more reagents for visualizing the anti-PD-L1 diagnostic antibodies of (a) and (b).

In another aspect, provided herein is a method of identifying a tumor likely to respond to a PD-1 axis binding antagonist, the method comprising: (a) staining a first portion of the tumor with an immune-directed PD-L1 assay to obtain a first stained sample; (b) generating a first score by applying a first scoring algorithm to the first stained sample; (c) staining a second portion of the tumor with an immune-agnostic PD-L1 assay to obtain a second stained sample; (d) generating a second score by applying a second scoring algorithm the second stained sample; and (e) comparing the first score to a first cutoff and the second score to a second cutoff, wherein the tumor is likely to respond to the PD-1 axis binding antagonist when both the first score meets or exceeds the first cutoff and the second score meets or exceeds the second cutoff.

In some aspects, the immune-directed PD-L1 assay has: (i) at least an 80% overall percent agreement (OPA) with an SP142 Assay using the first scoring algorithm at the first cutoff value; (ii) at least an 80% positive percent agreement (PPA) with an SP142 Assay using the first scoring algorithm at the first cutoff value; (iii) at least an 80% negative percent agreement (NPA) with an SP142 Assay using the first scoring algorithm at the first cutoff value; (iv) at least an 80% PPA and at least an 80% NPA with an SP142 Assay using the first scoring algorithm at the first cutoff value; (v) at least an 80% PPA and at least an 80% OPA with an SP142 Assay using the first scoring algorithm at the first cutoff value; (vi) at least an 80% NPA and at least an 80% OPA with an SP142 Assay using the first scoring algorithm at the first cutoff value; and/or (vii) at least an 80% OPA, at least an 80% PPA, and at least an 80% NPA with an SP142 Assay using the first scoring algorithm at the first cutoff value.

In some aspects, the immune-agnostic PD-L1 assay has: (i) at least an 80% overall percent agreement (OPA) with an 22C3 Assay using the second scoring algorithm at the second cutoff value; (ii) at least an 80% positive percent agreement (PPA) with an 22C3 Assay using the second scoring algorithm at the second cutoff value; (iii) at least an 80% negative percent agreement (NPA) with an 22C3 Assay using the second scoring algorithm at the second cutoff value; (iv) at least an 80% PPA and at least an 80% NPA with an 22C3 Assay using the second scoring algorithm at the second cutoff value; (v) at least an 80% PPA and at least an 80% OPA with an 22C3 Assay using the second scoring algorithm at the second cutoff value; (vi) at least an 80% NPA and at least an 80% OPA with an 22C3 Assay using the second scoring algorithm at the second cutoff value; and/or (vii) at least an 80% OPA, at least an 80% PPA, and at least an 80% NPA with an 22C3 Assay using the second scoring algorithm at the second cutoff value.

In another aspect, provided herein is a method of stratifying a tumor having a score with an immune-agnostic PD-L1 assay that exceeds a pre-determined cutoff, the method comprising: (a) staining a portion of the tumor with an immune-directed PD-L1 assay to obtain a stained sample; (b) generating a score by applying a scoring algorithm to the stained sample; and (c) comparing the score to a first cutoff, wherein the tumor is likely to respond to a PD-1 axis binding antagonist when both the first score meets or exceeds the first cutoff.

In another aspect, provided herein is a method of stratifying a CPS ≥10% tumor as determined by a 22C3 assay, the method comprising: (a) staining a portion of the tumor with an immune-directed PD-L1 assay to obtain a stained sample; (b) generating a score by applying a scoring algorithm to the stained sample; and (c) comparing the score to a first cutoff and the second score to a second cutoff, wherein the tumor is likely to respond to the PD-1 axis binding antagonist when both the first score meets or exceeds the first cutoff and the second score meets or exceeds the second cutoff.

In another aspect, provided herein is a method of stratifying a CPS ≥10% tumor as determined by an SP263 assay, the method comprising: (a) staining a portion of the tumor with an immune-directed PD-L1 assay to obtain a stained sample; (b) generating a score by applying a scoring algorithm to the stained sample; and (c) comparing the score to a first cutoff and the second score to a second cutoff, wherein the tumor is likely to respond to the PD-1 axis binding antagonist when both the first score meets or exceeds the first cutoff and the second score meets or exceeds the second cutoff.

In another aspect, provided herein is a method of stratifying a CPS ≥10% tumor as determined by a 28-8 assay, the method comprising: (a) staining a portion of the tumor with an immune-directed PD-L1 assay to obtain a stained sample; (b) generating a score by applying a scoring algorithm to the stained sample; and (c) comparing the score to a first cutoff and the second score to a second cutoff, wherein the tumor is likely to respond to the PD-1 axis binding antagonist when both the first score meets or exceeds the first cutoff and the second score meets or exceeds the second cutoff.

In some aspects, the tumor is a locally advanced or metastatic UC.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 is a schematic diagram for the Phase III IMvigor130 study (NCT02807636). mUC, metastatic urothelial carcinoma; R, randomized; ECOG PS, Eastern Cooperative Oncology Group performance status; 1L, first line; platinum, investigator's choice of cisplatin or carboplatin.

FIG. 2 is a schematic diagram showing the change in the study design in the IMvigor130 study from a 2-arm to a 3-arm design. The bottom panel shows a table summarizing design changes. Atezo, atezolizumab; carbo, carboplatin; cis, cisplatin; mono, monotherapy; PFS, progression-free survival; OS, overall survival.

FIG. 3 is a schematic diagram showing evolution of study design for IMvigor130. FPI: first patient in; LPI: last patient in.

FIG. 4 is a schematic diagram depicting the hierarchical relationship between study endpoints for type I error control.

FIGS. 5A-5C are a series of images showing the results of immunohistochemistry (IHC) in urothelial carcinoma (UC) tumor tissue using the anti-PD-L1 antibody SP142 (FIG. 5A), anti-DC-LAMP antibody (FIG. 5B), and a combination of the anti-PD-L1 antibody SP142 and the anti-DC-LAMP antibody (FIG. 5C). Double labeling from SP142 (brown) and the anti-DC-LAMP antibody (green) is shown (FIG. 5C).

FIGS. 6A-6C are a series of graphs showing bulk RNA sequencing deconvolution data for the percent inferred population frequency of dendritic cells (FIG. 6A), CD8+ T cells (FIG. 6B), and CD4+ T cells (FIG. 6C) in urothelial carcinoma tissue samples identified as double-negative for 22C3 and SP142 (DN), 22C3 positive only (22C3), SP142 positive (SP142), and double-positive for 22C3 and SP142 (DP).

FIGS. 7A and 7B are a series of graphs showing Kaplan-Meier plots of overall survival (OS) for patients receiving atezolizumab monotherapy comparing PD-L1 IHC assay tumor tissue scores by the SP142 assay (FIG. 7A) and 22C3 assay (FIG. 7B). OS for patients with PD-L1-stained tumor-infiltrating immune cells (IC) scoring of IC0/1 (red) versus IC2/3 (blue) is shown for the SP142 IHC assay (FIG. 7A). OS for patients with combined positive score (CPS)<10 (red) versus CPS ≥10 (blue) is shown for the 22C3 assay (FIG. 7B). CPS, combined positive score; CI, confidence interval; HR, hazard ratio; OS, overall survival; NE, not estimable.

FIGS. 8A-8D are a series of graphs showing Kaplan-Meier plots of OS for patients receiving atezolizumab monotherapy (Arm B; atezo) versus placebo plus platinum plus gemcitabine chemotherapy (Arm C; chemo) by PD-L1 IHC assay tumor tissue scores of the SP142 assay (FIGS. 8A and 8C) and 22C3 assay (FIGS. 8B and 8D). OS for patients with IC scoring of IC0/1 receiving atezolizumab monotherapy (red) versus chemotherapy (blue) is shown for the SP142 IHC assay (FIG. 8A), and OS for patients with CPS <10 receiving atezolizumab monotherapy (red) versus chemotherapy (blue) is shown for the 22C3 assay (FIG. 8B). OS for patients with IC scoring of IC2/3 receiving atezolizumab monotherapy (green) versus chemotherapy (purple) is shown for the SP142 IHC assay (FIG. 8C), and OS for patients with CPS ≥10 receiving atezolizumab monotherapy (green) versus chemotherapy (purple) is shown for the 22C3 assay (FIG. 8D).

FIGS. 9A-9D are a series of graphs showing Kaplan-Meier plots of OS for patients receiving atezolizumab monotherapy (Arm B; atezo) versus patients receiving chemotherapy who are cisplatin-ineligible (i.e., placebo plus carboplatin plus gemcitabine) (Arm C; chemo). The study arms are shown by the PD-L1 IHC assay tumor tissue scores of the SP142 assay (FIGS. 9A and 9C) and 22C3 assay (FIGS. 9B and 9D). OS for patients with IC scoring of IC0/1 receiving atezolizumab monotherapy (red) versus chemotherapy (blue) is shown for the SP142 IHC assay (FIG. 9A), and OS for patients with CPS<10 receiving atezolizumab monotherapy (red) versus chemotherapy (blue) is shown for the 22C3 assay (FIG. 9B). OS for patients with IC scoring of IC2/3 receiving atezolizumab monotherapy (green) versus chemotherapy (purple) is shown for the SP142 IHC assay (FIG. 9C), and OS for patients with CPS≥10 receiving atezolizumab monotherapy (green) versus chemotherapy (purple) is shown for the 22C3 assay (FIG. 9D).

FIGS. 10A and 10B are a series of graphs showing Kaplan-Meier plots of OS for patients receiving atezolizumab monotherapy (Arm B; atezo) (FIG. 10A) and patients receiving placebo plus platinum plus gemcitabine (Arm C; chemo) (FIG. 10B) who are further shown by double-positive staining from the SP142 and 22C3 assays. Within each treatment arm, the OS for patients with SP142 IC0/1 and 22C3 CPS<10 (red), SP142 IC0/1 and 22C3 CPS≥10 (green), SP142 IC2/3 and 22C3 CPS<10 (blue), and SP142 IC2/3 and 22C3 CPS≥10 (purple) is shown for patients receiving atezolizumab monotherapy (FIG. 10A) and patients receiving placebo plus carboplatin plus gemcitabine (FIG. 10B).

FIG. 11 is a schematic diagram showing an exemplary method using the OptiView DAB IHC Detection Kit. DAB, 3,3′-diaminobenzidine tetrahydrochloride; HQ, proprietary hapten attached to the goat antibodies.

### DETAILED DESCRIPTION OF THE INVENTION

The present invention provides therapeutic methods and compositions for cancer, for example, bladder cancer (e.g., UC, including locally advanced or metastatic UC), including in patients who have not been previously treated for their cancer. The invention is based, at least in part, on the discovery that PD-L1 expression on immune cells as assessed by the SP142 antibody co-localizes with dendritic cells and is associated with improved overall survival (OS) from treatment with the anti-PD-L1 antibody atezolizumab in patients with untreated locally advanced or metastatic UC. Unexpectedly, longer OS was associated with SP142 IC2/3+22C3 CPS ≥10 tumor status, while shorter OS was observed in patients with tumors staining for SP142 IC0/1+22C3 CPS ≥10. Therefore, analyzing tumor samples from cancer patients using assays and methods in which PD-L1 is detected with both an immune-directed PD-L1 AHC assay (e.g., an SP142 IHC assay) and an immune-agnostic PD-L1 AHC assay (e.g., an 22C3, SP263, or 28-8 IHC assay) can be useful, e.g., for identifying patients who are likely to benefit from immune checkpoint inhibitors such as PD-1 axis binding antagonists (e.g., atezolizumab).

### I. Definitions

The following abbreviations are used herein:

| | |
|---|---|
| AHC | affinity histochemical |
| ACC | affinity cytochemical |
| CAS | Chemical Abstracts Service |
| CDR | complementarity determining region |
| CR | complete response |
| DNA | deoxyribonucleic acid |
| DOR | duration of response |
| Fab | fragment antigen-binding |
| Fc | fragment crystallizable |
| FFPE | formalin-fixed and paraffin-embedded |
| FR | framework |
| HVR | hypervariable region |
| IHC | immunohistochemistry or |
| | immunohistochemical |
| ORR | overall response rate/objective |
| | response rate |
| OS | overall survival |
| PD-1 | programmed death 1 |
| PD-L1 | programmed death ligand 1 |
| PD-L2 | programmed death ligand 2 |
| PFS | progression-free survival |
| PR | partial response |
| RNA | ribonucleic acid |
| SLD | sum of the longest diameters |

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L2 binding antagonist. In some instances, the PD-1 axis binding antagonist includes a PD-L1 binding antagonist or a PD-1 binding antagonist. In a preferred aspect, the PD-1 axis binding antagonist is a PD-L1 binding antagonist.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some aspects, the anti-PD-L1 antibody is atezolizumab, MDX-1105, MEDI4736 (durvalumab), or MSB0010718C (avelumab). In one specific aspect, the PD-L1 binding antagonist is MDX-1105. In another specific aspect, the PD-L1 binding antagonist is MEDI4736 (durvalumab). In another specific aspect, the PD-L1 binding antagonist is MSB0010718C (avelumab). In other aspects, the PD-L1 binding antagonist may be a small molecule, e.g., GS-4224, INCB086550, MAX-10181, INCB090244, CA-170, or ABSK041, which in some instances may be administered orally. Other exemplary PD-L1 binding antagonists include AVA-004, MT-6035, VXM10, LYN192, GB7003, and JS-003. In a preferred aspect, the PD-L1 binding antagonist is atezolizumab.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. In some instances, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one instance, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some instances, the PD-1 binding antagonist binds to PD-1. In some instances, the PD-1 binding antagonist is an anti-PD-1 antibody (e.g., an anti-PD-1 antagonist antibody). Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is a PD-L2 Fc fusion protein, e.g., AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In another specific aspect, a PD-1 binding antagonist is prolgolimab. In another specific aspect, a PD-1 binding antagonist is camrelizumab. In another specific aspect, a PD-1 binding antagonist is sintilimab. In another specific aspect, a PD-1 binding antagonist is tislelizumab. In another specific aspect, a PD-1 binding antagonist is toripalimab. Other additional exemplary PD-1 binding antagonists include BION-004, CB201, AUNP-012, ADG104, and LBL-006.

The term “PD-L2 binding antagonist” refers to a molecule that decreases, blocks, inhibits, abrogates or interferes with signal transduction resulting from the interaction of PD-L2 with either one or more of its binding partners, such as PD-1. PD-L2 (programmed death ligand 2) is also referred to in the art as “programmed cell death 1 ligand 2,” “PDCD1LG2,” “CD273,” “B7-DC,” “Btdc,” and “PDL2.” An exemplary human PD-L2 is shown in UniProtKB/Swiss-Prot Accession No. Q9BQ51. In some instances, a PD-L2 binding antagonist is a molecule that inhibits the binding of PD-L2 to one or more of its binding partners. In a specific aspect, the PD-L2 binding antagonist inhibits binding of PD-L2 to PD-1. Exemplary PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some aspects, the PD-L2 binding antagonist binds to PD-L2. In some aspects, a PD-L2 binding antagonist is an immunoadhesin. In other aspects, a PD-L2 binding antagonist is an anti-PD-L2 antagonist antibody.

The terms “programmed death ligand 1” and “PD-L1” refer herein to native sequence human PD-L1 polypeptide. Native sequence PD-L1 polypeptides are provided under Uniprot Accession No. Q9NZQ7. For example, the native sequence PD-L1 may have the amino acid sequence as set forth in Uniprot Accession No. Q9NZQ7-1 (isoform 1). In another example, the native sequence PD-L1 may have the amino acid sequence as set forth in Uniprot Accession No. Q9NZQ7-2 (isoform 2). In yet another example, the native sequence PD-L1 may have the amino acid sequence as set forth in Uniprot Accession No. Q9NZQ7-3 (isoform 3). PD-L1 is also referred to in the art as “programmed cell death 1 ligand 1,” “PDCD1LG1,” “CD274,” “B7-H,” and “PDL1.”

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

For the purposes herein, “atezolizumab” is an Fc-engineered, humanized, non-glycosylated IgG1 kappa immunoglobulin that binds PD-L1 and comprises the heavy chain sequence of SEQ ID NO: 1 and the light chain sequence of SEQ ID NO: 2. Atezolizumab comprises a single amino acid substitution (asparagine to alanine) at position 297 on the heavy chain (N297A) using EU numbering of Fc region amino acid residues, which results in a non-glycosylated antibody that has minimal binding to Fc receptors. Atezolizumab is also described in WHO Drug Information (International Nonproprietary Names for Pharmaceutical Substances (proposed INN)) List 112, Vol. 28, No. 4, 2014, p. 488.

The term “cancer” refers to a disease caused by an uncontrolled division of abnormal cells in a part of the body. Aspects of cancer include solid tumor cancers and non-solid tumor cancers. Examples of cancer include but are not limited to, carcinoma, lymphoma, blastoma, sarcoma, and leukemia or lymphoid malignancies. More particular examples of such cancers include, but are not limited to, bladder cancer (e.g., urothelial carcinoma (UC), including metastatic UC (mUC); muscle-invasive bladder cancer (MIBC), and non-muscle-invasive bladder cancer (NMIBC)); kidney or renal cancer (e.g., renal cell carcinoma (RCC)); lung cancer, including small-cell lung cancer, non-small cell lung cancer, adenocarcinoma of the lung, and squamous carcinoma of the lung; cancer of the urinary tract; breast cancer (e.g., HER2+ breast cancer and triple-negative breast cancer (TNBC), which are estrogen receptors (ER−), progesterone receptors (PR−), and HER2 (HER2−) negative); prostate cancer, such as castration-resistant prostate cancer (CRPC); cancer of the peritoneum; hepatocellular cancer; gastric or stomach cancer, including gastrointestinal cancer and gastrointestinal stromal cancer; pancreatic cancer (e.g., pancreatic ductal adenocarcinoma (PDAC)); glioblastoma; cervical cancer; ovarian cancer; liver cancer (e.g., hepatocellular carcinoma (HCC)); hepatoma; colon cancer; rectal cancer; colorectal cancer; endometrial or uterine carcinoma; salivary gland carcinoma; prostate cancer; vulval cancer; thyroid cancer; hepatic carcinoma; anal carcinoma; penile carcinoma; melanoma, including superficial spreading melanoma, lentigo malignant melanoma, acral lentiginous melanomas, and nodular melanomas; multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases. In one instance, the cancer is bladder cancer, e.g., urothelial carcinoma (UC) (e.g., locally advanced or metastatic UC). The cancer may be locally advanced or metastatic. In some instances, the cancer is locally advanced. In other instances, the cancer is metastatic. In some instances, the cancer may be unresectable (e.g., unresectable locally advanced or metastatic cancer). In some embodiments, the UC is locally advanced UC. In some embodiments, the locally advanced UC is inoperable. In some embodiments, the UC is metastatic UC (mUC). In some embodiments, the locally advanced or metastatic UC is histologically documented, locally advanced (T4b, any N; or any T, N2-3) or metastatic urothelial carcinoma (mUC) (M1, Stage IV).

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder,” and “tumor” are not mutually exclusive as referred to herein.

The terms “cell proliferative disorder” and “proliferative disorder” refer to disorders that are associated with some degree of abnormal cell proliferation. In one embodiment, the cell proliferative disorder is cancer. In another embodiment, the cell proliferative disorder is a tumor.

The term “bladder cancer” includes, but is not limited to, urothelial carcinoma (UC), and which may be, for example, locally advanced or metastatic. The methods described herein are suitable for treatment of various stages of cancer, including cancers that are locally advanced and/or metastatic. In cancer staging, locally advanced is generally defined as cancer that has spread from a localized area to nearby tissues and/or lymph nodes. In the Roman numeral staging system, locally advanced usually is classified in Stage II or III. Cancer which is metastatic is a stage where the cancer spreads throughout the body to distant tissues and organs (stage IV).

As used herein, “treating” comprises effective cancer treatment with an effective amount of a therapeutic agent (e.g., a PD-1 axis binding antagonist (e.g., atezolizumab) or combination of therapeutic agents (e.g., a PD-1 axis antagonist and one or more additional therapeutic agents, e.g., a platinum-based chemotherapy (e.g., gemcitabine with either cisplatin or carboplatin)). Treating herein includes, inter alia, adjuvant therapy, neoadjuvant therapy, non-metastatic cancer therapy (e.g., locally advanced cancer therapy), and metastatic cancer therapy. The treatment may be first-line treatment (e.g., the patient may be previously untreated or not have received prior systemic therapy), or second line or later treatment. In particular examples, the treatment may be first-line treatment (e.g., the patient may be previously untreated for the locally advanced or metastatic urothelial carcinoma).

Herein, an “effective amount” refers to the amount of a therapeutic agent (e.g., a PD-1 axis binding antagonist (e.g., atezolizumab) or a combination of therapeutic agents (e.g., a PD-1 axis antagonist and one or more additional therapeutic agents, e.g., a platinum-based chemotherapy (e.g., gemcitabine with either cisplatin or carboplatin))), that achieves a therapeutic result. In some examples, the effective amount of a therapeutic agent or a combination of therapeutic agents is the amount of the agent or of the combination of agents that achieves a clinical endpoint of improved overall response rate (ORR), a complete response (CR), a partial response (PR), a disease control rate (DCR), improved survival (e.g., progression-free survival (PFS) and/or overall survival (OS)), and/or improved duration of response (DOR). Improvement (e.g., in terms of response rate (e.g., ORR, CR, PR, and/or DCR), survival (e.g., PFS and/or OS), or DOR) may be relative to a suitable reference treatment, for example, treatment that does not include the PD-1 axis binding antagonist. For example, treatment with an anti-cancer therapy that includes an anti-PD-L1 antibody (e.g., atezolizumab) may be compared with a reference treatment, which may be a treatment with a platinum-based chemotherapy without the anti-PD-L1 antibody.

As used herein, “complete response” and “CR” refers to disappearance of all target lesions.

As used herein, “partial response” and “PR” refers to at least a 30% decrease in the sum of the longest diameters (SLD) of target lesions, taking as reference the baseline SLD prior to treatment.

As used herein, “disease control rate” and “DCR” refers to the proportion of patients with confirmed CR or PR as best response, or stable disease (SD). For example, in some embodiments, DCR may be defined as the proportion of patients with confirmed CR or PR as best response, or stable disease maintained for ≥6 months, per RECIST v1.1.

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

As used herein, “progression-free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease. In some embodiments, PFS may be defined as the time from randomization or the beginning of treatment to the first documented disease progression as assessed by RECIST v1.1, or death from any cause, whichever occurs first.

As used herein, “objective response rate” or “objective response rate” (ORR) refers to the sum of complete response (CR) rate and partial response (PR) rate. For example, in some embodiments, ORR refers to the proportion of patients with a confirmed objective response, either CR or PR, observed on two assessments greater than or equal to 28 days apart per RECIST v1.1, based on investigator assessment.

As used herein, “overall survival” and “OS” refer to the length of time from either the date of diagnosis or the start of treatment for a disease (e.g., cancer) that the patient is still alive. In some embodiments, OS is defined as the time from randomization to death due to any cause.

As used herein, the term “duration of response” (DOR) refers to a length of time from documentation of a tumor response until disease progression or death from any cause, whichever occurs first.

As used herein, the terms “inoperable” and “unresectable” are used interchangeably to refer to a cancer (e.g., bladder cancer (e.g., UC, including locally advanced or metastatic UC)) for which surgical resection is not possible or cannot be safely performed. In some embodiments, a bladder cancer (e.g., UC, including locally advanced or metastatic UC) is inoperable on the basis of involvement of pelvic sidewall or adjacent viscera (clinical Stage T4b) or bulky nodal metastasis (N2-N3).

The term “eligible for treatment with a platinum-based chemotherapy” means that the subject is eligible for treatment with a platinum-based chemotherapy, either in the attending clinician's judgment or according to standardized criteria for eligibility for platinum-based chemotherapy that are known in the art. For example, the criteria set forth in Galsky et al. Lancet Oncol. 12 (3): 211-4, 2011 may be used to determine whether a subject is eligible for cisplatin-based chemotherapy. Galsky et al. describe a consensus definition of patients with metastatic UC (mUC) in which patients meeting at least one of the following are considered unfit for cisplatin-based chemotherapy: (i) a World Health Association (WHO) or Eastern Cooperative Oncology Group (ECOG) performance status of 2, or Karnofsky performance status of 60-70%; (ii) creatinine clearance (calculated or measured) less than 1 mL/s; (iii) National Cancer Institute (NCI) Common Terminology Criteria for Adverse Events (CTCAE) v4.0 Grade ≥2 audiometric hearing loss; (iv) CTCAE v.4.0 Grade ≥2 peripheral neuropathy; and/or New York Heart Association (NYHA) class III heart failure. In one example, a patient is considered unfit for cisplatin-based chemotherapy if they have one or more of the following: impaired renal function (e.g., glomerular filtration rate (GFR)>30 but <60 mL/min); GFR may be assessed by direct measurement (i.e., creatinine clearance or ethyldediaminetetra-acetate) or, if not available, by calculation from serum/plasma creatinine (Cockcroft-Gault formula)); hearing loss (e.g., National Cancer Institute (NCI) Common Terminology Criteria for Adverse Events (CTCAE) v4.0 Grade ≥2 audiometric hearing loss of 25 decibels at two contiguous frequencies); peripheral neuropathy (e.g., NCI CTCAE v4.0 Grade ≥2 peripheral neuropathy (i.e., sensory alteration or paresthesia, including tingling)); and/or ECOG performance status assessment (see Oken et al. Am. J. Clin. Oncol. 5:649-655, 1982) (e.g., an ECOG performance status of 2). In some embodiments, a subject having one of the following may be eligible for carboplatin-based chemotherapy: impaired renal function (e.g., glomerular filtration rate (GFR)>30 but <60 mL/min); GFR may be assessed by direct measurement (i.e., creatinine clearance or ethyldediaminetetra-acetate) or, if not available, by calculation from serum/plasma creatinine (Cockcroft-Gault formula)); hearing loss (e.g., CTCAE v4.0 Grade ≥2 audiometric hearing loss of 25 decibels at two contiguous frequencies); peripheral neuropathy (e.g., NCI CTCAE v4.0 Grade ≥2 peripheral neuropathy (i.e., sensory alteration or paresthesia, including tingling)); and/or ECOG performance status assessment (e.g., an ECOG performance status of 2).

As used herein, the term “chemotherapeutic agent” refers to a compound useful in the treatment of cancer, such as bladder cancer, e.g., UC (e.g., locally advanced or metastatic UC). Examples of chemotherapeutic agents include EGFR inhibitors (including small molecule inhibitors (e.g., erlotinib (TARCEVA®, Genentech/OSI Pharm.); PD 183805 (CI 1033, 2-propenamide, N-[4-[(3-chloro-4-fluorophenyl)amino]-7-[3-(4-morpholinyl) propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy) quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl) methoxy]phenyl]-6[5[[[2methylsulfonyl)ethyl]amino]methyl]-2-furanyl]-4-quinazolinamine)); a tyrosine kinase inhibitor (e.g., an EGFR inhibitor; a small molecule HER2 tyrosine kinase inhibitor such as TAK165 (Takeda); CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; PKI-166 (Novartis); pan-HER inhibitors such as canertinib (CI-1033; Pharmacia); Raf-1 inhibitors such as antisense agent ISIS-5132 (ISIS Pharmaceuticals) which inhibit Raf-1 signaling; non-HER-targeted tyrosine kinase inhibitors such as imatinib mesylate (GLEEVEC®, Glaxo SmithKline); multi-targeted tyrosine kinase inhibitors such as sunitinib (SUTENT®, Pfizer); VEGF receptor tyrosine kinase inhibitors such as vatalanib (PTK787/ZK222584, Novartis/Schering AG); MAPK extracellular regulated kinase I inhibitor CI-1040 (Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis(4-fluoroanilino) phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone); and rapamycin (sirolimus, RAPAMUNE®)); proteasome inhibitors such as bortezomib (VELCADE®, Millennium Pharm.); disulfiram; epigallocatechin gallate; salinosporamide A; carfilzomib; 17-AAG (geldanamycin); radicicol; lactate dehydrogenase A (LDH-A); fulvestrant (FASLODEX®, AstraZeneca); letrozole (FEMARA®, Novartis), finasunate (VATALANIB®, Novartis); oxaliplatin (ELOXATIN®, Sanofi); 5-FU (5-fluorouracil); leucovorin; lonafamib (SCH 66336); sorafenib (NEXAVAR®, Bayer Labs); AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin γ1 and calicheamicin ω1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, cactinomycin, carabicin, caminomycin, carzinophilin, chromomycinis, dactinomycin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; chloranmbucil; GEMZAR® (gemcitabine); 6-thioguanine; mercaptopurine; methotrexate; etoposide (VP-16); ifosfamide; mitoxantrone; novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids, prodrugs, and derivatives of any of the above.

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4 (5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTING, LEUVECTIN®, and VAXID®; (ix) growth inhibitory agents including vincas (e.g., vincristine and vinblastine), NAVELBINE® (vinorelbine), taxanes (e.g., paclitaxel, nab-paclitaxel, and docetaxel), topoisomerase II inhibitors (e.g., doxorubicin, epirubicin, daunorubicin, etoposide, and bleomycin), and DNA alkylating agents (e.g., tamoxigen, prednisone, dacarbazine, mechlorethamine, cisplatin, methotrexate, 5-fluorouracil, and ara-C); and (x) pharmaceutically acceptable salts, acids, prodrugs, and derivatives of any of the above.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one instance, the cytotoxic agent is a platinum-based chemotherapeutic agent (e.g., carboplatin or cisplatin). In one instance, the cytotoxic agent is an antagonist of EGFR, e.g., N-(3-ethynylphenyl)-6,7-bis(2-methoxyethoxy) quinazolin-4-amine (e.g., erlotinib). In one instance the cytotoxic agent is a RAF inhibitor, e.g., a BRAF and/or CRAF inhibitor. In one instance the RAF inhibitor is vemurafenib. In one instance, the cytotoxic agent is a PI3K inhibitor.

Chemotherapeutic agents also include “platinum-based” chemotherapeutic agents, which comprise an organic compound which contains platinum as an integral part of the molecule. Typically, platinum-based chemotherapeutic agents are coordination complexes of platinum. Platinum-based chemotherapeutic agents are sometimes called “platins” in the art. Examples of platinum-based chemotherapeutic agents include, but are not limited to, cisplatin, carboplatin, oxaliplatin, nedaplatin, triplatin tetranitrate, phenanthriplatin, picoplatin, lipoplatin, and satraplatin. Platinum-based chemotherapeutic agents (e.g., cisplatin or carboplatin) may be administered in combination with one or more additional chemotherapeutic agents, e.g., a nucleoside analog (e.g., gemcitabine).

A “platinum-based chemotherapy,” as used herein, refers to a chemotherapy regimen that includes a platinum-based chemotherapeutic agent. For example, a platinum-based chemotherapy may include a platinum-based chemotherapeutic agent (e.g., cisplatin or carboplatin) in combination with one or more additional chemotherapeutic agents, e.g., a nucleoside analog (e.g., gemcitabine).

A “nucleoside analog,” as used herein, refers to a nucleoside that includes a nucleic acid analog and a sugar. Nucleoside analogs may function as antimetabolites. Exemplary nucleoside analogues include but are not limited to gemcitabine, cytarabine, fludarabine, and cladribine.

The term “patient” refers to a human patient. For example, the patient may be an adult.

The term “antibody” herein specifically covers monoclonal antibodies (including full-length monoclonal antibodies), polyclonal antibodies, multispecific antibodies (e.g., bispecific antibodies), and antibody fragments so long as they exhibit the desired biological activity. In one instance, the antibody is a full-length monoclonal antibody.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms refer to an antibody comprising an Fc region.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447). Therefore, the C-terminal lysine (Lys447), or the C-terminal glycine (Gly446) and lysine (Lys447), of the Fc region may or may not be present. Amino acid sequences of heavy chains including an Fc region are denoted herein without the

C-terminal lysine (Lys447) if not indicated otherwise. In one aspect, a heavy chain including an Fc region as specified herein, comprised in an antibody disclosed herein, comprises an additional C-terminal glycine-lysine dipeptide (G446 and K447). In one aspect, a heavy chain including an Fc region as specified herein, comprised in an antibody disclosed herein, comprises an additional C-terminal glycine residue (G446). In one aspect, a heavy chain including an Fc region as specified herein, comprised in an antibody disclosed herein, comprises an additional C-terminal lysine residue (K447). In one embodiment, the Fc region contains a single amino acid substitution N297A of the heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

A “naked antibody” refers to an antibody that is not conjugated to a heterologous moiety (e.g., a cytotoxic moiety) or radiolabel. The naked antibody may be present in a pharmaceutical composition.

“Antibody fragments” comprise a portion of an intact antibody, preferably comprising the antigen-binding region thereof. In some instances, the antibody fragment described herein is an antigen-binding fragment. Examples of antibody fragments include Fab, Fab′, F(ab′)₂, and Fv fragments; diabodies; linear antibodies; single-chain antibody molecules (e.g., scFvs); and multispecific antibodies formed from antibody fragments.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1 (CDR-L1)-FR2-CDR-H2 (CDR-L2)-FR3-CDR-H3 (CDR-L3)-FR4.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “in combination with” refers to administration of one treatment modality in addition to another treatment modality, for example, a treatment regimen that includes administration of a PD-1 axis binding antagonist (e.g., an anti-PD-L1 antibody, e.g., atezolizumab) and a platinum-based chemotherapy (e.g., cisplatin or carboplatin and gemcitabine). As such, “in combination with” refers to administration of one treatment modality before, during, or after administration of the other treatment modality to the patient.

A drug that is administered “concurrently” with one or more other drugs is administered during the same treatment cycle, on the same day of treatment, as the one or more other drugs, and, optionally, at the same time as the one or more other drugs. For instance, for cancer therapies given every 3 weeks, the concurrently administered drugs are each administered on day 1 of a 3 week cycle.

The term “detection” includes any means of detecting, including direct and indirect detection.

The term “biomarker” as used herein refers to an indicator, e.g., predictive, diagnostic, and/or prognostic, which can be detected in a sample, for example, PD-L1. The biomarker may serve as an indicator of a particular subtype of a disease or disorder (e.g., cancer) characterized by certain, molecular, pathological, histological, and/or clinical features. In some embodiments, a biomarker is a gene. Biomarkers include, but are not limited to, polynucleotides (e.g., DNA and/or RNA), polynucleotide copy number alterations (e.g., DNA copy numbers), polypeptides, polypeptide and polynucleotide modifications (e.g., post-translational modifications), carbohydrates, and/or glycolipid-based molecular markers.

The “amount” or “level” of a biomarker associated with an increased clinical benefit to an individual is a detectable level in a biological sample. These can be measured by methods known to one skilled in the art and also disclosed herein. The expression level or amount of biomarker assessed can be used to determine the response to the treatment.

The terms “level of expression” or “expression level” in general are used interchangeably and generally refer to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Increased expression,” “increased expression level,” “increased levels,” “elevated expression,” “elevated expression levels,” or “elevated levels” refers to an increased expression or increased levels of a biomarker in an individual relative to a control, such as an individual or individuals who are not suffering from the disease or disorder (e.g., cancer) or an internal control (e.g., a housekeeping biomarker). In some examples, the control is a reference expression level.

“Decreased expression,” “decreased expression level,” “decreased levels,” “reduced expression,” “reduced expression levels,” or “reduced levels” refers to a decrease expression or decreased levels of a biomarker in an individual relative to a control, such as an individual or individuals who are not suffering from the disease or disorder (e.g., cancer) or an internal control (e.g., a housekeeping biomarker). In some embodiments, reduced expression is little or no expression. In some examples, the control is a reference expression level.

The term “housekeeping biomarker” refers to a biomarker or group of biomarkers (e.g., polynucleotides and/or polypeptides) which are typically similarly present in all cell types. In some embodiments, the housekeeping biomarker is a “housekeeping gene.” A “housekeeping gene” refers herein to a gene or group of genes which encode proteins whose activities are essential for the maintenance of cell function and which are typically similarly present in all cell types.

The term “diagnosis” is used herein to refer to the identification or classification of a molecular or pathological state, disease or condition (e.g., cancer (e.g., bladder cancer (e.g., UC, including locally advanced or metastatic UC))). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)).

The term “sample,” as used herein, refers to a composition that is obtained or derived from a subject and/or patient of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In some examples, the sample is a tumor sample (e.g., a tumor tissue sample).

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor (e.g., a bladder tumor, e.g., a UC tumor (e.g., a locally advanced or metastatic UC tumor)) or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like. In some examples, the tissue sample is a tumor tissue sample.

A “tumor-infiltrating immune cell,” as used herein, refers to any immune cell present in a tumor or a sample thereof. Tumor-infiltrating immune cells include, but are not limited to, intratumoral immune cells, peritumoral immune cells, other tumor stroma cells (e.g., fibroblasts), or any combination thereof. Such tumor-infiltrating immune cells can be, for example, T lymphocytes (such as CD8+ T lymphocytes and/or CD4+ T lymphocytes), B lymphocytes, or other bone marrow-lineage cells, including granulocytes (e.g., neutrophils, eosinophils, and basophils), monocytes, macrophages, dendritic cells (e.g., interdigitating dendritic cells), histiocytes, and natural killer cells. In some examples, the tumor-infiltrating immune cell may include a dendritic cell (e.g., a dendritic cell that is positive for DC-LAMP).

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, the reference sample, reference cell, reference tissue, control sample, control cell, or control tissue may be healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

For the purposes herein a “section” of a tissue sample is meant a single part or piece of a tissue sample, for example, a thin slice of tissue or cells cut from a tissue sample (e.g., a tumor sample). It is to be understood that multiple sections of tissue samples may be taken and subjected to analysis, provided that it is understood that the same section of tissue sample may be analyzed at both morphological and molecular levels, or analyzed with respect to polypeptides (e.g., by immunohistochemistry) and/or polynucleotides (e.g., by in situ hybridization). In some examples, the sections may be consecutive sections. In other examples, the sections may be non-consecutive sections.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

The phrase “based on” when used herein means that the information about one or more biomarkers is used to inform a treatment decision, information provided on a package insert, or marketing/promotional guidance, and the like.

As used herein, the term “Combined Positive Score” or “CPS” refers to the number of PD-L1 staining cells (e.g., tumor cells, lymphocytes, or macrophages) divided by the total number of viable tumor cells, multiplied by 100, in the context of an AHC assay (e.g., an IHC assay), e.g., an IHC assay staining for PD-L1 using the antibody SP142, SP263, 22C3, or 28-8. In one example, a CPS may be calculated using a PD-L1 IHC assay comprising the Dako 22C3 anti-PD-L1 diagnostic antibody, e.g., the PD-L1 IHC 22C3 PHARMDX assay (Dako) according to the formula above. In other examples, a CPS may be calculated using another anti-PD-L1 diagnostic antibody, e.g., SP263 or 28-8. In some examples, a sample (e.g., a tumor sample) may be considered to have PD-L1 expression if CPS is ≥1 or ≥10. In particular examples, a sample (e.g., a tumor sample) may be considered to have PD-L1 expression if CPS is ≥10.

As used herein, a “PD-L1-positive tumor cell fraction” is the percentage of viable tumor cells showing partial or complete membrane staining (exclusive of cytoplasmic staining) at any intensity relative to all viable tumor cells present in a sample, following staining of the sample in the context of an AHC assay (e.g., an IHC assay), e.g., an IHC assay staining for PD-L1 using the antibody SP142, SP263, 22C3, or 28-8. Accordingly, a PD-L1-positive tumor cell fraction may be calculated using the PD-L1 IHC SP263 (Ventana) assay, for example, by the formula PD-L1-positive tumor cell fraction=(number of PD-L1-positive tumor cells)/(total number of PD-L1-positive and PD-L1 negative tumor cells), wherein PD-L1 cytoplasmic staining of tumor cells and all non-tumor cells (e.g., tumor-infiltrating immune cells, normal cells, necrotic cells, and debris) are excluded from evaluation and scoring. It will be appreciated that any given diagnostic PD-L1 antibody may correspond with a particular IHC assay protocol and/or scoring terminology that can be used to derive a PD-L1-positive tumor cell fraction. For example, a PD-L1-positive tumor cell fraction can be derived from a tumor cell sample stained with SP263, 22C3, SP142, or 28-8 using OPTIVIEW® detection on Benchmark ULTRA®, EnVision Flex on AutostainerLink 48, OPTIVIEW® detection and amplification on Benchmark ULTRA®, or EnVision Flex on AutostainerLink 48, respectively. In another example, a PD-L1-positive tumor cell fraction may be calculated using the PD-L1 IHC 22C3 PHARMDX assay (Dako) according to the formula above. As used herein, the terms PD-L1-positive tumor cell fraction and “tumor proportion score” (TPS) are used interchangeably.

For the purposes herein, “anti-PD-L1 diagnostic antibody” refers to an antibody that is capable of binding PD-L1 with sufficient affinity such that the antibody is useful as a diagnostic agent for detecting the presence and/or expression level of PD-L1 in a biological sample (e.g., a tumor sample) obtained from a patient. In one embodiment, the extent of binding of an anti-PD-L1 diagnostic antibody to an unrelated, non-PD-L1 protein is less than about 10% of the binding of the antibody to PD-L1 as measured, e.g., by a radioimmunoassay (RIA). In certain embodiments, an antibody that binds to PD-L1 has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-PD-L1 diagnostic antibody binds to an epitope of PD-L1 that is conserved among PD-L1 from different species. Exemplary anti-PD-L1 diagnostic antibodies include, but are not limited to, SP142 (Ventana), SP263 (Ventana), 22C3 (Dako), 28-8 (Dako), E1L3N (Cell Signaling Technology), 4059 (ProSci, Inc.), h5H1 (Advanced Cell Diagnostics), and 9A11. In some examples, the anti-PD-L1 diagnostic antibody is SP142. In other examples, the anti-PD-L1 diagnostic antibody is SP263, 22C3, or 28-8.

As used herein, “VENTANA SP142” or “SP142” refers to an anti-PD-L1 diagnostic antibody described in U.S. Pat. No. 10,689,445, which is incorporated by reference herein in its entirety. The VENTANA PD-L1 (SP142) assay is commercially available. The heavy and light chain variable region sequences of the SP142 antibody are as follows (the hypervariable sequences HVR-H1, HVR-H2, HVR-H3, HVR-L1, HVR-L2, and HVR-L3 are indicated by underlined and italicized text).

```
Heavy chain variable region:
(SEQ ID NO: 11)
QSLEESGGRLVKPDETLTITCTVSGIDLSSNGLTWVRQAPGEGLEWIG
                                                          HVR-H1
TINKDASAYYASWAKGRLTISKPSSTKVDLKITSPTTEDTATYFCGR
            HVR-H2
IAFKTGTSIWGPGTLVTVSS
  HVR-H3
Light chain variable region:
(SEQ ID NO: 12)
AIVMTQTPSPVSAAVGGTVTINCQASESVYSNNYLSWFQQKPGQPPKL
                                                      HVR-L1
LIYLASTLASGVPSRFKGSGSGTQFTLTISGVQCDDAATYYC
        HVR-L2
IGGKSSSTDGNAFGGGTEVVVR.
        HVR-L3
```

As used herein, “VENTANA SP263” or “SP263” refers to an anti-PD-L1 diagnostic antibody described in U.S. Pat. No. 10,775,383 and WO 2015/181342, which are incorporated by reference herein in their entirety. The VENTANA PD-L1 (SP263) assay is commercially available. Amino acid sequences of the VENTANA SP263 anti-PD-L1 diagnostic antibody are shown, e.g., in U.S. Pat. No. 10,775,383 (see, e.g., Example 1 and Table 1).

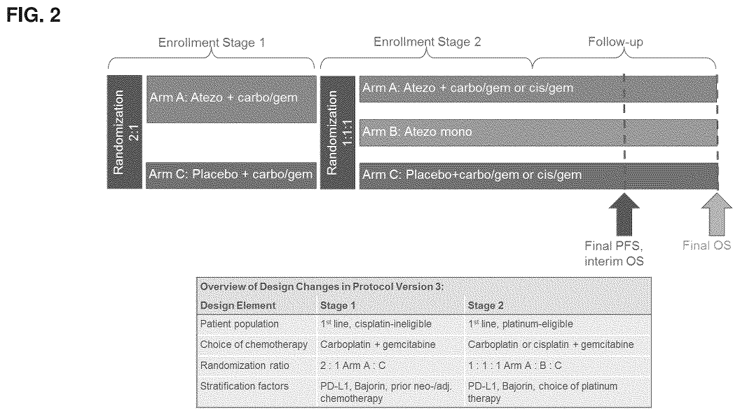

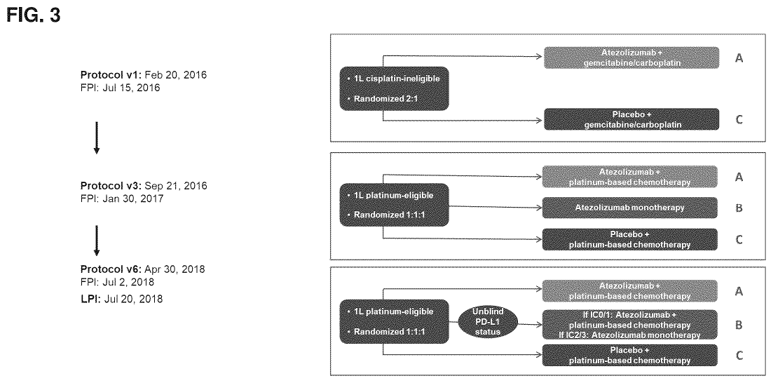

As used herein, “Dako 22C3” or “22C3” refers to a commercially available anti-PD-L1 diagnostic antibody. The Dako 22C3 anti-PD-L1 diagnostic antibody is described in U.S. Pat. No. 9,709,568 and WO 2014/100079, which are incorporated by reference herein in their entirety. The PD-L1 IHC 22C3 PHARMDX assay is commercially available (Agilent Dako). Amino acid sequences of the Dako 22C3 anti-PD-L1 diagnostic antibody sequences are shown, e.g., in U.S. Pat. No. 9,709,568 (see, e.g., FIGS. 2 and 3 and Table 2 of U.S. Pat. No. 9,709,568).

As used herein, “28-8” refers to a commercially available anti-PD-L1 diagnostic antibody. The 28-8 anti-PD-L1 diagnostic antibody is described in U.S. Pat. No. 9,212,224 and WO 2013/173223, which are incorporated by reference herein in their entirety. The PD-L1 IHC 28-8 PHARMDX assay is commercially available (Agilent Dako). Amino acid sequences of the 28-8 anti-PD-L1 diagnostic antibody are shown, e.g., in U.S. Pat. No. 9,212,224. For example, U.S. Pat. No. 9,212,224 describes that the heavy and light chain variable region amino acid sequences of 28-8 are set forth in SEQ ID NO: 35 and SEQ ID NO: 36, respectively, of U.S. Pat. No. 9,212,224.

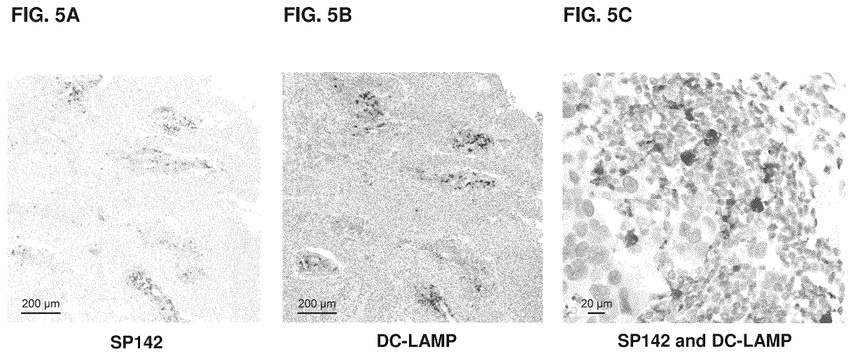

As used herein, the term “immune-directed PD-L1 assay” refers to any affinity histochemical (AHC) assay (e.g., any IHC assay) specific for human PD-L1 protein that has been designed to highlight immune cell expression of PD-L1, for example, by preferentially staining PD-L1-expressing immune cells versus PD-L1-expressing tumor cells. The highlighting of the immune cells may be a result of (a) inherent antibody specificity for immune-expressed PD-L1 versus expression by other cell types; (b) careful selection of staining conditions, such as antigen retrieval process, antibody diluent selection, buffer selection, detection system, labeling time and temperature, etc.; or (c) a combination of (a) and (b). An example of a commercially available immune-directed PD-L1 assay is the VENTANA PD-L1 (SP142) Assay (“SP142 Assay”). The SP142 Assay is an affinity histochemical assay that uses: (a) a PD-L1 rabbit monoclonal antibody (clone SP142, see U.S. Pat. No. 10,689,445); (b) an automated IHC/ISH staining platform (BENCHMARK IHC/ISH staining platform (Roche)); and (c) a tyramide-amplified 3,3′-diaminobenzedine (DAB)-based detection system (OPTIVIEW DAB IHC detection kit with OPTIVIEW Amplification kit (Roche)). As illustrated at FIGS. 5A-5C, the SP142 assay preferentially stains immune cells in tumor sections, especially dendritic cells. The SP142 assay is also capable of staining tumor cells, but antibody selection and assay conditions were optimized to emphasize immune cell staining.

As used herein, the term “immune-agnostic PD-L1 assay” is any affinity histochemical assay specific for human PD-L1 protein that is not an immune-directed PD-L1 assay. Exemplary commercially available immune-agnostic PD-L1 assays include the PD-L1 IHC 22C3 PHARMDX assay (Agilent) (“22C3 Assay”), the VENTANA PD-L1 (SP263) Assay (Roche) (“SP263 Assay”), and the PD-L1 IHC 28-8 PHARMDX assay (Agilent) (“28-8 Assay”).

### Therapeutic and Diagnostic Methods and Compositions for Bladder Cancer

Provided herein are methods for treating or delaying progression of bladder cancer (e.g., UC, including locally advanced or metastatic UC) in a subject comprising administering to the subject a treatment regimen comprising an effective amount of a PD-1 axis binding antagonist (e.g., an anti-PD-L1 antibody (e.g., atezolizumab)). Also provided herein are methods for treating or delaying progression of bladder cancer (e.g., UC, including locally advanced or metastatic UC) in a subject comprising administering to the subject a treatment regimen comprising an effective amount of a PD-1 axis binding antagonist (e.g., an anti-PD-L1 antibody (e.g., atezolizumab)) and a platinum-based chemotherapy (e.g., cisplatin or carboplatin and gemcitabine). In some embodiments, the treatment results in a response in the subject after treatment. For example, in some embodiments, the treatment increases the subject's likelihood of having an objective response (e.g., a complete response (CR)), extends the subject's progression-free survival (PFS), extends the subject's overall survival (OS), and/or extends the subject's duration of response (DOR), for example, as compared to a reference treatment, e.g., treatment without the PD-1 axis binding antagonist or treatment with a platinum-based chemotherapy without the PD-1 axis binding antagonist. Also provided herein are methods of enhancing immune function in a subject having a bladder cancer (e.g., UC, including locally advanced or metastatic UC) comprising administering to the subject a treatment regimen comprising an effective amount of a PD-1 axis binding antagonist (e.g., an anti-PD-L1 antibody (e.g., atezolizumab)). Further provided herein are methods of enhancing immune function in a subject having a bladder cancer (e.g., UC, including locally advanced or metastatic UC) comprising administering to the subject a treatment regimen comprising an effective amount of a PD-1 axis binding antagonist (e.g., an anti-PD-L1 antibody (e.g., atezolizumab)) and a platinum-based chemotherapy (e.g., cisplatin or carboplatin and gemcitabine). Also provided are methods of identifying a tumor likely to respond to a PD-1 axis binding antagonist. Further provided are methods of patient identification and/or selection, e.g., for treatment with a PD-1 axis binding antagonist. Further provided are methods of stratifying a tumor. Any of the PD-1 axis binding antagonists and/or the platinum-based chemotherapies known in the art or described herein may be used in the methods. 35

In another aspect, provided herein is a method of identifying a tumor likely to respond to a PD-1 axis binding antagonist, the method comprising: (a) staining a first portion of the tumor with an immune-directed PD-L1 assay to obtain a first stained sample; (b) generating a first score by applying a first scoring algorithm to the first stained sample; (c) staining a second portion of the tumor with an immune-agnostic PD-L1 assay to obtain a second stained sample; (d) generating a second score by applying a second scoring algorithm the second stained sample; and (e) comparing the first score to a first cutoff and the second score to a second cutoff, wherein the tumor is likely to respond to the PD-1 axis binding antagonist when both the first score meets or exceeds the first cutoff and the second score meets or exceeds the second cutoff.

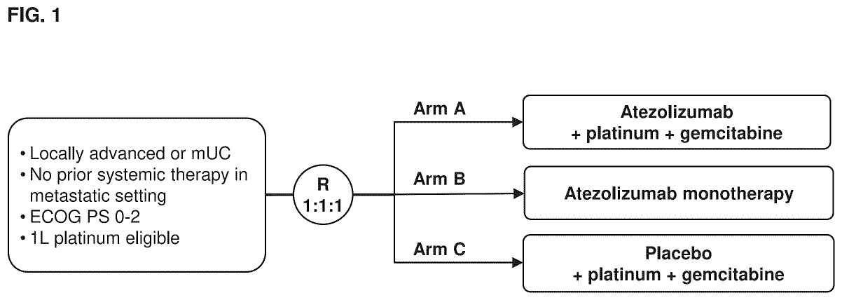

Any suitable first scoring algorithm and/or second scoring algorithm may be used. For example, the first scoring algorithm may be the immune cell scoring algorithm set forth in Table 2 herein, e.g., as used in an SP142 Assay. In some examples the second scoring algorithm may be a Combined Positive Score (CPS), e.g., as used in a 22C3 Assay. It is to be understood that a CPS may be determined using other PD-L1 AHC assays (e.g., other PD-L1 IHC assays), e.g., an IHC assay comprising use of VENTANA SP263 or 28-8. Other scoring algorithms for PD-L1 assays are known in the art, e.g., TPS, percent of tumor cells (TC), and the tumor cell scoring algorithm set forth in Table 3 herein. A description of different exemplary scoring algorithms for PD-L1 assays that may be used is shown in FIG. 1 of Zajac et al. Diagnostic Pathology.

Any suitable first cutoff and second cutoff may be used. For example, in some examples, the first cutoff is IC ≥5%, e.g., as described in Table 2 herein. In some examples, the second cutoff is CPS ≥1 or CPS ≥10. In some examples, the second cutoff is CPS ≥10.

In some aspects, the immune-directed PD-L1 assay has: (i) at least an 80% overall percent agreement (OPA) with an SP142 Assay using the first scoring algorithm at the first cutoff value; (ii) at least an 80% positive percent agreement (PPA) with an SP142 Assay using the first scoring algorithm at the first cutoff value; (iii) at least an 80% negative percent agreement (NPA) with an SP142 Assay using the first scoring algorithm at the first cutoff value; (iv) at least an 80% PPA and at least an 80% NPA with an SP142 Assay using the first scoring algorithm at the first cutoff value; (v) at least an 80% PPA and at least an 80% OPA with an SP142 Assay using the first scoring algorithm at the first cutoff value; (vi) at least an 80% NPA and at least an 80% OPA with an SP142 Assay using the first scoring algorithm at the first cutoff value; and/or (vii) at least an 80% OPA, at least an 80% PPA, and at least an 80% NPA with an SP142 Assay using the first scoring algorithm at the first cutoff value.

In some aspects, the immune-agnostic PD-L1 assay has: (i) at least an 80% OPA with an 22C3 Assay using the second scoring algorithm at the second cutoff value; (ii) at least an 80% PPA with an 22C3 Assay using the second scoring algorithm at the second cutoff value; (iii) at least an 80% NPA with an 22C3 Assay using the second scoring algorithm at the second cutoff value; (iv) at least an 80% PPA and at least an 80% NPA with an 22C3 Assay using the second scoring algorithm at the second cutoff value; (v) at least an 80% PPA and at least an 80% OPA with an 22C3 Assay using the second scoring algorithm at the second cutoff value; (vi) at least an 80% NPA and at least an 80% OPA with an 22C3 Assay using the second scoring algorithm at the second cutoff value; and/or (vii) at least an 80% OPA, at least an 80% PPA, and at least an 80% NPA with an 22C3 Assay using the second scoring algorithm at the second cutoff value.

In another aspect, provided herein is a method of stratifying a tumor having a score with an immune-agnostic PD-L1 assay that exceeds a pre-determined cutoff, the method comprising: (a) staining a portion of the tumor with an immune-directed PD-L1 assay to obtain a stained sample; (b) generating a score by applying a scoring algorithm to the stained sample; and (c) comparing the score to a first cutoff, wherein the tumor is likely to respond to a PD-1 axis binding antagonist when both the first score meets or exceeds the first cutoff.

In another aspect, provided herein is a method of stratifying a CPS ≥10% tumor as determined by a 22C3 assay, the method comprising: (a) staining a portion of the tumor with an immune-directed PD-L1 assay to obtain a stained sample; (b) generating a score by applying a scoring algorithm to the stained sample; and (c) comparing the score to a first cutoff and the second score to a second cutoff, wherein the tumor is likely to respond to the PD-1 axis binding antagonist when both the first score meets or exceeds the first cutoff and the second score meets or exceeds the second cutoff.

In another aspect, provided herein is a method of stratifying a CPS ≥10% tumor as determined by an SP263 assay, the method comprising: (a) staining a portion of the tumor with an immune-directed PD-L1 assay to obtain a stained sample; (b) generating a score by applying a scoring algorithm to the stained sample; and (c) comparing the score to a first cutoff and the second score to a second cutoff, wherein the tumor is likely to respond to the PD-1 axis binding antagonist when both the first score meets or exceeds the first cutoff and the second score meets or exceeds the second cutoff.

In another aspect, provided herein is a method of stratifying a CPS ≥10% tumor as determined by a 28-8 assay, the method comprising: (a) staining a portion of the tumor with an immune-directed PD-L1 assay to obtain a stained sample; (b) generating a score by applying a scoring algorithm to the stained sample; and (c) comparing the score to a first cutoff and the second score to a second cutoff, wherein the tumor is likely to respond to the PD-1 axis binding antagonist when both the first score meets or exceeds the first cutoff and the second score meets or exceeds the second cutoff.

Any suitable scoring algorithm may be used. For example, the scoring algorithm may be the immune cell scoring algorithm set forth in Table 2 herein, e.g., as used in an SP142 Assay. In some examples the scoring algorithm may be a Combined Positive Score (CPS), e.g., as used in a 22C3 Assay. It is to be understood that a CPS may be determined using other PD-L1 AHC assays (e.g., other PD-L1 IHC assays), e.g., an IHC assay comprising use of VENTANA SP263 or 28-8. Other scoring algorithms for PD-L1 assays are known in the art, e.g., TPS, percent of tumor cells (TC), and the tumor cell scoring algorithm set forth in Table 3 herein. A description of different exemplary scoring algorithms for PD-L1 assays that may be used is shown in FIG. 1 of Zajac et al. Diagnostic Pathology.

Any suitable cutoff may be used. For example, in some examples, the cutoff is IC ≥5%, e.g., as described in Table 2 herein (e.g., IC2/3). In some examples, the cutoff is CPS ≥1 or CPS ≥10. In some examples, the cutoff is CPS ≥10.

The tumor may be of any suitable cancer type (e.g., bladder cancer (e.g., UC, including metastatic UC (mUC); muscle-invasive bladder cancer (MIBC), and non-muscle-invasive bladder cancer (NMIBC)); kidney or renal cancer (e.g., renal cell carcinoma (RCC)); lung cancer, including small-cell lung cancer, non-small cell lung cancer, adenocarcinoma of the lung, and squamous carcinoma of the lung; cancer of the urinary tract; breast cancer (e.g., HER2+ breast cancer and triple-negative breast cancer (TNBC), which are estrogen receptors (ER−), progesterone receptors (PR−), and HER2 (HER2−) negative); prostate cancer, such as castration-resistant prostate cancer (CRPC); cancer of the peritoneum; hepatocellular cancer; gastric or stomach cancer, including gastrointestinal cancer and gastrointestinal stromal cancer; pancreatic cancer (e.g., pancreatic ductal adenocarcinoma (PDAC)); glioblastoma; cervical cancer; ovarian cancer; liver cancer (e.g., hepatocellular carcinoma (HCC)); hepatoma; colon cancer; rectal cancer; colorectal cancer; endometrial or uterine carcinoma; salivary gland carcinoma; prostate cancer; vulval cancer; thyroid cancer; hepatic carcinoma; anal carcinoma; penile carcinoma; melanoma, including superficial spreading melanoma, lentigo malignant melanoma, acral lentiginous melanomas, and nodular melanomas; multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases). In some aspects, the tumor is a bladder cancer, e.g., a locally advanced or metastatic UC.

In another aspect, provided herein is a method of treating a patient suffering from a cancer who has been identified or stratified according to any of the preceding methods, the method comprising administering to the patient a treatment regimen comprising a PD-1 axis binding antagonist (e.g., an anti-PD-L1 antibody (e.g., atezolizumab)).

In one example, provided herein is a method of treating a cancer (e.g., a bladder cancer (e.g., a locally advanced or metastatic UC)) in a patient in need thereof, the method comprising administering to the patient a treatment regimen comprising a PD-1 axis binding antagonist (e.g., an anti-PD-L1 antibody (e.g., atezolizumab)), wherein a tumor sample obtained from the patient has been determined to have: a detectable expression level of PD-L1 in tumor-infiltrating immune cells using an immune-directed PD-L1 assay; and a detectable expression level of PD-L1 using with an immune-agnostic PD-L1 assay, thereby identifying the patient as one who may benefit from the treatment regimen comprising the PD-1 axis binding antagonist.

In another example, provided herein is a method of treating a cancer (e.g., a bladder cancer (e.g., a locally advanced or metastatic UC)) in a patient in need thereof, the method comprising: (a) determining that a tumor sample obtained from the patient has: a detectable expression level of PD-L1 in tumor-infiltrating immune cells using an immune-directed PD-L1 assay; and a detectable expression level of PD-L1 using with an immune-agnostic PD-L1 assay, thereby identifying the patient as one who may benefit from a treatment regimen comprising a PD-1 axis binding antagonist (e.g., an anti-PD-L1 antibody (e.g., atezolizumab)); and (b) administering the treatment regimen comprising the PD-1 axis binding antagonist to the patient identified in step (a) as one who may benefit from the treatment regimen comprising the PD-1 axis binding antagonist.

In one example, provided herein is a method of enhancing immune function in a patient having a cancer (e.g., a bladder cancer (e.g., a locally advanced or metastatic UC)), the method comprising administering to the patient a treatment regimen comprising a PD-1 axis binding antagonist (e.g., an anti-PD-L1 antibody (e.g., atezolizumab)), wherein a tumor sample obtained from the patient has been determined to have: a detectable expression level of PD-L1 in tumor-infiltrating immune cells using an immune-directed PD-L1 assay; and a detectable expression level of PD-L1 using with an immune-agnostic PD-L1 assay, thereby identifying the patient as one who may benefit from the treatment regimen comprising the PD-1 axis binding antagonist.

In another example, provided herein is a method of enhancing immune function in a patient having a cancer (e.g., a bladder cancer (e.g., a locally advanced or metastatic UC)), the method comprising: (a) determining that a tumor sample obtained from the patient has: a detectable expression level of PD-L1 in tumor-infiltrating immune cells using an immune-directed PD-L1 assay; and a detectable expression level of PD-L1 using with an immune-agnostic PD-L1 assay, thereby identifying the patient as one who may benefit from a treatment regimen comprising a PD-1 axis binding antagonist (e.g., an anti-PD-L1 antibody (e.g., atezolizumab)); and (b) administering the treatment regimen comprising the PD-1 axis binding antagonist to the patient identified in step (a) as one who may benefit from the treatment regimen comprising the PD-1 axis binding antagonist.

In another example, provided herein is a PD-1 axis binding antagonist (e.g., an anti-PD-L1 antibody (e.g., atezolizumab)) for use in treatment of a cancer (e.g., a bladder cancer (e.g., a locally advanced or metastatic UC)) in a patient in need thereof, the treatment comprising administration to the patient of a treatment regimen comprising a PD-1 axis binding antagonist, wherein a tumor sample obtained from the patient has been determined to have: a detectable expression level of PD-L1 in tumor-infiltrating immune cells using an immune-directed PD-L1 assay; and a detectable expression level of PD-L1 using with an immune-agnostic PD-L1 assay, thereby identifying the patient as one who may benefit from the treatment regimen comprising the PD-1 axis binding antagonist.

In another example, provided herein is a PD-1 axis binding antagonist (e.g., an anti-PD-L1 antibody (e.g., atezolizumab)) for use in a method of treating a cancer (e.g., a bladder cancer (e.g., a locally advanced or metastatic UC)) in a patient in need thereof, the method comprising: (a) determining that a tumor sample obtained from the patient has: a detectable expression level of PD-L1 in tumor-infiltrating immune cells using an immune-directed PD-L1 assay; and a detectable expression level of PD-L1 using with an immune-agnostic PD-L1 assay, thereby identifying the patient as one who may benefit from a treatment regimen comprising a PD-1 axis binding antagonist; and (b) administering the treatment regimen comprising the anti-PD-L1 antibody to the patient identified in step (a) as one who may benefit from the treatment regimen comprising the PD-1 axis binding antagonist.

In another example, provided herein is a PD-1 axis binding antagonist (e.g., an anti-PD-L1 antibody (e.g., atezolizumab)) for use in enhancing immune function in a patient having a cancer (e.g., a bladder cancer (e.g., a locally advanced or metastatic UC)), the treatment comprising administration to the patient of a treatment regimen comprising a PD-1 axis binding antagonist, wherein a tumor sample obtained from the patient has been determined to have: a detectable expression level of PD-L1 in tumor-infiltrating immune cells using an immune-directed PD-L1 assay; and a detectable expression level of PD-L1 using with an immune-agnostic PD-L1 assay, thereby identifying the patient as one who may benefit from the treatment regimen comprising the PD-1 axis binding antagonist.

In another example, provided herein is a PD-1 axis binding antagonist (e.g., an anti-PD-L1 antibody (e.g., atezolizumab)) for use in a method of method of enhancing immune function in a patient having a cancer (e.g., a bladder cancer (e.g., a locally advanced or metastatic UC)), the method comprising: (a) determining that a tumor sample obtained from the patient has: a detectable expression level of PD-L1 in tumor-infiltrating immune cells using an immune-directed PD-L1 assay; and a detectable expression level of PD-L1 using with an immune-agnostic PD-L1 assay, thereby identifying the patient as one who may benefit from a treatment regimen comprising a PD-1 axis binding antagonist; and (b) administering the treatment regimen comprising the anti-PD-L1 antibody to the patient identified in step (a) as one who may benefit from the treatment regimen comprising the PD-1 axis binding antagonist.

In another example, provided herein is a method of selecting a therapy for treating a cancer (e.g., a bladder cancer (e.g., a locally advanced or metastatic UC)) in a patient in need thereof, the method comprising: (a) determining that a tumor sample obtained from the patient has: a detectable expression level of PD-L1 in tumor-infiltrating immune cells using an immune-directed PD-L1 assay; and a detectable expression level of PD-L1 using with an immune-agnostic PD-L1 assay, thereby identifying the patient as one who may benefit from a treatment regimen comprising a PD-1 axis binding antagonist (e.g., an anti-PD-L1 antibody (e.g., atezolizumab)); and (b) selecting a treatment regimen comprising the PD-1 axis binding antagonist for the patient identified in step (a) as one who may benefit from the treatment regimen comprising the PD-1 axis binding antagonist.

In another example, provided herein is a method of identifying a patient having a cancer (e.g., a bladder cancer (e.g., a locally advanced or metastatic UC)) who may benefit from a treatment regimen comprising a PD-1 axis binding antagonist (e.g., an anti-PD-L1 antibody (e.g., atezolizumab)), the method comprising: determining that a tumor sample obtained from the patient has: a detectable expression level of PD-L1 in tumor-infiltrating immune cells using an immune-directed PD-L1 assay; and a detectable expression level of PD-L1 using with an immune-agnostic PD-L1 assay, thereby identifying the patient as one who may benefit from a treatment regimen comprising a PD-1 axis binding antagonist.

In some examples, the immune-directed PD-L1 assay is an AHC assay (e.g., an IHC assay).

In some examples, the immune-agnostic PD-L1 assay is an AHC assay (e.g., an IHC assay).

The cancer may be any suitable type of cancer (e.g., bladder cancer (e.g., UC, including mUC; MIBC, and NMIBC); kidney or renal cancer (e.g., RCC); lung cancer, including small-cell lung cancer, non-small cell lung cancer, adenocarcinoma of the lung, and squamous carcinoma of the lung; cancer of the urinary tract; breast cancer (e.g., HER2+ breast cancer and TNBC, which are ER−, PR−, and HER2 HER2−); prostate cancer, such as CRPC; cancer of the peritoneum; hepatocellular cancer; gastric or stomach cancer, including gastrointestinal cancer and gastrointestinal stromal cancer; pancreatic cancer (e.g., PDAC); glioblastoma; cervical cancer; ovarian cancer; liver cancer (e.g., HCC); hepatoma; colon cancer; rectal cancer; colorectal cancer; endometrial or uterine carcinoma; salivary gland carcinoma; prostate cancer; vulval cancer; thyroid cancer; hepatic carcinoma; anal carcinoma; penile carcinoma; melanoma, including superficial spreading melanoma, lentigo malignant melanoma, acral lentiginous melanomas, and nodular melanomas; multiple myeloma and B-cell lymphoma (including low grade/follicular NHL; SL NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); CLL; ALL; AML; hairy cell leukemia; CML; PTLD; and MDS, as well as abnormal vascular proliferation associated with phakomatoses, edema (such as that associated with brain tumors), Meigs' syndrome, brain cancer, head and neck cancer, and associated metastases). In some examples, the cancer is a bladder cancer (e.g., a locally advanced or metastatic UC).

In one example, provided herein is a method of treating a bladder cancer (e.g., a locally advanced or metastatic UC) in a patient in need thereof, the method comprising administering to the patient a treatment regimen comprising a PD-1 axis binding antagonist (e.g., an anti-PD-L1 antibody (e.g., atezolizumab)), wherein a tumor sample obtained from the patient has been determined to have: a detectable expression level of PD-L1 in tumor-infiltrating immune cells using a PD-L1 immunohistochemical (IHC) assay comprising the VENTANA SP142 anti-PD-L1 diagnostic antibody; and a detectable expression level of PD-L1 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from the treatment regimen comprising the PD-1 axis binding antagonist.

In another example, provided herein is a method of treating a bladder cancer (e.g., a locally advanced or metastatic UC) in a patient in need thereof, the method comprising: (a) determining that a tumor sample obtained from the patient has: a detectable expression level of PD-L1 in tumor-infiltrating immune cells using a PD-L1 IHC assay comprising the VENTANA SP142 anti-PD-L1 diagnostic antibody; and a detectable expression level of PD-L1 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising a PD-1 axis binding antagonist (e.g., an anti-PD-L1 antibody (e.g., atezolizumab)); and (b) administering the treatment regimen comprising the PD-1 axis binding antagonist to the patient identified in step (a) as one who may benefit from the treatment regimen comprising the PD-1 axis binding antagonist.

In one example, provided herein is a method of enhancing immune function in a patient having a bladder cancer (e.g., a locally advanced or metastatic UC), the method comprising administering to the patient a treatment regimen comprising a PD-1 axis binding antagonist (e.g., an anti-PD-L1 antibody (e.g., atezolizumab)), wherein a tumor sample obtained from the patient has been determined to have: a detectable expression level of PD-L1 in tumor-infiltrating immune cells using a PD-L1 IHC assay comprising the VENTANA SP142 anti-PD-L1 diagnostic antibody; and a detectable expression level of PD-L1 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from the treatment regimen comprising the PD-1 axis binding antagonist.

In another example, provided herein is a method of enhancing immune function in a patient having a bladder cancer (e.g., a locally advanced or metastatic UC), the method comprising: (a) determining that a tumor sample obtained from the patient has: a detectable expression level of PD-L1 in tumor-infiltrating immune cells using a PD-L1 IHC assay comprising the VENTANA SP142 anti-PD-L1 diagnostic antibody; and a detectable expression level of PD-L1 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising a PD-1 axis binding antagonist (e.g., an anti-PD-L1 antibody (e.g., atezolizumab)); and (b) administering the treatment regimen comprising the PD-1 axis binding antagonist to the patient identified in step (a) as one who may benefit from the treatment regimen comprising the PD-1 axis binding antagonist.

In another example, provided herein is a PD-1 axis binding antagonist (e.g., an anti-PD-L1 antibody (e.g., atezolizumab)) for use in treatment of a bladder cancer (e.g., a locally advanced or metastatic UC) in a patient in need thereof, the treatment comprising administration to the patient of a treatment regimen comprising a PD-1 axis binding antagonist, wherein a tumor sample obtained from the patient has been determined to have: a detectable expression level of PD-L1 in tumor-infiltrating immune cells using a PD-L1 IHC assay comprising the VENTANA SP142 anti-PD-L1 diagnostic antibody; and a detectable expression level of PD-L1 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from the treatment regimen comprising the PD-1 axis binding antagonist.

In another example, provided herein is a PD-1 axis binding antagonist (e.g., an anti-PD-L1 antibody (e.g., atezolizumab)) for use in a method of treating a bladder cancer (e.g., a locally advanced or metastatic UC) in a patient in need thereof, the method comprising: (a) determining that a tumor sample obtained from the patient has: a detectable expression level of PD-L1 in tumor-infiltrating immune cells using a PD-L1 IHC assay comprising the VENTANA SP142 anti-PD-L1 diagnostic antibody; and a detectable expression level of PD-L1 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising a PD-1 axis binding antagonist; and (b) administering the treatment regimen comprising the anti-PD-L1 antibody to the patient identified in step (a) as one who may benefit from the treatment regimen comprising the PD-1 axis binding antagonist.

In another example, provided herein is a PD-1 axis binding antagonist (e.g., an anti-PD-L1 antibody (e.g., atezolizumab)) for use in enhancing immune function in a patient having a bladder cancer (e.g., a locally advanced or metastatic UC), the treatment comprising administration to the patient of a treatment regimen comprising a PD-1 axis binding antagonist, wherein a tumor sample obtained from the patient has been determined to have: a detectable expression level of PD-L1 in tumor-infiltrating immune cells using a PD-L1 IHC assay comprising the VENTANA SP142 anti-PD-L1 diagnostic antibody; and a detectable expression level of PD-L1 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from the treatment regimen comprising the PD-1 axis binding antagonist.

In another example, provided herein is a PD-1 axis binding antagonist (e.g., an anti-PD-L1 antibody (e.g., atezolizumab)) for use in a method of method of enhancing immune function in a patient having a bladder cancer (e.g., a locally advanced or metastatic UC), the method comprising: (a) determining that a tumor sample obtained from the patient has: a detectable expression level of PD-L1 in tumor-infiltrating immune cells using a PD-L1 IHC assay comprising the VENTANA SP142 anti-PD-L1 diagnostic antibody; and a detectable expression level of PD-L1 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising a PD-1 axis binding antagonist; and (b) administering the treatment regimen comprising the anti-PD-L1 antibody to the patient identified in step (a) as one who may benefit from the treatment regimen comprising the PD-1 axis binding antagonist.

In another example, provided herein is a method of selecting a therapy for treating a bladder cancer (e.g., a locally advanced or metastatic UC) in a patient in need thereof, the method comprising: (a) determining that a tumor sample obtained from the patient has: a detectable expression level of PD-L1 in tumor-infiltrating immune cells using a PD-L1 IHC assay comprising the VENTANA SP142 anti-PD-L1 diagnostic antibody; and a detectable expression level of PD-L1 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising a PD-1 axis binding antagonist (e.g., an anti-PD-L1 antibody (e.g., atezolizumab)); and (b) selecting a treatment regimen comprising the PD-1 axis binding antagonist for the patient identified in step (a) as one who may benefit from the treatment regimen comprising the PD-1 axis binding antagonist.

In another example, provided herein is a method of identifying a patient having a bladder cancer (e.g., a locally advanced or metastatic UC) who may benefit from a treatment regimen comprising a PD-1 axis binding antagonist (e.g., an anti-PD-L1 antibody (e.g., atezolizumab)), the method comprising: determining that a tumor sample obtained from the patient has: a detectable expression level of PD-L1 in tumor-infiltrating immune cells using a PD-L1 IHC assay comprising the VENTANA SP142 anti-PD-L1 diagnostic antibody; and a detectable expression level of PD-L1 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising a PD-1 axis binding antagonist.

In some examples, the patient is previously untreated for the bladder cancer.

Any suitable reference expression level or cutoffs for the presence or expression level of PD-L1 may be utilized, e.g., any of the reference expression levels or cutoffs described below in Section IV.

In some examples, the patient has a detectable expression level of PD-L1 in tumor-infiltrating immune cells that comprise 5% or more of the tumor sample using a PD-L1 IHC assay comprising the VENTANA SP142 anti-PD-L1 diagnostic antibody.

In some examples, the patient has a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody.

In some examples, the patient has a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody.

In some examples, the presence and/or expression level of PD-L1 in the tumor sample identifies the patient as one who may benefit from the treatment regimen comprising the PD-1 axis binding antagonist.

In one example, provided herein is a method of treating a bladder cancer (e.g., a locally advanced or metastatic UC) in a patient in need thereof, the method comprising administering to the patient a treatment regimen comprising a PD-1 axis binding antagonist (e.g., an anti-PD-L1 antibody (e.g., atezolizumab)), wherein a tumor sample obtained from the patient has been determined to have: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from the treatment regimen comprising the PD-1 axis binding antagonist.

In another example, provided herein is a method of treating a bladder cancer (e.g., a locally advanced or metastatic UC) in a patient in need thereof, the method comprising: (a) determining that a tumor sample obtained from the patient has: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising a PD-1 axis binding antagonist (e.g., an anti-PD-L1 antibody (e.g., atezolizumab)); and (b) administering the treatment regimen comprising the PD-1 axis binding antagonist to the patient identified in step (a) as one who may benefit from the treatment regimen comprising the PD-1 axis binding antagonist.

In another example, provided herein is a PD-1 axis binding antagonist (e.g., an anti-PD-L1 antibody (e.g., atezolizumab)) for use in treatment of a bladder cancer (e.g., a locally advanced or metastatic UC) in a patient in need thereof, the treatment comprising administration to the patient of a treatment regimen comprising a PD-1 axis binding antagonist, wherein a tumor sample obtained from the patient has been determined to have: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from the treatment regimen comprising the PD-1 axis binding antagonist.

In another example, provided herein is a PD-1 axis binding antagonist (e.g., an anti-PD-L1 antibody (e.g., atezolizumab)) for use in a method of treating a bladder cancer (e.g., a locally advanced or metastatic UC) in a patient in need thereof, the method comprising: (a) determining that a tumor sample obtained from the patient has: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising a PD-1 axis binding antagonist; and (b) administering the treatment regimen comprising the anti-PD-L1 antibody to the patient identified in step (a) as one who may benefit from the treatment regimen comprising the PD-1 axis binding antagonist.

In another example, provided herein is a method of selecting a therapy for treating a bladder cancer (e.g., a locally advanced or metastatic UC) in a patient in need thereof, the method comprising: (a) determining that a tumor sample obtained from the patient has: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising a PD-1 axis binding antagonist (e.g., an anti-PD-L1 antibody (e.g., atezolizumab)); and (b) selecting a treatment regimen comprising the PD-1 axis binding antagonist for the patient identified in step (a) as one who may benefit from the treatment regimen comprising the PD-1 axis binding antagonist.

In another example, provided herein is a method of identifying a patient having a bladder cancer (e.g., a locally advanced or metastatic UC) who may benefit from a treatment regimen comprising a PD-1 axis binding antagonist (e.g., an anti-PD-L1 antibody (e.g., atezolizumab)), the method comprising: determining that a tumor sample obtained from the patient has: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising a PD-1 axis binding antagonist.

In some examples, the patient is previously untreated for the bladder cancer.

In one example, provided herein is a method of treating a bladder cancer (e.g., a locally advanced or metastatic UC) in a patient in need thereof, wherein the patient is previously untreated for the bladder cancer, the method comprising administering to the patient a treatment regimen comprising a PD-1 axis binding antagonist (e.g., an anti-PD-L1 antibody (e.g., atezolizumab)), wherein a tumor sample obtained from the patient has been determined to have: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from the treatment regimen comprising the PD-1 axis binding antagonist.

In another example, provided herein is a method of treating a bladder cancer (e.g., a locally advanced or metastatic UC) in a patient in need thereof, wherein the patient is previously untreated for the bladder cancer, the method comprising: (a) determining that a tumor sample obtained from the patient has: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising a PD-1 axis binding antagonist (e.g., an anti-PD-L1 antibody (e.g., atezolizumab)); and (b) administering the treatment regimen comprising the PD-1 axis binding antagonist to the patient identified in step (a) as one who may benefit from the treatment regimen comprising the PD-1 axis binding antagonist.

In another example, provided herein is a PD-1 axis binding antagonist (e.g., an anti-PD-L1 antibody (e.g., atezolizumab)) for use in treatment of a bladder cancer (e.g., a locally advanced or metastatic UC) in a patient in need thereof, wherein the patient is previously untreated for the bladder cancer, the treatment comprising administration to the patient of a treatment regimen comprising a PD-1 axis binding antagonist, wherein a tumor sample obtained from the patient has been determined to have: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from the treatment regimen comprising the PD-1 axis binding antagonist.

In another example, provided herein is a PD-1 axis binding antagonist (e.g., an anti-PD-L1 antibody (e.g., atezolizumab)) for use in a method of treating a bladder cancer (e.g., a locally advanced or metastatic UC) in a patient in need thereof, wherein the patient is previously untreated for the bladder cancer, the method comprising: (a) determining that a tumor sample obtained from the patient has: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising a PD-1 axis binding antagonist; and (b) administering the treatment regimen comprising the anti-PD-L1 antibody to the patient identified in step (a) as one who may benefit from the treatment regimen comprising the PD-1 axis binding antagonist.

In another example, provided herein is a method of selecting a therapy for treating a bladder cancer (e.g., a locally advanced or metastatic UC) in a patient in need thereof, wherein the patient is previously untreated for the bladder cancer, the method comprising: (a) determining that a tumor sample obtained from the patient has: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising a PD-1 axis binding antagonist (e.g., an anti-PD-L1 antibody (e.g., atezolizumab)); and (b) selecting a treatment regimen comprising the PD-1 axis binding antagonist for the patient identified in step (a) as one who may benefit from the treatment regimen comprising the PD-1 axis binding antagonist.

In another example, provided herein is a method of identifying a patient having a bladder cancer (e.g., a locally advanced or metastatic UC) who may benefit from a treatment regimen comprising a PD-1 axis binding antagonist (e.g., an anti-PD-L1 antibody (e.g., atezolizumab)), wherein the patient is previously untreated for the bladder cancer, the method comprising: determining that a tumor sample obtained from the patient has: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising a PD-1 axis binding antagonist.

In some examples, the method further comprises administering the treatment regimen comprising the PD-1 axis binding antagonist (e.g., an anti-PD-L1 antibody (e.g., atezolizumab)) to the patient.

Any suitable PD-1 axis binding antagonist may be used. In some embodiments, the PD-1 axis binding antagonist is described in Section VI below. Other PD-1 axis binding antagonists are known in the art. In some embodiments, the PD-1 axis binding antagonist is selected from the group consisting of a PD-L1 binding antagonist, a PD-1 binding antagonist, and a PD-L2 binding antagonist.

For example, in some examples, the PD-1 axis binding antagonist is an anti-PD-L1 antibody comprising the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In one example, provided herein is a method of treating a locally advanced or metastatic UC in a patient in need thereof, wherein the patient is previously untreated for the locally advanced or metastatic UC, the method comprising administering to the patient a treatment regimen comprising an anti-PD-L1 antibody comprising the following hypervariable regions (HVRs): (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8), wherein a tumor sample obtained from the patient has been determined to have: a detectable expression level of PD-L1 in tumor-infiltrating immune cells that comprise 5% or more of the tumor sample using a PD-L1 immunohistochemical (IHC) assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from the treatment regimen comprising the anti-PD-L1 antibody.

In another example, provided herein is a method of treating a locally advanced or metastatic UC in a patient in need thereof, wherein the patient is previously untreated for the locally advanced or metastatic UC, the method comprising: (a) determining that a tumor sample obtained from the patient has: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising an anti-PD-L1 antibody comprising the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8); and (b) administering the treatment regimen comprising the anti-PD-L1 antibody to the patient identified in step (a) as one who may benefit from the treatment regimen comprising the anti-PD-L1 antibody.

In another example, provided herein is a method of selecting a therapy for treating a locally advanced or metastatic UC in a patient in need thereof, wherein the patient is previously untreated for the locally advanced or metastatic UC, the method comprising: (a) determining that a tumor sample obtained from the patient has: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising an anti-PD-L1 antibody comprising the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8); and (b) selecting a treatment regimen comprising the anti-PD-L1 antibody for the patient identified in step (a) as one who may benefit from the treatment regimen comprising the anti-PD-L1 antibody.

In another example, provided herein is a method of identifying a patient having a locally advanced or metastatic UC who may benefit from a treatment regimen comprising an anti-PD-L1 antibody, wherein the patient is previously untreated for the locally advanced or metastatic UC, the method comprising: determining that a tumor sample obtained from the patient has: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising an anti-PD-L1 antibody, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some examples, the method further comprises administering the treatment regimen comprising the anti-PD-L1 antibody to the patient.

In some examples, the anti-PD-L1 antibody is atezolizumab.

In one example, provided herein is a method of treating a locally advanced or metastatic UC in a patient in need thereof, wherein the patient is previously untreated for the locally advanced or metastatic UC, the method comprising administering to the patient a treatment regimen comprising atezolizumab, wherein a tumor sample obtained from the patient has been determined to have: a detectable expression level of PD-L1 in tumor-infiltrating immune cells that comprise 5% or more of the tumor sample using a PD-L1 immunohistochemical (IHC) assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from the treatment regimen comprising atezolizumab.

In another example, provided herein is a method of treating a locally advanced or metastatic UC in a patient in need thereof, wherein the patient is previously untreated for the locally advanced or metastatic UC, the method comprising: (a) determining that a tumor sample obtained from the patient has: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising atezolizumab; and (b) administering the treatment regimen comprising atezolizumab to the patient identified in step (a) as one who may benefit from the treatment regimen comprising the atezolizumab.

In another example, provided herein is a method of selecting a therapy for treating a locally advanced or metastatic UC in a patient in need thereof, wherein the patient is previously untreated for the locally advanced or metastatic UC, the method comprising: (a) determining that a tumor sample obtained from the patient has: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising atezolizumab; and (b) selecting a treatment regimen comprising the anti-PD-L1 antibody for the patient identified in step (a) as one who may benefit from the treatment regimen comprising atezolizumab.

In another example, provided herein is a method of identifying a patient having a locally advanced or metastatic UC who may benefit from a treatment regimen comprising atezolizumab, wherein the patient is previously untreated for the locally advanced or metastatic UC, the method comprising: determining that a tumor sample obtained from the patient has: a detectable expression level of PD-L1 in tumor-infiltrating immune cells that comprise 5% or more of the tumor sample using a PD-L1 IHC assay comprising the VENTANA SP142 anti-PD-L1 diagnostic antibody; and a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising atezolizumab.

In some examples, the method further comprises administering the treatment regimen comprising the atezolizumab to the patient.

The benefit from the treatment regimen comprising the PD-1 axis binding antagonist (e.g., the anti-PD-L1 antibody (e.g., atezolizumab)) may be, e.g., in terms of overall survival (OS), progression-free survival (PFS), objective response rate (ORR), complete response (CR) rate, and/or duration of response (DOR). The benefit from the treatment regimen comprising the PD-1 axis binding antagonist (e.g., the anti-PD-L1 antibody (e.g., atezolizumab)) may be compared to a suitable reference treatment, e.g., treatment with a platinum-based chemotherapy without the anti-PD-L1 antibody

For example, in some embodiments, the treatment regimen increases the subject's likelihood of having an objective response (e.g., a CR), extends the subject's PFS, extends the subject's OS, and/or extends the subject's DOR as compared to treatment with a platinum-based chemotherapy without the PD-1 axis binding antagonist (e.g., the anti-PD-L1 antibody (e.g., atezolizumab)). In some embodiments, the treatment regimen increases the subject's likelihood of having an objective response as compared to treatment with a platinum-based chemotherapy without the PD-1 axis binding antagonist. In some embodiments, the treatment regimen increases the subject's likelihood of having a CR as compared to treatment with a platinum-based chemotherapy without the PD-1 axis binding antagonist. In some embodiments, the treatment regimen extends the subject's PFS as compared to treatment with a platinum-based chemotherapy without the PD-1 axis binding antagonist. In some embodiments, the treatment regimen extends the subject's OS as compared to treatment with a platinum-based chemotherapy without the PD-1 axis binding antagonist. In some embodiments, the treatment regimen extends the subject's DOR as compared to treatment with a platinum-based chemotherapy without the PD-1 axis binding antagonist.

In some examples, the benefit from the treatment regimen comprising the PD-1 axis binding antagonist (e.g., the anti-PD-L1 antibody (e.g., atezolizumab)) is in terms of OS.

In some examples, the treatment regimen extends the patient's OS by from about 1 months to about 35 months (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months) as compared to treatment with a platinum-based chemotherapy without the PD-1 axis binding antagonist (e.g., the anti-PD-L1 antibody (e.g., atezolizumab)). In some examples, the treatment regimen extends the patient's OS by from about 5.7 months to about 17 months as compared to treatment with a platinum-based chemotherapy without the PD-1 axis binding antagonist (e.g., the anti-PD-L1 antibody (e.g., atezolizumab)). In some particular examples, the treatment regimen extends the patient's OS by about 11.3 months as compared to treatment with a platinum-based chemotherapy without the PD-1 axis binding antagonist (e.g., the anti-PD-L1 antibody (e.g., atezolizumab)).

In some examples, the platinum-based chemotherapy comprises a platinum-based chemotherapeutic agent and a nucleoside analog.

In some examples, the platinum-based chemotherapeutic agent is cisplatin, carboplatin, or oxaliplatin.

In some examples, the platinum-based chemotherapeutic agent is cisplatin.

In some examples, the platinum-based chemotherapeutic agent is carboplatin.

In some examples, the nucleoside analog is gemcitabine.

In some examples, the platinum-based chemotherapy comprises cisplatin and gemcitabine or carboplatin and gemcitabine.

In some examples, the platinum-based chemotherapy comprises cisplatin and gemcitabine.

In some examples, the platinum-based chemotherapy comprises carboplatin and gemcitabine.

In some examples, the anti-PD-L1 antibody comprises: (a) a VH domain comprising the amino acid sequence of SEQ ID NO: 9; and (b) a VL domain comprising the amino acid sequence of SEQ ID NO: 10.

In some examples, the anti-PD-L1 antibody is atezolizumab.

In some embodiments, the PD-1 axis binding antagonist (e.g., an anti-PD-L1 antibody (e.g., atezolizumab)) is administered as a monotherapy.

In other embodiments, the PD-1 axis binding antagonist (e.g., an anti-PD-L1 antibody (e.g., atezolizumab)) is administered in combination with an effective amount of one or more additional therapeutic agents. In some embodiments, the one or more additional therapeutic agents are selected from an anti-neoplastic agent, a chemotherapeutic agent, a growth inhibitory agent, an anti-angiogenic agent, a radiation therapy, or a cytotoxic agent. In some embodiments, the one or more additional therapeutic agents are a platinum-based chemotherapy. In some embodiments, the treatment without the PD-1 axis binding antagonist comprises treatment with a platinum-based chemotherapy.

Any suitable platinum-based chemotherapy may be used, including any platinum-based chemotherapy known in the art or described herein (e.g., in Section VII below). In some embodiments, the platinum-based chemotherapy comprises a platinum-based chemotherapeutic agent and a nucleoside analog. In some embodiments, the platinum-based chemotherapeutic agent is cisplatin, carboplatin, or oxaliplatin.

For example, in some embodiments, the platinum-based chemotherapeutic agent is cisplatin. Any suitable dosing regimen for cisplatin known in the art may be used. In some embodiments, cisplatin is administered to the subject in a 21-day dosing cycle. In some embodiments, cisplatin is administered to the subject intravenously at a dose of about 35 mg/m² to about 140 mg/m². In some embodiments, cisplatin is administered to the subject intravenously at a dose of about 70 mg/m². In some embodiments, cisplatin is administered to the subject intravenously at a dose of about 70 mg/m² on Day-2 to Day 4 of each 21-day dosing cycle. In some embodiments, cisplatin is administered to the subject intravenously at a dose of about 70 mg/m² on Day 1 of each 21-day dosing cycle.

In another example, in other embodiments, the platinum-based chemotherapeutic agent is carboplatin. Any suitable dosing regimen for carboplatin known in the art may be used. In some embodiments, carboplatin is administered to the subject in a 21-day dosing cycle. In some embodiments, carboplatin is administered to the subject intravenously at an area under the curve (AUC) of about 2 to about 9. In some embodiments, carboplatin is administered to the subject intravenously at an area under the curve (AUC) of about 4.5. In some embodiments, carboplatin is administered to the subject intravenously at an area under the curve (AUC) of about 4.5 on Day-2 to Day 4 of each 21-day dosing cycle. In some embodiments, carboplatin is administered to the subject intravenously at an AUC of about 4.5 on Day 1 of each 21-day dosing cycle.

In any of the preceding examples, the platinum-based chemotherapy may include a nucleoside analog. Any suitable nucleoside analog may be used, including any nucleoside analog known in the art or described herein (e.g., in Section VII below). Any suitable dosing regimen for gemcitabine known in the art may be used. In some embodiments, the nucleoside analog is gemcitabine. In some embodiments, gemcitabine is administered to the subject in a 21-day dosing cycle. In some embodiments, gemcitabine is administered to the subject intravenously at a dose of about 500 mg/m² to about 2000 mg/m². In some embodiments, gemcitabine is administered to the subject intravenously at a dose of about 1000 mg/m². In some embodiments, gemcitabine is administered to the subject intravenously at a dose of about 1000 mg/m² on Day-2 to Day 4 and on Day 7 to Day 11 of each 21-day dosing cycle. In some embodiments, gemcitabine is administered to the subject intravenously at a dose of about 1000 mg/m² on Day 1 and Day 8 of each 21-day dosing cycle.

In any of the preceding examples, the platinum-based chemotherapy may include cisplatin and gemcitabine. In other examples, the platinum-based chemotherapy may include carboplatin and gemcitabine.

In some examples, the patient has not received prior chemotherapy for the locally advanced or metastatic UC.

In some examples, the patient has previously received an adjuvant or neoadjuvant chemotherapy or chemoradiation for urothelial carcinoma, and has had a treatment-free interval of more than 12 months between the last administration of the adjuvant or neoadjuvant chemotherapy or chemoradiation and the date of recurrence.

In some examples, the locally advanced or metastatic UC is histologically documented, locally advanced (T4b, any N; or any T, N2-3) or metastatic urothelial carcinoma (mUC) (M1, Stage IV).

In some examples, the UC is locally advanced UC.

In some examples, the locally advanced UC is inoperable.

In some examples, the UC is metastatic UC.

In some examples, the patient may be eligible for any suitable platinum-based chemotherapy. Eligibility for a platinum-based chemotherapy may be as described herein or according to criteria known in the art. For example, criteria for defining patients who are cisplatin-eligible or cisplatin-ineligible are known in the art, e.g., as described in Galsky et al. Lancet. Oncol. 12:211-4, 2011, which is incorporated herein by reference in its entirety. In some embodiments, the subject is eligible for treatment with a platinum-based chemotherapy comprising cisplatin. In some embodiments, the subject is eligible for treatment with a platinum-based chemotherapy comprising carboplatin.

In other examples, the patient may be ineligible for a platinum-based chemotherapy. In some embodiments, the subject is ineligible for treatment with a platinum-based chemotherapy comprising cisplatin. In some embodiments, the subject is ineligible for treatment with a platinum-based chemotherapy comprising carboplatin.

In some examples, the patient is a human.

In some examples, the tumor sample obtained from the patient has the presence of discernible PD-L1 staining of any intensity in tumor-infiltrating immune cells covering ≥5% of tumor area occupied by tumor cells, associated intratumoral, and contiguous peritumoral stroma, as determined by the PD-L1 IHC assay comprising the VENTANA SP142 anti-PD-L1 diagnostic antibody.

In some examples, the tumor sample obtained from the patient has a CPS of ≥10 using a PD-L1 IHC assay comprising the Dako 22C3 anti-PD-L1 diagnostic antibody.

In some examples, the tumor sample obtained from the patient has a CPS of ≥10 using a PD-L1 IHC assay comprising the VENTANA SP263 anti-PD-L1 diagnostic antibody.

In some examples, the tumor sample obtained from the patient has a CPS of ≥10 using a PD-L1 IHC assay comprising the 28-8 anti-PD-L1 diagnostic antibody.

In any of the preceding examples, the PD-1 axis binding antagonist (e.g., the anti-PD-L1 antibody, e.g., atezolizumab) may be administered in one or more dosing cycles.

In any of the preceding examples, each dosing cycle may have any suitable length, e.g., about 7 days, about 14 days, about 21 days, about 28 days, or longer. In some embodiments, each dosing cycle is about 21 days.

Any suitable number of dosing cycles may be used, e.g., 1, 2, 3, 4, 5, 6, 7, 8, 9, 10, 11, 12, 13, 14, 15, 20, 25, 30, 35, 40, 45, 50, or more dosing cycles. In some embodiments, 10 or fewer dosing cycles may be used. In some embodiments, 20 or fewer dosing cycles are used. In some embodiments, 25 or fewer dosing cycles are used.

In some embodiments, the tumor sample is a formalin-fixed and paraffin-embedded (FFPE) tumor sample, an archival tumor sample, a fresh tumor sample, or a frozen tumor sample. The presence and/or expression level of any of the biomarkers described herein (e.g., PD-L1) can be determined using any method described herein (e.g., in Section IV or in Example 2 below), or using approaches that are known in the art.

As a general proposition, the therapeutically effective amount of a PD-1 axis binding antagonist (e.g., an anti-PD-L1 antibody (e.g., atezolizumab)) administered to a human will be in the range of about 0.01 to about 50 mg/kg of patient body weight, whether by one or more administrations. In some embodiments, for example, the antagonist (e.g., a PD-1 axis binding antagonist (e.g., an anti-PD-L1 antibody (e.g., atezolizumab))) is administered in a dose of about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, weekly, every two weeks, every three weeks, or every four weeks, for example. In some embodiments, the antagonist (e.g., a PD-1 axis binding antagonist (e.g., an anti-PD-L1 antibody (e.g., atezolizumab)) is administered at 15 mg/kg. However, other dosage regimens may be useful. In one embodiment, a PD-1 axis binding antagonist (e.g., an anti-PD-L1 antibody (e.g., atezolizumab)) is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, or about 1800 mg. In some embodiments, the antagonist (e.g., an anti-PD-L1 antibody (e.g., atezolizumab)) may be administered at a dose of about 1000 mg to about 1400 mg every three weeks (e.g., about 1100 mg to about 1300 mg every three weeks, e.g., about 1150 mg to about 1250 mg every three weeks). In some embodiments, the antagonist (e.g., an anti-PD-L1 antibody (e.g., atezolizumab)) is administered to the subject intravenously at a dose of about 840 mg every 2 weeks, about 1200 mg every 3 weeks, or about 1680 mg every 4 weeks. In some embodiments, the antagonist (e.g., an anti-PD-L1 antibody (e.g., atezolizumab)) is administered at a dose of about 1200 mg of atezolizumab every three weeks. The dose may be administered as a single dose or as multiple doses (e.g., 2 or 3 doses), such as infusions. The dose of the antibody administered in a combination treatment may be reduced as compared to a single treatment. In some embodiments, the treatment regimen comprises administering intravenously to the subject about 1200 mg of atezolizumab every three weeks. The progress of this therapy is easily monitored by conventional techniques.

In some instances, a patient is administered a total of 1 to 50 doses of a PD-1 axis binding antagonist (e.g., an anti-PD-L1 antibody (e.g., atezolizumab)), e.g., 1 to 50 doses, 1 to 45 doses, 1 to 40 doses, 1 to 35 doses, 1 to 30 doses, 1 to 25 doses, 1 to 20 doses, 1 to 15 doses, 1 to 10 doses, 1 to 5 doses, 2 to 50 doses, 2 to 45 doses, 2 to 40 doses, 2 to 35 doses, 2 to 30 doses, 2 to 25 doses, 2 to 20 doses, 2 to 15 doses, 2 to 10 doses, 2 to 5 doses, 3 to 50 doses, 3 to 45 doses, 3 to 40 doses, 3 to 35 doses, 3 to 30 doses, 3 to 25 doses, 3 to 20 doses, 3 to 15 doses, 3 to 10 doses, 3 to 5 doses, 4 to 50 doses, 4 to 45 doses, 4 to 40 doses, 4 to 35 doses, 4 to 30 doses, 4 to 25 doses, 4 to 20 doses, 4 to 15 doses, 4 to 10 doses, 4 to 5 doses, 5 to 50 doses, 5 to 45 doses, 5 to 40 doses, 5 to 35 doses, 5 to 30 doses, 5 to 25 doses, 5 to 20 doses, 5 to 15 doses, 5 to 10 doses, 10 to 50 doses, 10 to 45 doses, 10 to 40 doses, 10 to 35 doses, 10 to 30 doses, 10 to 25 doses, 10 to 20 doses, 10 to 15 doses, 15 to 50 doses, 15 to 45 doses, 15 to 40 doses, 15 to 35 doses, 15 to 30 doses, 15 to 25 doses, 15 to 20 doses, 20 to 50 doses, 20 to 45 doses, 20 to 40 doses, 20 to 35 doses, 20 to 30 doses, 20 to 25 doses, 25 to 50 doses, 25 to 45 doses, 25 to 40 doses, 25 to 35 doses, 25 to 30 doses, 30 to 50 doses, 30 to 45 doses, 30 to 40 doses, 30 to 35 doses, 35 to 50 doses, 35 to 45 doses, 35 to 40 doses, 40 to 50 doses, 40 to 45 doses, or 45 to 50 doses. In particular instances, the doses may be administered intravenously.

Atezolizumab may be administered to the subject at any suitable dosage. In some embodiments, atezolizumab is administered to the subject intravenously at a dose of about 840 mg every 2 weeks, about 1200 mg every 3 weeks, or about 1680 mg every 4 weeks. In some embodiments, atezolizumab is administered to the subject intravenously at a dose of about 1200 mg every 3 weeks. In some embodiments, atezolizumab is administered to the subject in a 21-day dosing cycle. In some embodiments, atezolizumab is administered to the subject intravenously at a dose of about 1200 mg on Day-2 to Day 4 of each 21-day dosing cycle. In some embodiments, atezolizumab is administered to the subject intravenously at a dose of about 1200 mg on Day 1 of each 21-day dosing cycle.

In a preferred embodiment, the PD-1 axis binding antagonist (e.g., an anti-PD-L1 antibody (e.g., atezolizumab)) is administered intravenously. In one example, atezolizumab may be administered intravenously over 60 minutes; if the first infusion is tolerated, all subsequent infusions may be delivered over 30 minutes. In some examples, the PD-1 axis binding antagonist (e.g., an anti-PD-L1 antibody (e.g., atezolizumab)) is not administered as an intravenous push or bolus.

In some embodiments, the PD-1 axis binding antagonist (e.g., an anti-PD-L1 antibody (e.g., atezolizumab)) and one or more additional therapeutic agents (e.g., a platinum-based chemotherapy (e.g., cisplatin or carboplatin and gemcitabine)) are administered in a single dosing regimen. The administration of these agents may be concurrent or separate within the context of the dosing regimen.

The PD-1 axis binding antagonist (e.g., an anti-PD-L1 antibody (e.g., atezolizumab)) and/or one or more additional therapeutic agents (e.g., a platinum-based chemotherapy (e.g., cisplatin or carboplatin and gemcitabine)) may be administered in any suitable manner known in the art. For example, the PD-1 axis binding antagonist (e.g., an anti-PD-L1 antibody (e.g., atezolizumab)) and one or more additional therapeutic agents (e.g., a platinum-based chemotherapy (e.g., cisplatin or carboplatin and gemcitabine)) may be administered sequentially (at different times) or concurrently (at the same time). In some embodiments, the PD-1 axis binding antagonist is administered prior to the one or more additional therapeutic agents (e.g., the platinum-based chemotherapy (e.g., cisplatin or carboplatin and gemcitabine)). In other embodiments, the PD-1 axis binding antagonist is administered after the one or more additional therapeutic agents (e.g., the platinum-based chemotherapy (e.g., cisplatin or carboplatin and gemcitabine)). In yet other embodiments, the PD-1 axis binding antagonist is administered concurrently with the one or more additional therapeutic agents (e.g., the platinum-based chemotherapy (e.g., cisplatin or carboplatin and gemcitabine)). In some embodiments, the PD-1 axis binding antagonist (e.g., an anti-PD-L1 antibody (e.g., atezolizumab)) is in a separate composition as the one or more additional therapeutic agents (e.g., the platinum-based chemotherapy (e.g., cisplatin or carboplatin and gemcitabine)). In some embodiments, the PD-1 axis binding antagonist (e.g., an anti-PD-L1 antibody (e.g., atezolizumab)) is in the same composition as the one or more additional therapeutic agents (e.g., the platinum-based chemotherapy (e.g., cisplatin or carboplatin and gemcitabine)).

The PD-1 axis binding antagonist (e.g., an anti-PD-L1 antibody (e.g., atezolizumab)) and/or one or more additional therapeutic agents (e.g., a platinum-based chemotherapy (e.g., cisplatin or carboplatin and gemcitabine)) may be administered by the same route of administration or by different routes of administration. In some embodiments, the PD-1 axis binding antagonist (e.g., an anti-PD-L1 antibody (e.g., atezolizumab)) is administered intravenously, intramuscularly, subcutaneously, topically, orally, transdermally, intraperitoneally, intraorbitally, by implantation, by inhalation, intrathecally, intraventricularly, or intranasally. In some embodiments, the platinum-based chemotherapy (e.g., cisplatin or carboplatin and gemcitabine) is administered intravenously, intramuscularly, subcutaneously, topically, orally, transdermally, intraperitoneally, intraorbitally, by implantation, by inhalation, intrathecally, intraventricularly, or intranasally. An effective amount of the PD-1 axis binding antagonist (e.g., an anti-PD-L1 antibody (e.g., atezolizumab)) and/or one or more additional therapeutic agents (e.g., a platinum-based chemotherapy (e.g., cisplatin or carboplatin and gemcitabine)) may be administered for prevention or treatment of disease. The appropriate dosage of the PD-1 axis binding antagonist (e.g., an anti-PD-L1 antibody (e.g., atezolizumab)) and/or the one or more additional therapeutic agents (e.g., a platinum-based chemotherapy (e.g., cisplatin or carboplatin and gemcitabine)) may be determined based on the type of disease to be treated, the type of the PD-1 axis binding antagonist, the severity and course of the disease, the clinical condition of the individual, the individual's clinical history and response to the treatment, and the discretion of the attending physician. In some embodiments, the PD-1 axis binding antagonist (e.g., an anti-PD-L1 antibody (e.g., atezolizumab)) and/or the platinum-based chemotherapy (e.g., cisplatin or carboplatin and gemcitabine) is administered intravenously by infusion.

For example, when administering with chemotherapy with or without bevacizumab, atezolizumab may be administered at a dose of 1200 mg every 3 weeks prior to chemotherapy and bevacizumab. In another example, following completion of 4-6 cycles of chemotherapy, and if bevacizumab is discontinued, atezolizumab may be administered at a dose of 840 mg every 2 weeks, 1200 mg every 3 weeks, or 1680 mg every four weeks. In another example, atezolizumab may be administered at a dose of 840 mg, followed by 100 mg/m² of paclitaxel protein-bound (e.g., nab-paclitaxel); for each 28-day cycle, atezolizumab is administered on days 1 and 15, and paclitaxel protein-bound is administered on days 1, 8, and 15. In another example, when administering with carboplatin and etoposide, atezolizumab can be administered at a dose of 1200 mg every 3 weeks prior to chemotherapy. In yet another example, following completion of 4 cycles of carboplatin and etoposide, atezolizumab may be administered at a dose of 840 mg every 2 weeks, 1200 mg every 3 weeks, or 1680 mg every 4 weeks. In another example, following completion of a 28-day cycle of cobimenitib and vemurafenib, atezolizumab may be administered at a dose of 840 mg every 2 weeks with cobimetinib at a dose of 60 mg orally once daily (21 days on, 7 days off) and vemurafenib at a dose of 720 mg orally twice daily.

In some embodiments, the treatment may further comprise an additional therapy. Any suitable additional therapy known in the art or described herein may be used. The additional therapy may be radiation therapy, surgery (e.g., transurethral bladder tumor resection (TURBT) or cystectomy (including a partial or radical cystectomy)), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, and the like). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PI3K/AKT/mTOR pathway, HSP90 inhibitor, tubulin inhibitor, apoptosis inhibitor, and/or chemopreventative agent. The additional therapy may be one or more of the chemotherapeutic agents described herein.

In some instances, the additional therapy is the administration of side-effect limiting agents (e.g., agents intended to lessen the occurrence and/or severity of side effects of treatment, such as anti-nausea agents, a corticosteroid (e.g., prednisone or an equivalent, e.g., at a dose of 1-2 mg/kg/day), hormone replacement medicine(s), and the like).

### III. Combination Therapies

Also provided herein are methods for treating or delaying progression of bladder cancer (e.g., UC, including locally advanced or metastatic UC) in a subject comprising administering to the subject a treatment regimen comprising an effective amount of a PD-1 axis binding antagonist (e.g., an anti-PD-L1 antibody (e.g., atezolizumab)) in conjunction with another anti-cancer agent or cancer therapy. For example provided herein are methods for treating or delaying progression of bladder cancer (e.g., UC, including locally advanced or metastatic UC) in a subject comprising administering to the subject a treatment regimen comprising an effective amount of a PD-1 axis binding antagonist (e.g., an anti-PD-L1 antibody (e.g., atezolizumab)) and a platinum-based chemotherapy (e.g., cisplatin or carboplatin and gemcitabine) in conjunction with another anti-cancer agent or cancer therapy. In some embodiments, the methods comprise administering to the individual a PD-1 axis binding antagonist (e.g., an anti-PD-L1 antibody (e.g., atezolizumab)), a platinum-based chemotherapy (e.g., cisplatin or carboplatin and gemcitabine), and an additional therapeutic agent. Any of the combinations described below may be used, e.g., in a method as described in Section II above.

In some embodiments, a PD-1 axis binding antagonist (e.g., an anti-PD-L1 antibody (e.g., atezolizumab)) and/or a platinum-based chemotherapy (e.g., cisplatin or carboplatin and gemcitabine) may be administered in conjunction with an additional chemotherapy or chemotherapeutic agent. In some embodiments, a PD-1 axis binding antagonist (e.g., an anti-PD-L1 antibody (e.g., atezolizumab)) and/or a platinum-based chemotherapy (e.g., cisplatin or carboplatin and gemcitabine) may be administered in conjunction with a radiation therapy or radiotherapeutic agent. In some embodiments, a PD-1 axis binding antagonist (e.g., an anti-PD-L1 antibody (e.g., atezolizumab)) and/or a platinum-based chemotherapy (e.g., cisplatin or carboplatin and gemcitabine) may be administered in conjunction with a targeted therapy or targeted therapeutic agent. In some embodiments, a PD-1 axis binding antagonist (e.g., an anti-PD-L1 antibody (e.g., atezolizumab)) and/or a platinum-based chemotherapy (e.g., cisplatin or carboplatin and gemcitabine) may be administered in conjunction with an immunotherapy or immunotherapeutic agent, for example, a monoclonal antibody.

Without wishing to be bound to theory, it is thought that enhancing T cell stimulation, by promoting an activating co-stimulatory molecule or by inhibiting a negative co-stimulatory molecule, may promote tumor cell death, thereby treating or delaying progression of cancer. In some embodiments, a PD-1 axis binding antagonist (e.g., an anti-PD-L1 antibody (e.g., atezolizumab)) and/or a platinum-based chemotherapy (e.g., cisplatin or carboplatin and gemcitabine) may be administered in conjunction with an agonist directed against an activating co-stimulatory molecule. In some embodiments, an activating co-stimulatory molecule may include CD40, CD226, CD28, OX40, GITR, CD137, CD27, HVEM, or CD127. In some embodiments, the agonist directed against an activating co-stimulatory molecule is an agonist antibody that binds to CD40, CD226, CD28, OX40, GITR, CD137, CD27, HVEM, or CD127. In some embodiments, a PD-1 axis binding antagonist (e.g., an anti-PD-L1 antibody (e.g., atezolizumab)) and/or a platinum-based chemotherapy (e.g., cisplatin or carboplatin and gemcitabine) may be administered in conjunction with an antagonist directed against an inhibitory co-stimulatory molecule. In some embodiments, an inhibitory co-stimulatory molecule may include CTLA-4 (also known as CD152), PD-1, TIM-3, BTLA, VISTA, LAG-3, B7-H3, B7-H4, IDO, TIGIT, MICA/B, or arginase. In some embodiments, the antagonist directed against an inhibitory co-stimulatory molecule is an antagonist antibody that binds to CTLA-4, PD-1, TIM-3, BTLA, VISTA, LAG-3, B7-H3, B7-H4, IDO, TIGIT, MICA/B, or arginase.

In some embodiments, a PD-1 axis binding antagonist (e.g., an anti-PD-L1 antibody (e.g., atezolizumab)) and/or a platinum-based chemotherapy (e.g., cisplatin or carboplatin and gemcitabine) may be administered in conjunction with an antagonist directed against CTLA-4 (also known as CD152), for example, a blocking antibody. In some embodiments, a PD-1 axis binding antagonist (e.g., an anti-PD-L1 antibody (e.g., atezolizumab)) and/or a platinum-based chemotherapy (e.g., cisplatin or carboplatin and gemcitabine) may be administered in conjunction with ipilimumab (also known as MDX-010, MDX-101, or YERVOY®). In some embodiments, a PD-1 axis binding antagonist (e.g., an anti-PD-L1 antibody (e.g., atezolizumab)) and/or a platinum-based chemotherapy (e.g., cisplatin or carboplatin and gemcitabine) may be administered in conjunction with tremelimumab (also known as ticilimumab or CP-675,206). In some embodiments, a PD-1 axis binding antagonist (e.g., an anti-PD-L1 antibody (e.g., atezolizumab)) and/or a platinum-based chemotherapy (e.g., cisplatin or carboplatin and gemcitabine) may be administered in conjunction with an antagonist directed against B7-H3 (also known as CD276), for example, a blocking antibody. In some embodiments, a PD-1 axis binding antagonist (e.g., an anti-PD-L1 antibody (e.g., atezolizumab)) and/or a platinum-based chemotherapy (e.g., cisplatin or carboplatin and gemcitabine) may be administered in conjunction with MGA271. In some embodiments, a PD-1 axis binding antagonist (e.g., an anti-PD-L1 antibody (e.g., atezolizumab)) and/or a platinum-based chemotherapy (e.g., cisplatin or carboplatin and gemcitabine) may be administered in conjunction with an antagonist directed against a TGF beta, for example, metelimumab (also known as CAT-192), fresolimumab (also known as GC1008), or LY2157299.

In some embodiments, a PD-1 axis binding antagonist (e.g., an anti-PD-L1 antibody (e.g., atezolizumab)) and/or a platinum-based chemotherapy (e.g., cisplatin or carboplatin and gemcitabine) may be administered in conjunction with a treatment comprising adoptive transfer of a T cell (e.g., a cytotoxic T cell or CTL) expressing a chimeric antigen receptor (CAR). In some embodiments, a PD-1 axis binding antagonist (e.g., an anti-PD-L1 antibody (e.g., atezolizumab)) and/or a platinum-based chemotherapy (e.g., cisplatin or carboplatin and gemcitabine) may be administered in conjunction with a treatment comprising adoptive transfer of a T cell comprising a dominant-negative TGF beta receptor, e.g., a dominant-negative TGF beta type II receptor. In some embodiments, a PD-1 axis binding antagonist (e.g., an anti-PD-L1 antibody (e.g., atezolizumab)) and/or a platinum-based chemotherapy (e.g., cisplatin or carboplatin and gemcitabine) may be administered in conjunction with a treatment comprising a HERCREEM protocol (see, e.g., ClinicalTrials.gov Identifier NCT00889954).

In some embodiments, a PD-1 axis binding antagonist and/or a platinum-based chemotherapy may be administered in conjunction with an agonist directed against CD137 (also known as TNFRSF9, 4-1BB, or ILA), for example, an activating antibody. In some embodiments, a PD-1 axis binding antagonist and/or a platinum-based chemotherapy may be administered in conjunction with urelumab (also known as BMS-663513). In some embodiments, a PD-1 axis binding antagonist and/or a platinum-based chemotherapy may be administered in conjunction with an agonist directed against CD40, for example, an activating antibody. In some embodiments, a PD-1 axis binding antagonist and/or a platinum-based chemotherapy may be administered in conjunction with CP-870893. In some embodiments, a PD-1 axis binding antagonist and/or a platinum-based chemotherapy may be administered in conjunction with an agonist directed against OX40 (also known as CD134), for example, an activating antibody. In some embodiments, a PD-1 axis binding antagonist and/or a platinum-based chemotherapy may be administered in conjunction with an anti-OX40 antibody (e.g., AgonOX). In some embodiments, a PD-1 axis binding antagonist and/or a platinum-based chemotherapy may be administered in conjunction with an agonist directed against CD27, for example, an activating antibody. In some embodiments, a PD-1 axis binding antagonist and/or a platinum-based chemotherapy may be administered in conjunction with CDX-1127. In some embodiments, a PD-1 axis binding antagonist and/or a platinum-based chemotherapy may be administered in conjunction with an antagonist directed against indoleamine-2,3-dioxygenase (IDO). In some embodiments, the IDO antagonist is 1-methyl-D-tryptophan (also known as 1-D-MT).

In some embodiments, a PD-1 axis binding antagonist and/or a platinum-based chemotherapy may be administered in conjunction with an antibody-drug conjugate. In some embodiments, the antibody-drug conjugate comprises mertansine or monomethyl auristatin E (MMAE). In some embodiments, a PD-1 axis binding antagonist and/or a platinum-based chemotherapy may be administered in conjunction with and anti-NaPi2b antibody-MMAE conjugate (also known as DNIB0600A or RG7599). In some embodiments, a PD-1 axis binding antagonist and/or a platinum-based chemotherapy may be administered in conjunction with trastuzumab emtansine (also known as T-DM1, ado-trastuzumab emtansine, or KADCYLA®, Genentech). In some embodiments, a PD-1 axis binding antagonist and/or a platinum-based chemotherapy may be administered in conjunction with DMUC5754A. In some embodiments, a PD-1 axis binding antagonist and/or a platinum-based chemotherapy may be administered in conjunction with an antibody-drug conjugate targeting the endothelin B receptor (EDNBR), for example, an antibody directed against EDNBR conjugated with MMAE.

In some embodiments, a PD-1 axis binding antagonist and/or a platinum-based chemotherapy may be administered in conjunction with an angiogenesis inhibitor. In some embodiments, a PD-1 axis binding antagonist and/or a platinum-based chemotherapy may be administered in conjunction with an antibody directed against angiopoietin 2 (also known as Ang2). In some embodiments, a PD-1 axis binding antagonist and/or a platinum-based chemotherapy may be administered in conjunction with MEDI3617.

In some embodiments, a PD-1 axis binding antagonist and/or a platinum-based chemotherapy may be administered in conjunction with an antineoplastic agent. In some embodiments, a PD-1 axis binding antagonist and/or a platinum-based chemotherapy may be administered in conjunction with an agent targeting CSF-1R (also known as M-CSFR or CD115). In some embodiments, a PD-1 axis binding antagonist and/or a platinum-based chemotherapy may be administered in conjunction with anti-CSF-1R (also known as IMC-CS4). In some embodiments, a PD-1 axis binding antagonist and/or a platinum-based chemotherapy may be administered in conjunction with an interferon, for example interferon alpha or interferon gamma. In some embodiments, a PD-1 axis binding antagonist and/or a platinum-based chemotherapy may be administered in conjunction with Roferon-A (also known as recombinant Interferon alpha-2a). In some embodiments, a PD-1 axis binding antagonist and/or a platinum-based chemotherapy may be administered in conjunction with GM-CSF (also known as recombinant human granulocyte macrophage colony stimulating factor, rhu GM-CSF, sargramostim, or LEUKINE®). In some embodiments, a PD-1 axis binding antagonist and/or a platinum-based chemotherapy may be administered in conjunction with IL-2 (also known as aldesleukin or PROLEUKIN®). In some embodiments, a PD-1 axis binding antagonist and/or a platinum-based chemotherapy may be administered in conjunction with IL-12. In some embodiments, a PD-1 axis binding antagonist and/or a platinum-based chemotherapy may be administered in conjunction with an antibody targeting CD20. In some embodiments, the antibody targeting CD20 is obinutuzumab (also known as GA101 or GAZYVAR) or rituximab. In some embodiments, a PD-1 axis binding antagonist and/or a platinum-based chemotherapy may be administered in conjunction with an antibody targeting GITR. In some embodiments, the antibody targeting GITR is TRX518.

In some embodiments, a PD-1 axis binding antagonist and/or a platinum-based chemotherapy may be administered in conjunction with a cancer vaccine. In some embodiments, the cancer vaccine is a peptide cancer vaccine, which in some embodiments is a personalized peptide vaccine. In some embodiments the peptide cancer vaccine is a multivalent long peptide, a multi-peptide, a peptide cocktail, a hybrid peptide, or a peptide-pulsed dendritic cell vaccine (see, e.g., Yamada et al., Cancer Sci, 104:14-21, 2013). In some embodiments, a PD-1 axis binding antagonist and/or a platinum-based chemotherapy may be administered in conjunction with an adjuvant. In some embodiments, a PD-1 axis binding antagonist and/or a platinum-based chemotherapy may be administered in conjunction with a treatment comprising a TLR agonist, for example, Poly-ICLC (also known as HILTONOL®), LPS, MPL, or CpG ODN. In some embodiments, a PD-1 axis binding antagonist and/or a platinum-based chemotherapy may be administered in conjunction with tumor necrosis factor (TNF) alpha. In some embodiments, a PD-1 axis binding antagonist and/or a platinum-based chemotherapy may be administered in conjunction with IL-1. In some embodiments, a PD-1 axis binding antagonist and/or a platinum-based chemotherapy may be administered in conjunction with HMGB1. In some embodiments, a PD-1 axis binding antagonist and/or a platinum-based chemotherapy may be administered in conjunction with an IL-10 antagonist. In some embodiments, a PD-1 axis binding antagonist and/or a platinum-based chemotherapy may be administered in conjunction with an IL-4 antagonist. In some embodiments, a PD-1 axis binding antagonist and/or a platinum-based chemotherapy may be administered in conjunction with an IL-13 antagonist. In some embodiments, a PD-1 axis binding antagonist and/or a platinum-based chemotherapy may be administered in conjunction with an HVEM antagonist. In some embodiments, a PD-1 axis binding antagonist and/or a platinum-based chemotherapy may be administered in conjunction with an ICOS agonist, e.g., by administration of ICOS-L, or an agonistic antibody directed against ICOS. In some embodiments, a PD-1 axis binding antagonist and/or a platinum-based chemotherapy may be administered in conjunction with a treatment targeting CX3CL1. In some embodiments, a PD-1 axis binding antagonist and/or a platinum-based chemotherapy may be administered in conjunction with a treatment targeting CXCL9. In some embodiments, a PD-1 axis binding antagonist and/or a platinum-based chemotherapy may be administered in conjunction with a treatment targeting CXCL10. In some embodiments, a PD-1 axis binding antagonist and/or a platinum-based chemotherapy may be administered in conjunction with a treatment targeting CCL5. In some embodiments, a PD-1 axis binding antagonist and/or a platinum-based chemotherapy may be administered in conjunction with an LFA-1 or ICAM1 agonist. In some embodiments, a PD-1 axis binding antagonist and/or a platinum-based chemotherapy may be administered in conjunction with a Selectin agonist.

In some embodiments, a PD-1 axis binding antagonist and/or a platinum-based chemotherapy may be administered in conjunction with a targeted therapy. In some embodiments, a PD-1 axis binding antagonist and/or a platinum-based chemotherapy may be administered in conjunction with an inhibitor of B-Raf. In some embodiments, a PD-1 axis binding antagonist and/or a platinum-based chemotherapy may be administered in conjunction with vemurafenib (also known as ZELBORAF®). In some embodiments, a PD-1 axis binding antagonist and/or a platinum-based chemotherapy may be administered in conjunction with dabrafenib (also known as TAFINLAR®). In some embodiments, a PD-1 axis binding antagonist and/or a platinum-based chemotherapy may be administered in conjunction with erlotinib (also known as TARCEVA®). In some embodiments, a PD-1 axis binding antagonist and/or a platinum-based chemotherapy may be administered in conjunction with an inhibitor of a MEK, such as MEK1 (also known as MAP2K1) or MEK2 (also known as MAP2K2). In some embodiments, a PD-1 axis binding antagonist and/or a platinum-based chemotherapy may be administered in conjunction with cobimetinib (also known as GDC-0973 or XL-518). In some embodiments, a PD-1 axis binding antagonist and/or a platinum-based chemotherapy may be administered in conjunction with trametinib (also known as MEKINIST®). In some embodiments, a PD-1 axis binding antagonist and/or a platinum-based chemotherapy may be administered in conjunction with an inhibitor of K-Ras. In some embodiments, a PD-1 axis binding antagonist and/or a platinum-based chemotherapy may be administered in conjunction with an inhibitor of c-Met. In some embodiments, a PD-1 axis binding antagonist and/or a platinum-based chemotherapy may be administered in conjunction with onartuzumab (also known as MetMAb). In some embodiments, a PD-1 axis binding antagonist and/or a platinum-based chemotherapy may be administered in conjunction with an inhibitor of Alk. In some embodiments, a PD-1 axis binding antagonist and/or a platinum-based chemotherapy may be administered in conjunction with AF802 (also known as CH5424802 or alectinib). In some embodiments, a PD-1 axis binding antagonist and/or a platinum-based chemotherapy may be administered in conjunction with an inhibitor of a phosphatidylinositol 3-kinase (PI3K). In some embodiments, a PD-1 axis binding antagonist and/or a platinum-based chemotherapy may be administered in conjunction with BKM120. In some embodiments, a PD-1 axis binding antagonist and/or a platinum-based chemotherapy may be administered in conjunction with idelalisib (also known as GS-1101 or CAL-101). In some embodiments, a PD-1 axis binding antagonist and/or a platinum-based chemotherapy may be administered in conjunction with perifosine (also known as KRX-0401). In some embodiments, a PD-1 axis binding antagonist and/or a platinum-based chemotherapy may be administered in conjunction with an inhibitor of an Akt. In some embodiments, a PD-1 axis binding antagonist and/or a platinum-based chemotherapy may be administered in conjunction with MK2206. In some embodiments, a PD-1 axis binding antagonist and/or a platinum-based chemotherapy may be administered in conjunction with GSK690693. In some embodiments, a PD-1 axis binding antagonist and/or a platinum-based chemotherapy may be administered in conjunction with GDC-0941. In some embodiments, a PD-1 axis binding antagonist and/or a platinum-based chemotherapy may be administered in conjunction with an inhibitor of mTOR. In some embodiments, a PD-1 axis binding antagonist and/or a platinum-based chemotherapy may be administered in conjunction with sirolimus (also known as rapamycin). In some embodiments, a PD-1 axis binding antagonist and/or a platinum-based chemotherapy may be administered in conjunction with temsirolimus (also known as CCI-779 or TORISEL®). In some embodiments, a PD-1 axis binding antagonist and/or a platinum-based chemotherapy may be administered in conjunction with everolimus (also known as RAD001). In some embodiments, a PD-1 axis binding antagonist and/or a platinum-based chemotherapy may be administered in conjunction with ridaforolimus (also known as AP-23573, MK-8669, or deforolimus). In some embodiments, a PD-1 axis binding antagonist and/or a platinum-based chemotherapy may be administered in conjunction with OSI-027. In some embodiments, a PD-1 axis binding antagonist and/or a platinum-based chemotherapy may be administered in conjunction with AZD8055. In some embodiments, a PD-1 axis binding antagonist and/or a platinum-based chemotherapy may be administered in conjunction with INK128. In some embodiments, a PD-1 axis binding antagonist and/or a platinum-based chemotherapy may be administered in conjunction with a dual PI3K/mTOR inhibitor. In some embodiments, a PD-1 axis binding antagonist and/or a platinum-based chemotherapy may be administered in conjunction with XL765. In some embodiments, a PD-1 axis binding antagonist and/or a platinum-based chemotherapy may be administered in conjunction with GDC-0980. In some embodiments, a PD-1 axis binding antagonist and/or a platinum-based chemotherapy may be administered in conjunction with BEZ235 (also known as NVP-BEZ235). In some embodiments, a PD-1 axis binding antagonist and/or a platinum-based chemotherapy may be administered in conjunction with BGT226. In some embodiments, a PD-1 axis binding antagonist and/or a platinum-based chemotherapy may be administered in conjunction with GSK2126458. In some embodiments, a PD-1 axis binding antagonist and/or a platinum-based chemotherapy may be administered in conjunction with PF-04691502. In some embodiments, a PD-1 axis binding antagonist and/or a platinum-based chemotherapy may be administered in conjunction with PF-05212384 (also known as PKI-587).

In any of the preceding embodiments, the PD-1 axis binding antagonist may be a human PD-1 axis binding antagonist.

In any of the preceding embodiments, the PD-1 axis binding antagonist is an anti-PD-L1 antibody (e.g., atezolizumab).

In any of the preceding embodiments, the platinum-based chemotherapy includes a platinum-based chemotherapeutic agent (e.g., cisplatin or carboplatin). In some embodiments, the platinum-based chemotherapy includes cisplatin. In some embodiments, the platinum-based chemotherapy includes carboplatin. In some embodiments, the platinum-based chemotherapy further includes one or more additional chemotherapeutic agents, e.g., a nucleoside analog. In some embodiments, the nucleoside analog is gemcitabine. In some embodiments, the platinum-based chemotherapy includes cisplatin and gemcitabine. In other embodiments, the platinum-based chemotherapy includes carboplatin and gemcitabine.

### IV. Assessment of PD-L1 Expression

The presence and/or expression level of PD-L1 may be assessed in a patient identified, selected, stratified, and/or treated according to any of the methods and compositions for use described herein. The methods and compositions for use may include determining the expression level of PD-L1 in a biological sample (e.g., a tumor sample) obtained from the patient. In other examples, the expression level of PD-L1 in a biological sample (e.g., a tumor sample) obtained from the patient has been determined prior to initiation of treatment or after initiation of treatment. PD-L1 expression may be determined using any suitable approach. For example, PD-L1 expression may be determined as described in U.S. patent application Ser. Nos. 15/787,988 and 15/790,680. Any suitable tumor sample may be used, e.g., a formalin-fixed and paraffin-embedded (FFPE) tumor sample, an archival tumor sample, a fresh tumor sample, or a frozen tumor sample.

In some examples, assessment of the presence and/or expression level of PD-L1, and/or patient selection, may involve the use of two distinct affinity histochemical (AHC) assays (e.g., IHC assays) for PD-L1 protein: (a) an immune-directed PD-L1 assay; and (b) an immune-agnostic PD-L1 assay.

As described herein, an immune-directed PD-L1 assay is any AHC assay specific for human PD-L1 protein that has been designed to highlight immune cell expression of PD-L1, for example, by preferentially staining PD-L1-expressing immune cells versus PD-L1-expressing tumor cells. The highlighting of the immune cells may be a result of (a) inherent antibody specificity for immune-expressed PD-L1 versus expression by other cell types; (b) careful selection of staining conditions, such as antigen retrieval process, antibody diluent selection, buffer selection, detection system, labeling time and temperature, etc.; or (c) a combination of (a) and (b). An example of a commercially available immune-directed PD-L1 assay is the VENTANA PD-L1 (SP142) Assay (“SP142 Assay”). The SP142 Assay is an affinity histochemical assay that uses: (a) a PD-L1 rabbit monoclonal antibody (clone SP142, see U.S. Pat. No. 10,689,445); (b) an automated IHC/ISH staining platform (BENCHMARK IHC/ISH staining platform (Roche)); and (c) a tyramide-amplified 3,3′-diaminobenzedine (DAB)-based detection system (OPTIVIEW DAB IHC detection kit with OPTIVIEW Amplification kit (Roche)).

In an embodiment, the immune-directed PD-L1 assay is an AHC assay (e.g., an IHC assay) that has, for the given scoring algorithm and cutoff, at least 80% overall percent agreement (OPA), at least 80% positive percent agreement (PPA), and/or at least 80% negative percent agreement (NPA) with the SP142 Assay in the same indication and using the same scoring algorithm and cutoff. In a specific embodiment, the AHC has at least 80%, at least 85%, at least 90%, or at least 95% OPA with the SP142 assay. In another specific embodiment, the AHC has at least 80%, at least 85%, at least 90%, or at least 95% PPA with the SP142 assay. In another specific embodiment, the AHC has at least 80%, at least 85%, at least 90%, or at least 95% NPA with the SP142 assay. In another specific embodiment, the AHC has at least 80%, at least 85%, at least 90%, or at least 95% PPA with the SP142 Assay and at least 80%, at least 85%, at least 90%, or at least 95% NPA with the SP142 Assay. In an embodiment, the OPA, PPA, and/or NPA are measured using an immune proportion (IC) scoring method at a single cutoff that has been shown to be predictive for response to a PD-1 axis binding antagonist in the tested indication. In another embodiment, the OPA, PPA, and/or NPA are measured using a >5% IC 2/3 cutoff in a bladder cancer indication (e.g., locally advanced or metastatic UC).

As is also described herein, an immune-agnostic PD-L1 assay is any AHC assay (e.g., an IHC assay) specific for human PD-L1 protein that is not an immune-directed PD-L1 assay. Exemplary commercially-available immune-agnostic PD-L1 assays include the PD-L1 IHC 22C3 PHARMDX assay (Agilent) (hereafter, “22C3 Assay”), the VENTANA PD-L1 (SP263) Assay (Roche) (hereafter, “SP263 Assay”), and the PD-L1 IHC 28-8 PHARMDX assay (Agilent) (hereafter, “28-8 Assay”). In an embodiment, the immune-agnostic PD-L1 assay is an AHC assay (e.g., an IHC assay) that has at least 80% OPA, at least 80% PPA, and/or at least 80% NPA with one or more of the 22C3 Assay, the SP263 Assay, and the 28-8 Assay in the same indication and using the same scoring algorithm and cutoff. In a specific embodiment, the AHC has at least 80%, at least 85%, at least 90%, or at least 95% OPA with the 22C3 assay. In another specific embodiment, the AHC has at least 80%, at least 85%, at least 90%, or at least 95% PPA with the 22C3 assay. In another specific embodiment, the AHC has at least 80%, at least 85%, at least 90%, or at least 95% NPA with the 22C3 assay. In another specific embodiment, the AHC has at least 80%, at least 85%, at least 90%, or at least 95% PPA with the 22C3 assay and at least 80%, at least 85%, at least 90%, or at least 95% NPA with the 22C3 assay. In an embodiment, the OPA, PPA, and/or NPA are measured using a combined positive score (CPS) scoring method at a single cutoff that has been shown to be predictive for response to a PD-1 axis binding antagonist in the tested indication. In another embodiment, the OPA, PPA, and/or NPA are measured using a ≥10% CPS cutoff in a bladder cancer indication (e.g., locally advanced or metastatic UC).

In one example, provided herein is an assay for determining the presence or expression level of PD-L1 in a tumor sample obtained from a patient suffering from a cancer, the assay comprising: (a) determining the presence or expression level of PD-L1 in a tumor sample obtained from the patient using an immune-directed PD-L1 assay (e.g., the SP142 assay); and (b) determining the presence or expression level of PD-L1 in the tumor sample obtained from the patient using an immune-agnostic PD-L1 assay (e.g., the 22C3 Assay, the SP263 Assay or the 28-8 Assay).

In some examples, the assay is an AHC assay. In some examples, the AHC assay is an IHC assay.

For example, provided herein is an assay for determining the presence or expression level of PD-L1 in a tumor sample obtained from a patient suffering from a cancer, the assay comprising: (a) determining the presence or expression level of PD-L1 in a tumor sample obtained from the patient using a PD-L1 IHC assay comprising the VENTANA SP142 anti-PD-L1 diagnostic antibody; and (b) determining the presence or expression level of PD-L1 in the tumor sample obtained from the patient using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody.

Any suitable scoring algorithm may be used. For example, the scoring algorithm may be the immune cell scoring algorithm set forth in Table 2 herein, e.g., as used in an SP142 Assay. In some examples the scoring algorithm may be a Combined Positive Score (CPS), e.g., as used in a 22C3 Assay. It is to be understood that a CPS may be determined using other PD-L1 AHC assays (e.g., other PD-L1 IHC assays), e.g., an IHC assay comprising use of VENTANA SP263 or 28-8. Other scoring algorithms for PD-L1 assays are known in the art, e.g., TPS, percent of tumor cells (TC), and the tumor cell scoring algorithm set forth in Table 3 herein. A description of different exemplary scoring algorithms for PD-L1 assays that may be used is shown in FIG. 1 of Zajac et al. Diagnostic Pathology.

Any suitable cutoff may be used. For example, in some examples, the cutoff is IC ≥5%, e.g., as described in Table 2 herein. In some examples, the cutoff is CPS ≥1 or CPS ≥10. In some examples, the cutoff is CPS ≥10.

In some examples, the tumor sample obtained from the patient has a detectable expression level of PD-L1 in tumor-infiltrating immune cells that comprise 5% or more of the tumor sample using a PD-L1 IHC assay comprising the VENTANA SP142 anti-PD-L1 diagnostic antibody.

In some examples, the tumor sample obtained from the patient has the presence of discernible PD-L1 staining of any intensity in tumor-infiltrating immune cells covering ≥5% of tumor area occupied by tumor cells, associated intratumoral, and contiguous peritumoral stroma, as determined by the PD-L1 IHC assay comprising the VENTANA SP142 anti-PD-L1 diagnostic antibody.

In some examples, the tumor sample obtained from the patient has a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody. In some examples, the tumor sample obtained from the patient has a CPS of ≥10 using a PD-L1 IHC assay comprising the Dako 22C3 anti-PD-L1 diagnostic antibody.

In some examples, the tumor sample obtained from the patient has: a detectable expression level of PD-L1 in tumor-infiltrating immune cells that comprise 5% or more of the tumor sample using the PD-L1 IHC assay comprising the VENTANA SP142 anti-PD-L1 diagnostic antibody; and a CPS of ≥10 using the PD-L1 IHC assay comprising the Dako 22C3 anti-PD-L1 diagnostic antibody.

In some examples, steps (a) and (b) are performed simultaneously.

In some examples, steps (a) and (b) are performed sequentially.

In some examples, steps (a) and (b) are performed in different sections of the tumor sample or in the same section of the tumor sample.

In some examples, the different sections of the tumor sample are consecutive sections.

In some examples, the cancer is locally advanced or metastatic urothelial carcinoma.

In some examples, the patient is previously untreated for the locally advanced or metastatic urothelial carcinoma.

In some examples, the assay is used for (i) selecting a therapy for treating a locally advanced or metastatic UC in a patient in need thereof or (ii) identifying a patient having a locally advanced or metastatic UC who may benefit from a treatment regimen comprising an anti-PD-L1 antibody.

In some examples, the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8). In some examples, the anti-PD-L1 antibody is atezolizumab.

In some examples, the tumor sample is an FFPE tumor sample, an archival tumor sample, a fresh tumor sample, or a frozen tumor sample.

In another example, provided herein is a method of labeling PD-L1 in a tumor sample, the method comprising the following steps: (a) performing an immune-directed PD-L1 assay on the tumor sample; and (b) performing an immune-agnostic PD-L1 assay on the tumor sample.

Any suitable immune-directed PD-L1 assay may be used, e.g., the SP142 Assay.

Any suitable immune-agnostic PD-L1 assay may be used, e.g., the 22C3 Assay, the SP263 Assay, or the 28-8 Assay.

In another example, provided herein is a method of labeling PD-L1 in a tumor sample, the method comprising the following steps: (a) contacting the tumor sample with the VENTANA SP142 anti-PD-L1 diagnostic antibody; (b) contacting the tumor sample with the Dako 22C3 anti-PD-L1 diagnostic antibody, the VENTANA SP263 anti-PD-L1 diagnostic antibody, or the 28-8 anti-PD-L1 diagnostic antibody; and (c) visualizing the anti-PD-L1 diagnostic antibodies of steps (a) and (b) with one or more detectable reagents that generates a detectable signal for both of the anti-PD-L1 diagnostic antibodies.

In some examples, the detectable signal for the VENTANA SP142 anti-PD-L1 diagnostic antibody is an amplified signal.

In some examples, the amplified signal is generated by tyramide signal amplification.

In some examples, steps (a) and (b) are performed simultaneously.

In some examples, steps (a) and (b) are performed sequentially.

In some examples, steps (a) and (b) are performed in different sections of the tumor sample or in the same section of the tumor sample.

In other examples, the different sections of the tumor sample are consecutive sections.

In some examples, the visualizing comprises AHC.

In some examples, the visualizing comprises IHC or immunofluorescence (IF).

In some examples, the visualizing comprises IHC.

In some examples, the tumor sample is an FFPE tumor sample, an archival tumor sample, a fresh tumor sample, or a frozen tumor sample.

In some examples, the tumor sample is obtained from a patient having a cancer.

In some examples, the cancer is locally advanced or metastatic urothelial carcinoma.

In some examples, the patient is previously untreated for the locally advanced or metastatic urothelial carcinoma.

Any suitable scoring algorithm or approach may be used to assess the presence and/or expression level of PD-L1 in a biological sample (e.g., a tumor sample).

For example, the scoring algorithm may be the immune cell scoring algorithm set forth in Table 2 herein, e.g., as used in an SP142 Assay. In some examples the scoring algorithm may be a Combined Positive Score (CPS), e.g., as used in a 22C3 Assay. It is to be understood that a CPS may be determined using other PD-L1 AHC assays (e.g., other PD-L1 IHC assays), e.g., an IHC assay comprising use of VENTANA SP263 or 28-8. Other scoring algorithms for PD-L1 assays are known in the art, e.g., TPS, percent of tumor cells (TC), and the tumor cell scoring algorithm set forth in Table 3 herein. A description of different exemplary scoring algorithms for PD-L1 assays that may be used is shown in FIG. 1 of Zajac et al. Diagnostic Pathology.

In one example, PD-L1 expression may be determined in terms of the percentage of a tumor sample comprised by tumor-infiltrating immune cells expressing a detectable expression level of PD-L1, as the percentage of tumor-infiltrating immune cells in a tumor sample expressing a detectable expression level of PD-L1, and/or as the percentage of tumor cells in a tumor sample expressing a detectable expression level of PD-L1. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the patient, for example, as assessed by IHC using an anti-PD-L1 antibody (e.g., the SP142 antibody). Any suitable anti-PD-L1 antibody may be used, including, e.g., SP142 (Ventana), SP263 (Ventana), 22C3 (Dako), 28-8 (Dako), E1L3N (Cell Signaling Technology), 4059 (ProSci, Inc.), h5H1 (Advanced Cell Diagnostics), and 9A11. In some examples, the anti-PD-L1 antibody is SP142. In other examples, the anti-PD-L1 antibody is SP263.

In some examples, a tumor sample obtained from the patient has a detectable expression level of PD-L1 in less than 1% of the tumor cells in the tumor sample, in 1% or more of the tumor cells in the tumor sample, in from 1% to less than 5% of the tumor cells in the tumor sample, in 5% or more of the tumor cells in the tumor sample, in from 5% to less than 50% of the tumor cells in the tumor sample, or in 50% or more of the tumor cells in the tumor sample.

In some examples, a tumor sample obtained from the patient has a detectable expression level of PD-L1 in tumor-infiltrating immune cells that comprise less than 1% of the tumor sample, more than 1% of the tumor sample, from 1% to less than 5% of the tumor sample, more than 5% of the tumor sample, from 5% to less than 10% of the tumor sample, or more than 10% of the tumor sample.

In some examples, tumor samples may be scored for PD-L1 positivity in tumor-infiltrating immune cells and/or in tumor cells according to the criteria for diagnostic assessment shown in Table 2 and/or Table 3, respectively. In some examples, tumor samples may be scored for PD-L1 positivity in tumor-infiltrating immune cells and/or in tumor cells according to the criteria for diagnostic assessment shown in Table 2 and/or Table 3, respectively, for a PD-L1 IHC assay comprising the VENTANA SP142 anti-PD-L1 diagnostic antibody (e.g., the SP142 Assay).

In some embodiments, a tumor sample obtained from the subject has been determined to have a detectable expression level of PD-L1 in tumor-infiltrating immune cells that comprise about 1% or more (e.g., about 1% or more, 2% or more, 3% or more, 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, about 50% or more, about 60% or more, about 70% or more, about 80% or more, about 90% or more, about 95% or more, about 96% or more, about 97% or more, about 98% or more, about 99% or more, or 100%) of the tumor sample. For example, in some embodiments, a tumor sample obtained from the subject has been determined to have a detectable expression level of PD-L1 in tumor-infiltrating immune cells that comprise from about 1% to less than about 5% (e.g., from 1% to 4.9%, from 1% to 4.5%, from 1% to 4%, from 1% to 3.5%, from 1% to 3%, from 1% to 2.5%, or from 1% to 2%) of the tumor sample.

In some embodiments, a tumor sample obtained from the subject has been determined to have a detectable expression level of PD-L1 in about 1% or more (e.g., about 1% or more, 2% or more, 3% or more, 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, about 50% or more, about 60% or more, about 70% or more, about 80% or more, about 90% or more, about 95% or more, about 96% or more, about 97% or more, about 98% or more, about 99% or more, or 100%) of the tumor-infiltrating immune cells in the tumor sample. For example, in some embodiments, a tumor sample obtained from the subject has been determined to have a detectable expression level of PD-L1 in from about 1% to less than about 5% (e.g., from 1% to 4.9%, from 1% to 4.5%, from 1% to 4%, from 1% to 3.5%, from 1% to 3%, from 1% to 2.5%, or from 1% to 2%) of the tumor-infiltrating immune cells in the tumor sample.

In other embodiments, a tumor sample obtained from the subject has been determined to have a detectable expression level of PD-L1 in tumor-infiltrating immune cells that comprise about 5% or more of the tumor sample. For example, in some embodiments, a tumor sample obtained from the subject has been determined to have a detectable expression level of PD-L1 in tumor-infiltrating immune cells that comprise from about 5% to less than about 10% (e.g., from 5% to 9.5%, from 5% to 9%, from 5% to 8.5%, from 5% to 8%, from 5% to 7.5%, from 5% to 7%, from 5% to 6.5%, from 5% to 6%, from 5% to 5.5%, from 6% to 9.5%, from 6% to 9%, from 6% to 8.5%, from 6% to 8%, from 6% to 7.5%, from 6% to 7%, from 6% to 6.5%, from 7% to 9.5%, from 7% to 9%, from 7% to 7.5%, from 8% to 9.5%, from 8% to 9%, or from 8% to 8.5%) of the tumor sample.

In yet other embodiments, a tumor sample obtained from the subject has been determined to have a detectable expression level of PD-L1 in about 5% or more of the tumor-infiltrating immune cells in the tumor sample. For example, in some embodiments, a tumor sample obtained from the subject has been determined to have a detectable expression level of PD-L1 in from about 5% to less than about 10% (e.g., from 5% to 9.5%, from 5% to 9%, from 5% to 8.5%, from 5% to 8%, from 5% to 7.5%, from 5% to 7%, from 5% to 6.5%, from 5% to 6%, from 5% to 5.5%, from 6% to 9.5%, from 6% to 9%, from 6% to 8.5%, from 6% to 8%, from 6% to 7.5%, from 6% to 7%, from 6% to 6.5%, from 7% to 9.5%, from 7% to 9%, from 7% to 7.5%, from 8% to 9.5%, from 8% to 9%, or from 8% to 8.5%) of the tumor-infiltrating immune cells in the tumor sample.

In still further embodiments, a tumor sample obtained from the subject has been determined to have a detectable expression level of PD-L1 in tumor-infiltrating immune cells that comprise about 10% or more (e.g., 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 60% or more, 70% or more, 80% or more, 90% or more, 95% or more, 96% or more, 97% or more, 98% or more, 99% or more, or 100%) of the tumor sample.

In still further embodiments, a tumor sample obtained from the subject has been determined to have a detectable expression level of PD-L1 in about 10% or more (e.g., 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 60% or more, 70% or more, 80% or more, 90% or more, 95% or more, 96% or more, 97% or more, 98% or more, 99% or more, or 100%) of the tumor-infiltrating immune cells in the tumor sample.

In yet other embodiments, a tumor sample obtained from the subject has been determined to have a detectable expression level of PD-L1 in about 50% or more (e.g., about 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more) of the tumor cells in the tumor sample and/or a detectable expression level of PD-L1 in tumor-infiltrating immune cells that comprise about 10% or more (e.g., 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 60% or more, 70% or more, 80% or more, 90% or more, 95% or more, 96% or more, 97% or more, 98% or more, 99% or more, or 100%) of the tumor sample.

It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the subject, for example, as assessed by IHC using an anti-PD-L1 antibody (e.g., the SP142 antibody). Any suitable anti-PD-L1 antibody may be used, including, e.g., SP142 (Ventana), SP263 (Ventana), 22C3 (Dako), 28-8 (Dako), E1L3N (Cell Signaling Technology), 4059 (ProSci, Inc.), h5H1 (Advanced Cell Diagnostics), and 9A11. In some embodiments, the anti-PD-L1 antibody is SP142. In some embodiments, the anti-PD-L1 antibody is SP263.

In some embodiments, a tumor sample obtained from the subject has been determined to have a detectable expression level of PD-L1 in about 1% or more (e.g., about 1% or more, 2% or more, 3% or more, 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more) of the tumor cells in the tumor sample. For example, in some embodiments, a tumor sample obtained from the subject has been determined to have a detectable expression level of PD-L1 in from about 1% to less than about 5% (e.g., from 1% to 4.9%, from 1% to 4.5%, from 1% to 4%, from 1% to 3.5%, from 1% to 3%, from 1% to 2.5%, or from 1% to 2%) of the tumor cells in the tumor sample. In other embodiments, a tumor sample obtained from the subject has been determined to have a detectable expression level of PD-L1 in less than about 1% of the tumor cells in the tumor sample.

In other embodiments, a tumor sample obtained from the subject has been determined to have a detectable expression level of PD-L1 in about 5% or more of the tumor cells in the tumor sample. For example, in some embodiments, a tumor sample obtained from the subject has been determined to have a detectable expression level of PD-L1 in from about 5% to less than 50% (e.g., from 5% to 49.5%, from 5% to 45%, from 5% to 40%, from 5% to 35%, from 5% to 30%, from 5% to 25%, from 5% to 20%, from 5% to 15%, from 5% to 10%, from 5% to 9%, from 5% to 8%, from 5% to 7%, from 5% to 6%, from 10% to 49.5%, from 10% to 40%, from 10% to 35%, from 10% to 30%, from 10% to 25%, from 10% to 20%, from 10% to 15%, from 15% to 49.5%, from 15% to 45%, from 15% to 40%, from 15% to 35%, from 15% to 30%, from 15% to 30%, from 15% to 25%, from 15% to 20%, from 20% to 49.5%, from 20% to 45%, from 20% to 40%, from 20% to 35%, from 20% to 30%, from 20% to 25%, from 25% to 49.5%, from 25% to 45%, from 25% to 40%, from 25% to 35%, from 25% to 30%, from 30% to 49.5%, from 30% to 45%, from 30% to 40%, from 30% to 35%, from 35% to 49.5%, from 35% to 45%, from 35% to 40%, from 40% to 49.5%, from 40% to 45%, or from 45% to 49.5%) of the tumor cells in the tumor sample.

In yet other embodiments, a tumor sample obtained from the subject has been determined to have a detectable expression level of PD-L1 in about 50% or more (e.g., about 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more) of the tumor cells in the tumor sample. In some embodiments, a tumor sample obtained from the subject has been determined to have a detectable expression level of PD-L1 in from about 50% to about 99% (e.g., from 50% to 99%, from 50% to 95%, from 50% to 90%, from 50% to 85%, from 50% to 80%, from 50% to 75%, from 50% to 70%, from 50% to 65%, from 50% to 60%, from 50% to 55%, from 55% to 99%, from 55% to 95%, from 55% to 90%, from 55% to 85%, from 55% to 80%, from 55% to 75%, from 55% to 70%, from 55% to 65%, from 55% to 60%, from 60% to 99%, from 60% to 95%, from 60% to 90%, from 60% to 85%, from 60% to 80%, from 60% to 75%, from 60% to 70%, from 60% to 65%, from 65% to 99%, from 65% to 95%, from 65% to 90%, from 65% to 85%, from 65% to 80%, from 65% to 75%, from 65% to 70%, from 70% to 99%, from 70% to 95%, from 70% to 90%, from 70% to 85%, from 70% to 80%, from 70% to 75%, from 75% to 99%, from 75% to 95%, from 75% to 90%, from 75% to 85%, from 75% to 80%, from 80% to 99%, from 80% to 95%, from 80% to 90%, from 80% to 85%, from 85% to 99%, from 85% to 95%, from 85% to 90%, from 90% to 99%, or from 90% to 95%) of the tumor cells in the tumor sample.

In some instances, a CPS is determined in a tumor sample obtained from the patient. In some embodiments, the CPS is determined by positive staining with an anti-PD-L1 diagnostic antibody, wherein the anti-PD-L1 diagnostic antibody is SP142, SP263, 22C3, or 28-8 (e.g., as part of an IHC assay). In some embodiments, the CPS is greater than or equal to 1, as determined by positive staining with the anti-PD-L1 diagnostic antibody SP263 (e.g., as calculated using the Ventana SP263 IHC assay), 22C3 (e.g., as calculated using the PHARMDX 22C3 IHC assay), or 28-8 (e.g., as calculated using the PHARMDX 28-8 IHC assay). In some embodiments, the CPS is greater than or equal to 10, as determined by positive staining with an anti-PD-L1 antibody SP263 (e.g., as calculated using the Ventana SP263 IHC assay), 22C3 (e.g., as calculated using the PHARMDX 22C3 IHC assay), or 28-8 (e.g., as calculated using the PHARMDX 28-8 IHC assay).

In some instances, a PD-L1-positive tumor cell fraction of the subject is determined. In some embodiments, the PD-L1-positive tumor cell fraction is determined by positive staining with an anti-PD-L1 diagnostic antibody, wherein the anti-PD-L1 antibody is SP142, SP263, 22C3, or 28-8 (e.g., as part of an IHC assay). In some embodiments, the PD-L1-positive tumor cell fraction is greater than or equal to 1% tumor cell (TC), as determined by positive staining with an anti-PD-L1 antibody SP263 (e.g., as calculated using the Ventana SP263 IHC assay) or 22C3 (e.g., as calculated using the PHARMDX 22C3 IHC assay). In some embodiments, the PD-L1-positive tumor cell fraction is less than 1% TC (e.g., from 0% to 1% TC, e.g., PD-L1-negative), as determined by positive staining with an anti-PD-L1 antibody SP263 (e.g., as calculated using the Ventana SP263 IHC assay) or 22C3 (e.g., as calculated using the PHARMDX 22C3 IHC assay).

In some instances, in any of the methods, uses, or compositions for use described herein, a tumor sample obtained from the individual has a detectable nucleic acid expression level of PD-L1. In some instances, the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof.

In some instances, the sample is selected from the group consisting of a tissue sample, a whole blood sample, a serum sample, and a plasma sample.

In some instances, the tissue sample is a tumor sample. Any suitable tumor sample may be used. In some instances, the tumor sample comprises tumor-infiltrating immune cells, tumor cells, stromal cells, and any combinations thereof.

In some embodiments, the tumor sample is a formalin-fixed and paraffin-embedded (FFPE) tumor sample, an archival tumor sample, a fresh tumor sample, or a frozen tumor sample. In some embodiments, the tumor sample is an FFPE tumor sample.

In other instances, the sample may be a cytology sample (e.g., a fine needle aspirate).

The presence and/or expression level of any of the biomarkers described above (including PD-L1 (e.g., PD-L1 expression on tumor-infiltrating immune cells (IC) in a tumor sample obtained from the subject and/or PD-L1 expression on tumor cells (TC) in a tumor sample obtained from the subject)), e.g., in a tumor sample obtained from the subject) may be assessed qualitatively and/or quantitatively based on any suitable criterion known in the art, including but not limited to DNA, RNA, cDNA, proteins, protein fragments, and/or gene copy number. Methodologies for measuring such biomarkers are known in the art and understood by the skilled artisan, including, but not limited to, IHC, Western blot analysis, immunoprecipitation, molecular binding assays, ELISA, ELIFA, fluorescence activated cell sorting (“FACS”), MassARRAY, proteomics, quantitative blood based assays (e.g., Serum ELISA), biochemical enzymatic activity assays, in situ hybridization (ISH), fluorescence in situ hybridization (FISH), Southern analysis, Northern analysis, whole genome sequencing, polymerase chain reaction (PCR) including quantitative real time PCR (qRT-PCR) and other amplification type detection methods, such as, for example, branched DNA, SISBA, TMA and the like, RNASeq, microarray analysis, gene expression profiling, whole-genome sequencing (WGS), and/or serial analysis of gene expression (“SAGE”), as well as any one of the wide variety of assays that can be performed by protein, gene, and/or tissue array analysis. Typical protocols for evaluating the status of genes and gene products are found, for example, in Ausubel et al. eds. (Current Protocols in Molecular Biology, 1995), Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

In some embodiments of any of the preceding methods, the expression level of a biomarker (e.g., PD-L1) may be a protein expression level.

Biomarker analysis can be performed, e.g., by AHC or ACC methods. AHC and ACC methods are typically accomplished by contacting a sample from the tumor with a biomarker-specific reagent under conditions that facilitate specific binding between the biomarker and the biomarker-specific reagent. The sample is then contacted with a set of detection reagents that interact with the biomarker-specific reagent to facilitate deposition a detectable moiety in close proximity the biomarker, thereby generating a detectable signal localized to the biomarker. Typically, wash steps are performed between application of different reagents to prevent unwanted non-specific labeling of tissues. Biomarker-labeled samples may optionally be additionally labeled with a contrast agent to visualize macromolecular structures.

The samples used for the AHC/ACC assay are typically tissue samples processed in a manner compatible with affinity labeling and brightfield microscopic analysis of the sample. In a specific embodiment, the sample is a microtome section of a formalin-fixed, paraffin-embedded (FFPE) sample derived from a tumor.

Exemplary biomarker-specific reagents useful for AHC and ACC methods include antibodies and antigen binding fragments thereof, ADNECTINs (scaffold based on 10th FN3 fibronectin; Bristol-Myers-Squibb Co.), AFFIBODYs (scaffold based on Z domain of protein A from S. aureus; Affibody AB, Solna, Sweden), AVIMERs (scaffold based on domain A/LDL receptor; Amgen, Thousand Oaks, CA), dAbs (scaffold based on VH or VL antibody domain; GlaxoSmithKline PLC, Cambridge, UK), DARPins (scaffold based on Ankyrin repeat proteins; Molecular Partners AG, Zürich, CH), ANTICALINs (scaffold based on lipocalins; Pieris AG, Freising, DE), NANOBODYs (scaffold based on VHH (camelid Ig); Ablynx N/V, Ghent, BE), TRANS-BODYs (scaffold based on Transferrin; Pfizer Inc., New York, NY), SMIPs (Emergent Biosolutions, Inc., Rockville, MD), and TETRANECTINs (scaffold based on C-type lectin domain (CTLD), tetranectin; Borean Pharma A/S, Aarhus, DK). Such biomarker-specific reagents are reviewed by Wurch et al., Development of Novel Protein Scaffolds as Alternatives to Whole Antibodies for Imaging and Therapy: Status on Discovery Research and Clinical Validation, Current Pharmaceutical Biotechnology, Vol. 9, pp. 502-509 (2008), the content of which is incorporated by reference.

Non-limiting examples of commercially available detection reagents or kits comprising detection reagents suitable for use with present methods include: VENTANA ULTRAVIEW detection systems (secondary antibodies conjugated to enzymes, including HRP and AP); VENTANA IVIEW detection systems (biotinylated anti-species secondary antibodies and streptavidin-conjugated enzymes); VENTANA Amplification kit (unconjugated secondary antibodies, which can be used with any of the foregoing VENTANA detection systems to increase the number of enzymes deposited at the site of primary antibody binding); OPTIVIEW detection systems (anti-species secondary antibody conjugated to a hapten and an anti-hapten tertiary antibody conjugated to an enzyme multimer); OPTIVIEW Amplification system (Anti-species secondary antibody conjugated to a hapten, an anti-hapten tertiary antibody conjugated to an enzyme multimer, and a tyramide conjugated to the same hapten, which can be used with the OPTIVIEW kit to increase the number of enzymes deposited at the site of primary antibody binding); POWERVISION and POWERVISION+ IHC Detection Systems (secondary antibodies directly polymerized with HRP or AP into compact polymers bearing a high ratio of enzymes to antibodies); DAKO ENVISION™+ System (enzyme labeled polymer that is conjugated to secondary antibodies); ULTRAPLEX Multiplex Chromogenic IHC Technology from CELL IDx (hapten-labeled primary antibodies combined with enzyme-labeled or fluor-labeled anti-hapten secondary antibodies).

If desired, the biomarker-labeled slides may be counterstained to assist in identifying morphologically relevant areas for identifying ROIs, either manually or automatically. Examples of counterstains include brightfield nuclear counterstains, such as hematoxylin (stains from blue to violet), Methylene blue (stains blue), toluidine blue (stains nuclei deep blue and polysaccharides pink to red), nuclear fast red (also called Kernechtrot dye, stains red), and methyl green (stains green) and non-nuclear chromogenic stains, such as eosin (stains pink).

The AHC/ACC assay and counterstain may be applied to the sample using an automated labeling system. Prichard, Overview of Automated Immunohistochemistry, Arch Pathol Lab Med., Vol. 138, pp. 1578-1582 (2014), incorporated herein by reference in its entirety, describes several specific examples of automated AHC labeling systems and their various features, including the intelliPATH (Biocare Medical), WAVE (Celerus Diagnostics), DAKO OMNIS and DAKO AUTOSTAINER LINK 48 (Agilent Technologies), BENCHMARK (Ventana Medical Systems, Inc.), Leica BOND, and LAB VISION AUTOSTAINER (Thermo Scientific) automated AHC labeling systems. Commercially-available labeling units typically operate on one of the following principles: (1) open individual slide labeling, in which slides are positioned horizontally and reagents are dispensed as a puddle on the surface of the slide containing a tissue sample (such as implemented on the DAKO AUTOSTAINER Link 48 (Agilent Technologies) and INTELLIPATH (Biocare Medical) labelers); (2) liquid overlay technology, in which reagents are either covered with or dispensed through an inert fluid layer deposited over the sample (such as implemented on BENCHMARK labelers); (3) capillary gap labeling, in which the slide surface is placed in proximity to another surface to create a narrow gap, through which capillary forces draw up and keep liquid reagents in contact with the samples (such as the labeling principles used by DAKO TECHMATE, Leica BOND, and DAKO OMNIS labelers). Some iterations of capillary gap labeling do not mix the fluids in the gap (such as on the DAKO TECHMATE and the Leica BOND). In variations of capillary gap labeling termed dynamic gap labeling, capillary forces are used to apply sample to the slide, and then the parallel surfaces are translated relative to one another to agitate the reagents during incubation to effect reagent mixing (such as the labeling principles implemented on DAKO OMNIS slide labelers (Agilent)). It has also been proposed to use inkjet technology to deposit reagents on slides. See WO 2016/170008 A1. This list of labeling technologies is not intended to be comprehensive, and any fully or semi-automated system or manual method for performing biomarker labeling may be incorporated into the present methods.

In certain embodiments, the method comprises contacting the sample with antibodies that specifically bind to a biomarker described herein under conditions permissive for binding of the biomarker, and detecting whether a complex is formed between the antibodies and biomarker. Such method may be an in vitro or in vivo method. In some embodiments, an antibody is used to select subjects eligible for treatment with an anti-cancer therapy that includes a PD-1 axis binding antagonist, e.g., an anti-PD-L1 antibody (e.g., atezolizumab), e.g., a biomarker for selection of subjects. In some embodiments, an antibody is used to select subjects eligible for treatment with an anti-cancer therapy that includes a PD-1 axis binding antagonist (e.g., an anti-PD-L1 antibody (e.g., atezolizumab)) and/or a platinum-based chemotherapy (e.g., cisplatin or carboplatin and gemcitabine), e.g., a biomarker for selection of subjects.

Any method of measuring protein expression levels known in the art or provided herein may be used. For example, in some embodiments, a protein expression level of a biomarker is determined using a method selected from the group consisting of immunohistochemistry (IHC), flow cytometry (e.g., fluorescence-activated cell sorting (FACS™)), Western blot, enzyme-linked immunosorbent assay (ELISA), immunoprecipitation, immunofluorescence, radioimmunoassay, dot blotting, immunodetection methods, HPLC, surface plasmon resonance, optical spectroscopy, mass spectrometry, and HPLC.

In some embodiments, the protein expression level of the biomarker (e.g., PD-L1) is determined in tumor-infiltrating immune cells. In some embodiments, the protein expression level of the biomarker is determined in tumor cells. In some embodiments, the protein expression level of the biomarker is determined in tumor-infiltrating immune cells and/or in tumor cells. In some embodiments, the protein expression level of the biomarker is determined in peripheral blood mononuclear cells (PBMCs).

In certain embodiments, the presence and/or expression level/amount of a biomarker protein (e.g., PD-L1) in a sample is examined using IHC and staining protocols. IHC staining of tissue sections has been shown to be a reliable method of determining or detecting the presence of proteins in a sample. In some embodiments of any of the methods, assays and/or kits, the biomarker is one or more of the protein expression products of PD-L1. In one embodiment, an expression level of biomarker is determined using a method comprising: (a) performing IHC analysis of a sample (such as a tumor sample obtained from a subject) with an antibody; and (b) determining expression level of a biomarker in the sample. In some embodiments, IHC staining intensity is determined relative to a reference. In some embodiments, the reference is a reference value. In some embodiments, the reference is a reference sample (e.g., a control cell line staining sample, a tissue sample from non-cancerous subject, or a tumor sample that is determined to be negative for the biomarker of interest).

For example, in some embodiments, the protein expression level of PD-L1 is determined using IHC. In some embodiments, the protein expression level of PD-L1 is detected using an anti-PD-L1 antibody. Any suitable anti-PD-L1 antibody may be used, including, e.g., SP142, SP263, 22C3, 28-8, E1L3N, 4059, h5H1, and 9A11. In some embodiments, the anti-PD-L1 antibody is SP142. In some embodiments, the anti-PD-L1 antibody is SP263.

IHC may be performed in combination with additional techniques such as morphological staining and/or in situ hybridization (e.g., ISH). Two general methods of IHC are available; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for IHC typically will be labeled with a detectable moiety. Numerous labels are available which can be generally grouped into the following categories: (a) radioisotopes, such as ³⁵S, ¹⁴C, ¹²⁵I, ³H, and ¹³¹I; (b) colloidal gold particles; (c) fluorescent labels including, but are not limited to, rare earth chelates (europium chelates), Texas Red, rhodamine, fluorescein, dansyl, lissamine, umbelliferone, phycocrytherin, phycocyanin, or commercially-available fluorophores such as SPECTRUM ORANGE7 and SPECTRUM GREEN7 and/or derivatives of any one or more of the above; (d) various enzyme-substrate labels are available and U.S. Pat. No. 4,275,149 provides a review of some of these. Examples of enzymatic labels include luciferases (e.g., firefly luciferase and bacterial luciferase; see, e.g., U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

Examples of enzyme-substrate combinations include, for example, horseradish peroxidase (HRPO) with hydrogen peroxidase as a substrate; alkaline phosphatase (AP) with para-Nitrophenyl phosphate as chromogenic substrate; and β-D-galactosidase (β-D-Gal) with a chromogenic substrate (e.g., p-nitrophenyl-β-D-galactosidase) or fluorogenic substrate (e.g., 4-methylumbelliferyl-β-D-galactosidase). For a general review of these, see, for example, U.S. Pat. Nos. 4,275,149 and 4,318,980.

Specimens may be prepared, for example, manually, or using an automated staining instrument (e.g., a Ventana BenchMark XT or Benchmark ULTRA instrument). Specimens thus prepared may be mounted and coverslipped. Slide evaluation is then determined, for example, using a microscope, and staining intensity criteria, routinely used in the art, may be employed. In one embodiment, it is to be understood that when cells and/or tissue from a tumor is examined using IHC, staining can be determined or assessed in tumor cell(s) and/or tissue (as opposed to stromal or surrounding tissue that may be present in the sample). In other embodiments, staining can be determined or assessed in stromal or surrounding tissue that may be present in the sample. In some embodiments, it is understood that when cells and/or tissue from a tumor is examined using IHC, staining includes determining or assessing in tumor-infiltrating immune cells, including intratumoral or peritumoral immune cells. In some embodiments, the presence of a biomarker is detected by IHC in >0% of the sample, in at least 1% of the sample, in at least 5% of the sample, in at least 10% of the sample, in at least 15% of the sample, in at least 15% of the sample, in at least 20% of the sample, in at least 25% of the sample, in at least 30% of the sample, in at least 35% of the sample, in at least 40% of the sample, in at least 45% of the sample, in at least 50% of the sample, in at least 55% of the sample, in at least 60% of the sample, in at least 65% of the sample, in at least 70% of the sample, in at least 75% of the sample, in at least 80% of the sample, in at least 85% of the sample, in at least 90% of the sample, in at least 95% of the sample, or more. Samples may be scored using any method known in the art, for example, by a pathologist or automated image analysis.

In some embodiments of any of the methods, the biomarker is detected by immunohistochemistry using a diagnostic antibody (i.e., primary antibody). In some embodiments, the diagnostic antibody specifically binds human antigen. In some embodiments, the diagnostic antibody is a non-human antibody. In some embodiments, the diagnostic antibody is a rat, mouse, or rabbit antibody. In some embodiments, the diagnostic antibody is a rabbit antibody. In some embodiments, the diagnostic antibody is a monoclonal antibody. In some embodiments, the diagnostic antibody is directly labeled. In other embodiments, the diagnostic antibody is indirectly labeled (e.g., by a secondary antibody).

In other embodiments of any of the preceding methods, the expression level of a biomarker may be a nucleic acid expression level (e.g., a DNA expression level or an RNA expression level (e.g., an mRNA expression level)). Any suitable method of determining a nucleic acid expression level may be used. In some embodiments, the nucleic acid expression level is determined using RNAseq, RT-qPCR, qPCR, multiplex qPCR or RT-qPCR, microarray analysis, SAGE, MassARRAY technique, ISH, or a combination thereof.

Methods for the evaluation of mRNAs in cells are well known and include, for example, serial analysis of gene expression (SAGE), whole genome sequencing (WGS), hybridization assays using complementary DNA probes (such as in situ hybridization using labeled riboprobes specific for the one or more genes, Northern blot and related techniques) and various nucleic acid amplification assays (such as RT-PCR (e.g., qRT-PCR) using complementary primers specific for one or more of the genes, and other amplification type detection methods, such as, for example, branched DNA, SISBA, TMA and the like). In addition, such methods can include one or more steps that allow one to determine the levels of target mRNA in a biological sample (e.g., by simultaneously examining the levels a comparative control mRNA sequence of a “housekeeping” gene such as an actin family member). Optionally, the sequence of the amplified target cDNA can be determined. Optional methods include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlates with increased or reduced clinical benefit of treatment comprising an immunotherapy and a suppressive stromal antagonist may be arrayed on a solid support. Hybridization of a labeled probe with a particular array member indicates that the sample from which the probe was derived expresses that gene.

The sample may be obtained from the subject at any suitable time. For example, in some embodiments, the sample is obtained from the subject prior to (e.g., minutes, hours, days, weeks (e.g., 1, 2, 3, 4, 5, 6, or 7 weeks), months, or years prior to) administration of the treatment regimen. In some embodiments of any of the preceding methods, the sample from the subject is obtained about 2 to about 10 weeks (e.g., 2, 3, 4, 5, 6, 7, 8, 9, or 10 weeks) following administration of the treatment regimen. In some embodiments, the sample from the subject is obtained about 4 to about 6 weeks following administration of the treatment regimen.

In some embodiments, the expression level or number of a biomarker (e.g., PD-L1) is detected in a tissue sample, a primary or cultured cells or cell line, a cell supernatant, a cell lysate, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, or any combination thereof. In some embodiments, the sample is a tissue sample (e.g., a tumor tissue sample), a cell sample, a whole blood sample, a plasma sample, a serum sample, or a combination thereof. In some embodiments, the tumor tissue sample wherein the tumor tissue sample includes tumor cells, tumor-infiltrating immune cells, stromal cells, or a combination thereof. In some embodiments, the tumor tissue sample is a formalin-fixed and paraffin-embedded (FFPE) sample, an archival sample, a fresh sample, or a frozen sample.

In some examples, the sample may be a cytology sample (e.g., a fine needle aspirate).

For example, in some embodiments, the expression level of a biomarker (e.g., PD-L1) is detected in tumor-infiltrating immune cells, tumor cells, PBMCs, or combinations thereof using known techniques (e.g., IHC, immunofluorescence microscopy, or flow cytometry). Tumor-infiltrating immune cells include, but are not limited to, intratumoral immune cells, peritumoral immune cells or any combinations thereof, and other tumor stroma cells (e.g., fibroblasts). Such tumor infiltrating immune cells may be T lymphocytes (such as CD8+ T lymphocytes (e.g., CD8+ T effector (Teff) cells) and/or CD4+ T lymphocytes (e.g., CD4+ Teff cells), B lymphocytes, or other bone marrow-lineage cells including granulocytes (neutrophils, eosinophils, basophils), monocytes, macrophages, dendritic cells (e.g., interdigitating dendritic cells), histiocytes, and natural killer (NK) cells. In some embodiments, the staining for a biomarker is detected as membrane staining, cytoplasmic staining, or combinations thereof. In other embodiments, the absence of a biomarker is detected as absent or no staining in the sample, relative to a reference sample.

In particular embodiments, the expression level of a biomarker is assessed in a sample that contains or is suspected to contain cancer cells. The sample may be, for example, a tissue biopsy or a metastatic lesion obtained from a subject suffering from, suspected to suffer from, or diagnosed with cancer (e.g., bladder cancer (e.g., UC, including locally advanced or metastatic UC). In some embodiments, the sample is a sample of tissue (e.g., renal pelvis, ureter, urinary bladder, and/or urethral tissue), a biopsy of a tumor (e.g., a locally advanced or metastatic UC tumor, including a pelvis, ureter, urinary bladder, and/or urethral tumor), a known or suspected metastatic bladder cancer (e.g., metastatic UC) lesion or section, or a blood sample, e.g., a peripheral blood sample, known or suspected to comprise circulating cancer cells, e.g., bladder cancer cells (e.g., UC cells, including locally advanced or metastatic UC cells). The sample may comprise both cancer cells, i.e., tumor cells, and non-cancerous cells (e.g., lymphocytes, such as T cells or NK cells), and, in certain embodiments, comprises both cancerous and non-cancerous cells. Methods of obtaining biological samples including tissue resections, biopsies, and body fluids, e.g., blood samples comprising cancer/tumor cells, are well known in the art.

The sample may be obtained from a patient having any suitable cancer cancer (e.g., bladder cancer (e.g., UC, including mUC; MIBC, and NMIBC); kidney or renal cancer (e.g., RCC); lung cancer, including small-cell lung cancer, non-small cell lung cancer, adenocarcinoma of the lung, and squamous carcinoma of the lung; cancer of the urinary tract; breast cancer (e.g., HER2+ breast cancer and TNBC, which are ER−, PR−, and HER2 HER2−); prostate cancer, such as CRPC; cancer of the peritoneum; hepatocellular cancer; gastric or stomach cancer, including gastrointestinal cancer and gastrointestinal stromal cancer; pancreatic cancer (e.g., PDAC); glioblastoma; cervical cancer; ovarian cancer; liver cancer (e.g., HCC); hepatoma; colon cancer; rectal cancer; colorectal cancer; endometrial or uterine carcinoma; salivary gland carcinoma; prostate cancer; vulval cancer; thyroid cancer; hepatic carcinoma; anal carcinoma; penile carcinoma; melanoma, including superficial spreading melanoma, lentigo malignant melanoma, acral lentiginous melanomas, and nodular melanomas; multiple myeloma and B-cell lymphoma (including low grade/follicular NHL; SL NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); CLL; ALL; AML; hairy cell leukemia; CML; PTLD; and MDS, as well as abnormal vascular proliferation associated with phakomatoses, edema (such as that associated with brain tumors), Meigs' syndrome, brain cancer, head and neck cancer, and associated metastases). In some examples, the cancer is a bladder cancer (e.g., UC, including locally advanced or metastatic UC).

In certain embodiments, the subject may have an advanced, refractory, recurrent, and/or chemotherapy-resistant form of the cancer.

In certain embodiments, the presence and/or expression levels/amount of a biomarker in a first sample is increased or elevated as compared to presence/absence and/or expression levels/amount in a second sample. In certain embodiments, the presence/absence and/or expression levels/amount of a biomarker in a first sample is decreased or reduced as compared to presence and/or expression levels/amount in a second sample. In certain embodiments, the second sample is a reference sample, reference cell, reference tissue, control sample, control cell, or control tissue.

In certain embodiments, a reference sample, reference cell, reference tissue, control sample, control cell, or control tissue is a single sample or combined multiple samples from the same subject that are obtained at one or more different time points than when the test sample is obtained. For example, a reference sample, reference cell, reference tissue, control sample, control cell, or control tissue is obtained at an earlier time point from the same subject than when the test sample is obtained. Such reference sample, reference cell, reference tissue, control sample, control cell, or control tissue may be useful if the reference sample is obtained during initial diagnosis of cancer and the test sample is later obtained when the cancer becomes metastatic.

In certain embodiments, a reference sample, reference cell, reference tissue, control sample, control cell, or control tissue is a combined multiple samples from one or more healthy individuals who are not the subject. In certain embodiments, a reference sample, reference cell, reference tissue, control sample, control cell, or control tissue is a combined multiple samples from one or more individuals with a disease or disorder (e.g., cancer) who are not the subject. In certain embodiments, a reference sample, reference cell, reference tissue, control sample, control cell, or control tissue is pooled RNA samples from normal tissues or pooled plasma or serum samples from one or more individuals who are not the subject. In certain embodiments, a reference sample, reference cell, reference tissue, control sample, control cell, or control tissue is pooled RNA samples from tumor tissues or pooled plasma or serum samples from one or more individuals with a disease or disorder (e.g., cancer) who are not the subject.

In some embodiments, the method further includes administering an effective amount of a treatment regimen described herein (e.g., a treatment regimen comprising a PD-1 axis binding antagonist (e.g., an anti-PD-L1 antibody (e.g., atezolizumab)) and/or a platinum-based chemotherapy (e.g., cisplatin or carboplatin and gemcitabine) to the subject, for example, based on the expression level of one or more biomarkers (e.g., PD-L1). The treatment regimen may be any treatment regimen described herein, e.g., in Section II above.

The presence and/or expression level of PD-L1 may be assessed in a subject treated according to any of the methods and compositions for use described herein. In some embodiments, the method includes determining the expression level of PD-L1 in a biological sample (e.g., a tumor sample) obtained from the subject. In other embodiments, the expression level of PD-L1 in a biological sample (e.g., a tumor sample) obtained from the subject has been determined prior to initiation of treatment. In yet other embodiments, the expression level of PD-L1 in a biological sample (e.g., a tumor sample) obtained from the subject may be determined after initiation of treatment.

### V. PD-1 Axis Binding Antagonists

PD-1 axis binding antagonists may include PD-L1 binding antagonists, PD-1 binding antagonists, and PD-L2 binding antagonists. Any suitable PD-1 axis binding antagonist may be used.

Provided herein are methods for treating or delaying progression of bladder cancer (e.g., UC, including locally advanced or metastatic UC) in a subject comprising administering to the subject a treatment regimen comprising an effective amount of a PD-1 axis binding antagonist (e.g., an anti-PD-L1 antibody (e.g., atezolizumab)). Also provided herein are methods for treating or delaying progression of bladder cancer (e.g., UC, including locally advanced or metastatic UC) in a subject comprising administering to the subject a treatment regimen comprising an effective amount of a PD-1 axis binding antagonist (e.g., an anti-PD-L1 antibody (e.g., atezolizumab)) and a platinum-based chemotherapy (e.g., cisplatin or carboplatin and gemcitabine). Also provided herein are methods of enhancing immune function in a subject having a bladder cancer (e.g., UC, including locally advanced or metastatic UC) comprising administering to the subject a treatment regimen comprising an effective amount of a PD-1 axis binding antagonist (e.g., an anti-PD-L1 antibody (e.g., atezolizumab)). Further provided herein are methods of enhancing immune function in a subject having a bladder cancer (e.g., UC, including locally advanced or metastatic UC) comprising administering to the subject a treatment regimen comprising an effective amount of a PD-1 axis binding antagonist (e.g., an anti-PD-L1 antibody (e.g., atezolizumab)) and a platinum-based chemotherapy (e.g., cisplatin or carboplatin and gemcitabine). Also provided are related compositions (e.g., pharmaceutical compositions) for use, kits, and articles of manufacture. Any of the methods, compositions for use, kits, or articles of manufacture described herein may include or involve any of the PD-1 axis binding antagonists described below.

### A. PD-L1 Binding Antagonists

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. The PD-L1 binding antagonist may be, without limitation, an antibody, an antigen-binding fragment thereof, an immunoadhesin, a fusion protein, an oligopeptide, or a small molecule. In some instances, the PD-L1 binding antagonist is a small molecule that inhibits PD-L1 (e.g., GS-4224, INCB086550, MAX-10181, INCB090244, CA-170, or ABSK041). In some instances, the PD-L1 binding antagonist is a small molecule that inhibits PD-L1 and VISTA. In some instances, the PD-L1 binding antagonist is CA-170 (also known as AUPM-170). In some instances, the PD-L1 binding antagonist is a small molecule that inhibits PD-L1 and TIM3. In some instances, the small molecule is a compound described in WO 2015/033301 and/or WO 2015/033299.

In some instances, the PD-L1 binding antagonist is an anti-PD-L1 antibody. A variety of anti-PD-L1 antibodies are contemplated and described herein. In any of the instances herein, the isolated anti-PD-L1 antibody can bind to a human PD-L1, for example a human PD-L1 as shown in UniProtKB/Swiss-Prot Accession No. Q9NZQ7-1, or a variant thereof. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)₂ fragments. In some instances, the anti-PD-L1 antibody is a humanized antibody. In some instances, the anti-PD-L1 antibody is a human antibody. Exemplary anti-PD-L1 antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. Examples of anti-PD-L1 antibodies useful in the methods of this invention and methods of making them are described in International Patent Application Publication No. WO 2010/077634 and U.S. Pat. No. 8,217,149, each of which is incorporated herein by reference in its entirety.

In some instances, the anti-PD-L1 antibody comprises:
(a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and
(b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

In one embodiment, the anti-PD-L1 antibody comprises:

(a) a heavy chain variable region (VH) comprising the amino acid sequence:
(SEQ ID NO: 9)
EVQLVESGGGLVQPGGSLRLSCAASGFTFSDSWIHWVRQAPGKGLEWVAWISPYGGSTYYADSVKGRF
TISADTSKNTAYLQMNSLRAEDTAVYYCARRHWPGGFDYWGQGTLVTVSS,
and
(b) the light chain variable region (VL) comprising the amino acid sequence:
(SEQ ID NO: 10)
DIQMTQSPSSLSASVGDRVTITCRASQDVSTAVAWYQQKPGKAPKLLIYSASFLYSGVPSRFSGSGSGTD
FTLTISSLOPEDFATYYCQQYLYHPATFGQGTKVEIKR.

In some instances, the anti-PD-L1 antibody comprises (a) a VH comprising an amino acid sequence comprising having at least 95% sequence identity (e.g., at least 95%, 96%, 97%, 98%, or 99% sequence identity) to, or the sequence of SEQ ID NO: 9; (b) a VL comprising an amino acid sequence comprising having at least 95% sequence identity (e.g., at least 95%, 96%, 97%, 98%, or 99% sequence identity) to, or the sequence of SEQ ID NO: 10; or (c) a VH as in (a) and a VL as in (b).

In one embodiment, the anti-PD-L1 antibody comprises atezolizumab, which comprises:

(a) the heavy chain amino acid sequence:
(SEQ ID NO: 1)
EVQLVESGGGLVQPGGSLRLSCAASGFTFSDSWIHWVRQAPGKGLEWVAWISPYGGSTYYADSVKGRF
TISADTSKNTAYLQMNSLRAEDTAVYYCARRHWPGGFDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCDKTHTCPPCPAPELLGGPSVFLFPPKPKDTLMISRTPEVTCVVVDVSHEDPEVKFN
WYVDGVEVHNAKTKPREEQYASTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPG,
and
(b) the light chain amino acid sequence:
(SEQ ID NO: 2)
DIQMTQSPSSLSASVGDRVTITCRASQDVSTAVAWYQQKPGKAPKLLIYSASFLYSGVPSRFSGSGSGTD
FTLTISSLQPEDFATYYCQQYLYHPATFGQGTKVEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFN
RGEC.

In some instances, the anti-PD-L1 antibody is avelumab (CAS Registry Number: 1537032-82-8). Avelumab, also known as MSB0010718C, is a human monoclonal IgG1 anti-PD-L1 antibody (Merck KGaA, Pfizer).

In some instances, the anti-PD-L1 antibody is durvalumab (CAS Registry Number: 1428935-60-7). Durvalumab, also known as MEDI4736, is an Fc-optimized human monoclonal IgG1 kappa anti-PD-L1 antibody (MedImmune, AstraZeneca) described in WO 2011/066389 and US 2013/034559.

In some instances, the anti-PD-L1 antibody is MDX-1105 (Bristol Myers Squibb). MDX-1105, also known as BMS-936559, is an anti-PD-L1 antibody described in WO 2007/005874.

In some instances, the anti-PD-L1 antibody is LY3300054 (Eli Lilly).

In some instances, the anti-PD-L1 antibody is STI-A1014 (Sorrento). STI-A1014 is a human anti-PD-L1 antibody.

In some instances, the anti-PD-L1 antibody is KN035 (Suzhou Alphamab). KN035 is single-domain antibody (dAB) generated from a camel phage display library.

In some instances, the anti-PD-L1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some instances, the anti-PD-L1 antibody is CX-072 (CytomX Therapeutics).

In some instances, the anti-PD-L1 antibody comprises the six HVR sequences (e.g., the three heavy chain HVRs and the three light chain HVRs) and/or the heavy chain variable domain and light chain variable domain from an anti-PD-L1 antibody described in US20160108123, WO 2016/000619, WO 2012/145493, U.S. Pat. No. 9,205,148, WO 2013/181634, or WO 2016/061142.

In a still further specific aspect, the anti-PD-L1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In still a further instance, the effector-less Fc mutation is an N297A substitution in the constant region. In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Removal of glycosylation sites from an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site with another amino acid residue (e.g., glycine, alanine, or a conservative substitution).

### B. PD-1 Binding Antagonists

In some instances, the PD-1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. The PD-1 binding antagonist may be, without limitation, an antibody, an antigen-binding fragment thereof, an immunoadhesin, a fusion protein, an oligopeptide, or a small molecule. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). For example, in some instances, the PD-1 binding antagonist is an Fc-fusion protein. In some instances, the PD-1 binding antagonist is AMP-224. AMP-224, also known as B7-DCIg, is a PD-L2-Fc fusion soluble receptor described in WO 2010/027827 and WO 2011/066342. In some instances, the PD-1 binding antagonist is a peptide or small molecule compound. In some instances, the PD-1 binding antagonist is AUNP-12 (PierreFabre/Aurigene). See, e.g., WO 2012/168944, WO 2015/036927, WO 2015/044900, WO 2015/033303, WO 2013/144704, WO 2013/132317, and WO 2011/161699. In some instances, the PD-1 binding antagonist is a small molecule that inhibits PD-1.

In some instances, the PD-1 binding antagonist is an anti-PD-1 antibody. A variety of anti-PD-1 antibodies can be utilized in the methods and uses disclosed herein. In any of the instances herein, the PD-1 antibody can bind to a human PD-1 or a variant thereof. In some instances, the anti-PD-1 antibody is a monoclonal antibody. In some instances, the anti-PD-1 antibody is an antibody fragment selected from the group consisting of Fab, Fab′, Fab′-SH, Fv, scFv, and (Fab′)₂ fragments. In some instances, the anti-PD-1 antibody is a humanized antibody. In other instances, the anti-PD-1 antibody is a human antibody. Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21.

In some instances, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO 2006/121168.

In some instances, the anti-PD-1 antibody is pembrolizumab (CAS Registry Number: 1374853-91-4). Pembrolizumab (Merck), also known as MK-3475, Merck 3475, lambrolizumab, SCH-900475, and KEYTRUDA®, is an anti-PD-1 antibody described in WO 2009/114335.

In some instances, the anti-PD-1 antibody is MEDI-0680 (AMP-514; AstraZeneca). MEDI-0680 is a humanized IgG4 anti-PD-1 antibody.

In some instances, the anti-PD-1 antibody is PDR001 (CAS Registry No. 1859072-53-9; Novartis). PDR001 is a humanized IgG4 anti-PD-1 antibody that blocks the binding of PD-L1 and PD-L2 to PD-1.

In some instances, the anti-PD-1 antibody is REGN2810 (Regeneron). REGN2810 is a human anti-PD-1 antibody.

In some instances, the anti-PD-1 antibody is BGB-108 (BeiGene).

In some instances, the anti-PD-1 antibody is BGB-A317 (BeiGene).

In some instances, the anti-PD-1 antibody is JS-001 (Shanghai Junshi). JS-001 is a humanized anti-PD-1 antibody.

In some instances, the anti-PD-1 antibody is STI-A1110 (Sorrento). STI-A1110 is a human anti-PD-1 antibody.

In some instances, the anti-PD-1 antibody is INCSHR-1210 (Incyte). INCSHR-1210 is a human IgG4 anti-PD-1 antibody.

In some instances, the anti-PD-1 antibody is PF-06801591 (Pfizer).

In some instances, the anti-PD-1 antibody is TSR-042 (also known as ANB011; Tesaro/AnaptysBio).

In some instances, the anti-PD-1 antibody is AM0001 (ARMO Biosciences).

In some instances, the anti-PD-1 antibody is ENUM 244C8 (Enumeral Biomedical Holdings). ENUM 244C8 is an anti-PD-1 antibody that inhibits PD-1 function without blocking binding of PD-L1 to PD-1.

In some instances, the anti-PD-1 antibody is ENUM 388D4 (Enumeral Biomedical Holdings). ENUM 388D4 is an anti-PD-1 antibody that competitively inhibits binding of PD-L1 to PD-1.

In some instances, the anti-PD-1 antibody comprises the six HVR sequences (e.g., the three heavy chain HVRs and the three light chain HVRs) and/or the heavy chain variable domain and light chain variable domain from an anti-PD-1 antibody described in WO 2015/112800, WO 2015/112805, WO 2015/112900, US20150210769, WO2016/089873, WO 2015/035606, WO 2015/085847, WO 2014/206107, WO 2012/145493, U.S. Pat. No. 9,205,148, WO 2015/119930, WO 2015/119923, WO 2016/032927, WO 2014/179664, WO 2016/106160, and WO 2014/194302.

In a still further specific aspect, the anti-PD-1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-1 antibody is aglycosylated.

### C. PD-L2 Binding Antagonists

In some instances, the PD-1 axis binding antagonist is a PD-L2 binding antagonist. In some instances, the PD-L2 binding antagonist is a molecule that inhibits the binding of PD-L2 to its ligand binding partners. In a specific aspect, the PD-L2 binding ligand partner is PD-1. The PD-L2 binding antagonist may be, without limitation, an antibody, an antigen-binding fragment thereof, an immunoadhesin, a fusion protein, an oligopeptide, or a small molecule.

In some instances, the PD-L2 binding antagonist is an anti-PD-L2 antibody. In any of the instances herein, the anti-PD-L2 antibody can bind to a human PD-L2 or a variant thereof. In some instances, the anti-PD-L2 antibody is a monoclonal antibody. In some instances, the anti-PD-L2 antibody is an antibody fragment selected from the group consisting of Fab, Fab′, Fab′-SH, Fv, scFv, and (Fab′)₂ fragments. In some instances, the anti-PD-L2 antibody is a humanized antibody. In other instances, the anti-PD-L2 antibody is a human antibody. In a still further specific aspect, the anti-PD-L2 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-L2 antibody is aglycosylated.

It is expressly contemplated that such PD-L1 axis binding antagonist antibodies (e.g., anti-PD-L1 antibodies, anti-PD-1 antibodies, and anti-PD-L2 antibodies), or other antibodies described herein for use in any of the instances enumerated above may have any of the features, singly or in combination, described in Sections 1-7 below.

### 1. Antibody Affinity

In certain instances, an antibody described herein (e.g., an anti-PD-L1 antibody) has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

In one instance, Kd is measured by a radiolabeled antigen binding assay (RIA). In one instance, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20@) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another instance, Kd is measured using a BIACORE® surface plasmon resonance assay. For example, an assay using a BIACORE®-2000 or a BIACORE®-3000 (BIAcore, Inc., Piscataway, NJ) is performed at 25° C. with immobilized antigen CM5 chips at ˜10 response units (RU). In one instance, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, for example, Chen et al., J. Mol. Biol. 293:865-881 (1999). If the on-rate exceeds 106 M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

### 2. Antibody Fragments

In certain instances, an antibody (e.g., an anti-PD-L1 antibody) described herein is an antibody fragment. Antibody fragments include, but are not limited to, Fab, Fab′, Fab′-SH, F (ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134 (2003); and Hollinger et al. Proc. Natl. Acad. Sci. USA 90:6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain instances, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

### 3. Chimeric and Humanized Antibodies

In certain instances, an antibody (e.g., an anti-PD-L1 antibody) described herein is a chimeric antibody. Certain chimeric antibodies are described, e.g., in U.S. Pat. No. 4,816,567; and Morrison et al. Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain instances, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some instances, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Natl. Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain instances, an antibody (e.g., an anti-PD-L1 antibody) described herein is a human antibody. Human antibodies can be produced using various techniques known in the art. Human antibodies are described generally in van Dijk and van de Winkel, Curr. Opin. Pharmacol. 5:368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26 (4): 265-268 (2006) (describing human-human hybridomas). Human hybridoma technology (Trioma technology) is also described in Vollmers and Brandlein, Histology and Histopathology, 20 (3): 927-937 (2005) and Vollmers and Brandlein, Methods and Findings in Experimental and Clinical Pharmacology, 27 (3): 185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

Antibodies (e.g., anti-PD-L1 antibodies) may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352:624-628 (1991); Marks et al., J. Mol. Biol. 222:581-597 (1992); Marks and Bradbury, in Methods in Molecular Biology 248:161-175 (Lo, ed., Human Press, Totowa, NJ, 2003); Sidhu et al., J. Mol. Biol. 338 (2): 299-310 (2004); Lee et al., J. Mol. Biol. 340 (5): 1073-1093 (2004); Fellouse, Proc. Natl. Acad. Sci. USA 101 (34): 12467-12472 (2004); and Lee et al., J. Immunol. Methods 284 (1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12:433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12:725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227:381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 6. Multispecific Antibodies

In any one of the above aspects, an antibody (e.g., an anti-PD-L1 antibody) described herein may be a multispecific antibody, for example, a bispecific antibody. Multispecific antibodies are monoclonal antibodies that have binding specificities for at least two different sites. In certain instances, an antibody described herein is a multispecific antibody, e.g., a bispecific antibody. In certain instances, one of the binding specificities is for PD-L1 and the other is for any other antigen. In certain instances, bispecific antibodies may bind to two different epitopes of PD-L1. Bispecific antibodies may also be used to localize cytotoxic agents to cells which express PD-L1. Bispecific antibodies can be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305:537 (1983)), WO 93/08829, and Traunecker et al., EMBO J. 10:3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (see, e.g., WO 2009/089004A1); cross-linking two or more antibodies or fragments (see, e.g., U.S. Pat. No. 4,676,980, and Brennan et al., Science 229:81 (1985)); using leucine zippers to produce bi-specific antibodies (see, e.g., Kostelny et al., J. Immunol. 148 (5): 1547-1553 (1992)); using “diabody” technology for making bispecific antibody fragments (see, e.g., Hollinger et al., Proc. Natl. Acad. Sci. USA 90:6444-6448 (1993)); using single-chain Fv (sFv) dimers (see, e.g., Gruber et al., J. Immunol. 152:5368 (1994)); and preparing trispecific antibodies as described, e.g., in Tutt et al. J. Immunol. 147:60 (1991).

Engineered antibodies with three or more functional antigen binding sites, including “Octopus antibodies,” are also included herein (see, e.g., US 2006/0025576A1).

An antibody or antigen-binding fragment thereof described herein also includes a “Dual Acting FAb” or “DAF” comprising an antigen-binding site that binds to PD-L1 as well as another, different antigen.

### 7. Antibody Variants

In certain instances, amino acid sequence variants of the antibodies described herein (e.g., anti-PD-L1 antibodies) are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

### I. Substitution, Insertion, and Deletion Variants

In certain instances, antibody variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved Antibody-Dependent Cell-Mediated Cytotoxicity (ADCC) or Complement Dependent Cytotoxicity (CDC).

TABLE 1
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity and/or reduced immunogenicity) relative to the parent antibody and/or will have substantially retained certain biological properties of the parent antibody. An exemplary substitutional variant is an affinity matured antibody, which may be conveniently generated, for example, using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some instances of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain instances, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen-contacting residues in the HVRs. In certain instances of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

II. Glycosylation variants

In certain instances, antibodies described herein can be altered to increase or decrease the extent to which the antibody is glycosylated. Addition or deletion of glycosylation sites to an antibody of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some instances, modifications of the oligosaccharide in an antibody of the invention may be made in order to create antibody variants with certain improved properties.

In one instance, antibody variants may have a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, for example, U.S. Patent Publication Nos. US 2003/0157108 and US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Lec13 CHO cells deficient in protein fucosylation (Ripka et al. Arch. Biochem. Biophys. 249:533-545 (1986); U.S. Pat. Appl. No. US 2003/0157108 A1; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87:614 (2004); Kanda, Y. et al., Biotechnol. Bioeng., 94 (4): 680-688 (2006); and WO2003/085107).

Antibody variants may also have bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### III. Fc Region Variants

In certain instances, one or more amino acid modifications may be introduced into the Fc region of an antibody of the invention, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain instances, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest are described in U.S. Pat. No. 5,500,362 (see, e.g., Hellstrom, I. et al. Proc. Natl. Acad. Sci. USA 83:7059-7063 (1986)) and Hellstrom, I et al., Proc. Natl. Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CYTOTOX 96® non-radioactive cytotoxicity assay (Promega, Madison, WI))). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Natl. Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, e.g., Gazzano-Santoro et al., J. Immunol. Methods 202:163 (1996); Cragg et al., Blood. 101:1045-1052 (2003); and Cragg et al., Blood. 103:2738-2743 (2004)). FcRn binding and in vivo clearance/half-life determinations can also be performed using methods known in the art (see, e.g., Petkova et al. Int'l. Immunol. 18 (12): 1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

Certain antibody variants with improved or diminished binding to FcRs are described. (See, e.g., U.S. Pat. No. 6,737,056; WO 2004/056312, and Shields et al., J. Biol. Chem. 9 (2): 6591-6604 (2001).)

In certain instances, an antibody variant comprises an Fc region with one or more amino acid substitutions which improve ADCC, e.g., substitutions at positions 298, 333, and/or 334 of the Fc region (EU numbering of residues).

In some instances, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### IV. Cysteine Engineered Antibody Variants

In certain instances, it may be desirable to create cysteine engineered antibodies, e.g., “thioMAbs,” in which one or more residues of an antibody are substituted with cysteine residues. In particular instances, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain instances, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### V. Antibody Derivatives

In certain instances, an antibody described herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another instance, conjugates of an antibody and nonproteinaceous moiety that may be selectively heated by exposure to radiation are provided. In one instance, the nonproteinaceous moiety is a carbon nanotube (Kam et al., Proc. Natl. Acad. Sci. USA 102:11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### VI. Immunoconjugates

The invention also provides immunoconjugates comprising an antibody described herein (e.g., an anti-PD-L1 antibody) conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one instance, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another instance, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another instance, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron. Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate) which are commercially available (e.g., from Pierce Biotechnology, Inc., Rockford, IL., U.S.A).

### VI. Platinum-Based Chemotherapies

Provided herein are methods for treating or delaying progression of bladder cancer (e.g., UC, including locally advanced or metastatic UC) in a subject comprising administering to the subject a treatment regimen comprising an effective amount of a PD-1 axis binding antagonist (e.g., an anti-PD-L1 antibody (e.g., atezolizumab)) and a platinum-based chemotherapy (e.g., cisplatin or carboplatin and gemcitabine). Further provided herein are methods of enhancing immune function in a subject having a bladder cancer (e.g., UC, including locally advanced or metastatic UC) comprising administering to the subject a treatment regimen comprising an effective amount of a PD-1 axis binding antagonist (e.g., an anti-PD-L1 antibody (e.g., atezolizumab)) and a platinum-based chemotherapy (e.g., cisplatin or carboplatin and gemcitabine). Also provided are related compositions (e.g., pharmaceutical compositions) for use, kits, and articles of manufacture. Any of the methods, compositions for use, kits, or articles of manufacture described herein may include or involve any of the platinum-based chemotherapies described below.

Any suitable platinum-based chemotherapy may be used. In some embodiments, the platinum-based chemotherapy includes a platinum-based chemotherapeutic agent (e.g., cisplatin, carboplatin, oxaliplatin, nedaplatin, triplatin tetranitrate, phenanthriplatin, picoplatin, lipoplatin, or satraplatin). In some embodiments, the platinum-based chemotherapy includes cisplatin. In some embodiments, the platinum-based chemotherapy includes carboplatin.

In some embodiments, the platinum-based chemotherapy further includes one or more additional chemotherapeutic agents. For example, a platinum-based chemotherapy may further include any of the chemotherapeutic agents described herein. In one example, the platinum-based chemotherapy further includes a nucleoside analog. Any suitable nucleoside analog may be used, e.g., gemcitabine, cytarabine, fludarabine, or cladribine. In some embodiments, the nucleoside analog is gemcitabine. For example, in some embodiments, the platinum-based chemotherapy includes cisplatin and gemcitabine. In other embodiments, the platinum-based chemotherapy includes carboplatin and gemcitabine.

In another example, the platinum-based chemotherapy may include cisplatin, methotrexate, vinblastine, and doxorubicin, which is also known in the art as MVAC.

In another example, the platinum-based chemotherapy may include dose-dense methotrexate, vinblastine, doxorubicin, and cisplatin, which is also known in the art as DDMVAC.

In another example, the platinum-based chemotherapy may include cisplatin and fluorouracil (5-FU).

In another example, the platinum-based chemotherapy may include cisplatin, methotrexate, and vinblastine, which is also known in the art as CMV.

In another example, the platinum-based chemotherapy may include methotrexate, carboplatin, and vincristine, which is also known in the art as M-CAVI.

In another example, the platinum-based chemotherapy may include cisplatin and a taxane (e.g., paclitaxel).

### VII. Pharmaceutical Compositions and Formulations

Also provided herein are pharmaceutical compositions and formulations comprising a PD-1 axis binding antagonist and/or an antibody described herein (such as an anti-PD-L1 antibody (e.g., atezolizumab)) and, optionally, a pharmaceutically acceptable carrier. The invention also provides pharmaceutical compositions and formulations comprising one or more members of a platinum-based chemotherapy (e.g., cisplatin or carboplatin and gemcitabine), and optionally, a pharmaceutically acceptable carrier.

Pharmaceutical compositions and formulations as described herein can be prepared by mixing the active ingredients (e.g., a PD-1 axis binding antagonist (e.g., an anti-PD-L1 antibody (e.g., atezolizumab) and/or a platinum-based chemotherapy (e.g., cisplatin or carboplatin and gemcitabine) having the desired degree of purity with one or more optional pharmaceutically acceptable carriers (see, e.g., Remington's Pharmaceutical Sciences 16th edition, Osol, A. Ed. (1980)), e.g., in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The compositions and formulations herein may also contain more than one active ingredient as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g., films or microcapsules. The formulations to be used for in vivo administration are generally sterile. Sterility may be readily accomplished, e.g., by filtration through sterile filtration membranes.

### VIII. Articles of Manufacture or Kits

Provided herein are kits and articles of manufacture, which may be used, for example, for carrying out the methods and assays disclosed herein.

For example, provided herein is a kit comprising: (a) one or more reagents for an immune-directed PD-L1 assay; and (b) one or more reagents for an immune-agnostic PD-L1 assay.

The immune-directed PD-L1 assay or the immune-agnostic PD-L1 assay may be an AHC assay (e.g., an IHC assay).

The kit may include one or more anti-PD-L1 diagnostic antibodies. Any suitable anti-PD-L1 diagnostic antibodies may be included, e.g., SP142 (Ventana), SP263 (Ventana), 22C3 (Dako), 28-8 (Dako), E1L3N (Cell Signaling Technology), 4059 (ProSci, Inc.), h5H1 (Advanced Cell Diagnostics), and 9A11. In some examples, the anti-PD-L1 diagnostic antibody is SP142. In other examples, the anti-PD-L1 diagnostic antibody is SP263, 22C3, or 28-8.

For example, provided herein is a kit comprising: (a) the VENTANA SP142 anti-PD-L1 diagnostic antibody; and (b) the Dako 22C3 anti-PD-L1 diagnostic antibody, the VENTANA SP263 anti-PD-L1 diagnostic antibody, or the 28-8 anti-PD-L1 diagnostic antibody.

In some examples, the kit further comprises one or more reagents for visualizing the anti-PD-L1 diagnostic antibodies of (a) and (b), e.g., one or more secondary antibodies, one or more chromogens, one or more amplification reagents (e.g., one or more tyramide amplification reagents), one or more buffers, and the like.

In another aspect, provided herein is a kit for identifying a tumor likely to respond to a PD-1 axis binding antagonist, the kit comprising: (a) reagents for staining a first portion of the tumor with an immune-directed PD-L1 assay to obtain a first stained sample; (b) reagents for staining a second portion of the tumor with an immune-agnostic PD-L1 assay to obtain a second stained sample; and optionally: (c) instructions to generate a first score by applying a first scoring algorithm to the first stained sample; (d) instructions to generate a second score by applying a second scoring algorithm the second stained sample; and (e) instructions to compare the first score to a first cutoff and the second score to a second cutoff, wherein the tumor is likely to respond to the PD-1 axis binding antagonist when both the first score meets or exceeds the first cutoff and the second score meets or exceeds the second cutoff.

In some aspects, the immune-directed PD-L1 assay has: (i) at least an 80% overall percent agreement (OPA) with an SP142 Assay using the first scoring algorithm at the first cutoff value; (ii) at least an 80% positive percent agreement (PPA) with an SP142 Assay using the first scoring algorithm at the first cutoff value; (iii) at least an 80% negative percent agreement (NPA) with an SP142 Assay using the first scoring algorithm at the first cutoff value; (iv) at least an 80% PPA and at least an 80% NPA with an SP142 Assay using the first scoring algorithm at the first cutoff value; (v) at least an 80% PPA and at least an 80% OPA with an SP142 Assay using the first scoring algorithm at the first cutoff value; (vi) at least an 80% NPA and at least an 80% OPA with an SP142 Assay using the first scoring algorithm at the first cutoff value; and/or (vii) at least an 80% OPA, at least an 80% PPA, and at least an 80% NPA with an SP142 Assay using the first scoring algorithm at the first cutoff value.

In some aspects, the immune-agnostic PD-L1 assay has: (i) at least an 80% overall percent agreement (OPA) with an 22C3 Assay using the second scoring algorithm at the second cutoff value; (ii) at least an 80% positive percent agreement (PPA) with an 22C3 Assay using the second scoring algorithm at the second cutoff value; (iii) at least an 80% negative percent agreement (NPA) with an 22C3 Assay using the second scoring algorithm at the second cutoff value; (iv) at least an 80% PPA and at least an 80% NPA with an 22C3 Assay using the second scoring algorithm at the second cutoff value; (v) at least an 80% PPA and at least an 80% OPA with an 22C3 Assay using the second scoring algorithm at the second cutoff value; (vi) at least an 80% NPA and at least an 80% OPA with an 22C3 Assay using the second scoring algorithm at the second cutoff value; and/or (vii) at least an 80% OPA, at least an 80% PPA, and at least an 80% NPA with an 22C3 Assay using the second scoring algorithm at the second cutoff value.

In another aspect, an article of manufacture or a kit is provided comprising a PD-1 axis binding antagonist (e.g., an anti-PD-L1 antibody (e.g., atezolizumab)) and/or one or more additional therapeutic agents (e.g., a platinum-based chemotherapy (e.g., cisplatin or carboplatin and gemcitabine)). In some embodiments, the article of manufacture or kit further comprises package insert comprising instructions for using the PD-1 axis binding antagonist to treat or delay progression of a bladder cancer (e.g., UC, including locally advanced or metastatic UC) in a subject or to enhance immune function of a subject having a bladder cancer (e.g., UC, including locally advanced or metastatic UC). In some embodiments, the article of manufacture or kit further comprises package insert comprising instructions for using the PD-1 axis binding antagonist in combination with a platinum-based chemotherapy (e.g., cisplatin or carboplatin and gemcitabine) to treat or delay progression of bladder cancer (e.g., UC, including locally advanced or metastatic UC) in a subject or to enhance immune function of a subject having a bladder cancer (e.g., UC, including locally advanced or metastatic UC). Any of the PD-1 axis binding antagonists and/or platinum-based chemotherapies described herein may be included in the article of manufacture or kits.

In some embodiments, the PD-1 axis binding antagonist (e.g., an anti-PD-L1 antibody (e.g., atezolizumab)) and the platinum-based chemotherapy (e.g., cisplatin or carboplatin and gemcitabine) are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., an additional chemotherapeutic agent or anti-neoplastic agent). Suitable containers for the one or more agent include, for example, bottles, vials, bags and syringes.

In any of the preceding kits, the anti-PD-L1 antibody may be atezolizumab. The kit may include instructions to administer atezolizumab to the subject at any suitable dosage. In some embodiments, the kit includes instructions to administer atezolizumab to the subject intravenously at a dose of about 840 mg every 2 weeks, about 1200 mg every 3 weeks, or about 1680 mg every 4 weeks. In some embodiments, the kit includes instructions to administer atezolizumab to the subject intravenously at a dose of about 1200 mg every 3 weeks. In some embodiments, the kit includes instructions to administer atezolizumab to the subject in a 21-day dosing cycle. In some embodiments, the kit includes instructions to administer atezolizumab to the subject intravenously at a dose of about 1200 mg on Day-2 to Day 4 of each 21-day dosing cycle. In some embodiments, the kit includes instructions to administer atezolizumab to the subject intravenously at a dose of about 1200 mg on Day 1 of each 21-day dosing cycle.

The specification is considered to be sufficient to enable one skilled in the art to practice the invention. Various modifications of the invention in addition to those shown and described herein will become apparent to those skilled in the art from the foregoing description and fall within the scope of the appended claims.

### EMBODIMENTS

1. A method of identifying a tumor likely to respond to a PD-1 axis binding antagonist, the method comprising:
(a) staining a first portion of the tumor with an immune-directed PD-L1 assay to obtain a first stained sample;
(b) generating a first score by applying a first scoring algorithm to the first stained sample;
(c) staining a second portion of the tumor with an immune-agnostic PD-L1 assay to obtain a second stained sample;
(d) generating a second score by applying a second scoring algorithm the second stained sample; and
(e) comparing the first score to a first cutoff and the second score to a second cutoff, wherein the tumor is likely to respond to the PD-1 axis binding antagonist when both the first score meets or exceeds the first cutoff and the second score meets or exceeds the second cutoff.

2. The method of embodiment 1, wherein the tumor is in a patient, and the method further comprises administering the treatment regimen comprising the PD-1 axis binding antagonist to the patient.

3. A method of treating a cancer in a patient, the method comprising administering to the patient a treatment regimen comprising a PD-1 axis binding antagonist, wherein a tumor sample obtained from the patient has been determined to have:
(i) a first score obtained from applying a first scoring algorithm to a first stained portion of the tumor sample that meets or exceeds a first cutoff, wherein the first stained portion of the tumor sample was stained with an immune-directed PD-L1 assay; and
(ii) a second score obtained from applying a second scoring algorithm to a second stained portion of the tumor sample that meets or exceeds a second cutoff, wherein the second stained portion of the tumor sample was stained with an immune-agnostic PD-L1 assay.

4. The method of any one of embodiments 1-3, wherein the immune-directed PD-L1 assay has:
(i) at least an 80%, at least an 85%, at least a 90%, or at least a 95% overall percent agreement (OPA) with an SP142 Assay using the first scoring algorithm at the first cutoff value;
(ii) at least an 80%, at least an 85%, at least a 90%, or at least a 95% positive percent agreement (PPA) with an SP142 Assay using the first scoring algorithm at the first cutoff value;
(iii) at least an 80%, at least an 85%, at least a 90%, or at least a 95% negative percent agreement (NPA) with an SP142 Assay using the first scoring algorithm at the first cutoff value;
(iv) at least an 80%, at least an 85%, at least a 90%, or at least a 95% PPA and at least an 80%, at least an 85%, at least a 90%, or at least a 95% NPA with an SP142 Assay using the first scoring algorithm at the first cutoff value;
(v) at least an 80%, at least an 85%, at least a 90%, or at least a 95% PPA and at least an 80%, at least an 85%, at least a 90%, or at least a 95% OPA with an SP142 Assay using the first scoring algorithm at the first cutoff value;
(vi) at least an 80%, at least an 85%, at least a 90%, or at least a 95% NPA and at least an 80%, at least an 85%, at least a 90%, or at least a 95% OPA with an SP142 Assay using the first scoring algorithm at the first cutoff value; and/or
(vii) at least an 80%, at least an 85%, at least a 90%, or at least a 95% OPA, at least an 80%, at least an 85%, at least a 90%, or at least a 95% PPA, and at least an 80%, at least an 85%, at least a 90%, or at least a 95% NPA with an SP142 Assay using the first scoring algorithm at the first cutoff value.

5. The method of any one of embodiments 1-4, wherein the immune-agnostic PD-L1 assay has:
(i) at least an 80%, at least an 85%, at least a 90%, or at least a 95% OPA with an 22C3 Assay using the second scoring algorithm at the second cutoff value;
(ii) at least an 80%, at least an 85%, at least a 90%, or at least a 95% PPA with an 22C3 Assay using the second scoring algorithm at the second cutoff value;
(iii) at least an 80%, at least an 85%, at least a 90%, or at least a 95% NPA with an 22C3 Assay using the second scoring algorithm at the second cutoff value;
(iv) at least an 80%, at least an 85%, at least a 90%, or at least a 95% PPA and at least an 80%, at least an 85%, at least a 90%, or at least a 95% NPA with an 22C3 Assay using the second scoring algorithm at the second cutoff value;
(v) at least an 80%, at least an 85%, at least a 90%, or at least a 95% PPA and at least an 80%, at least an 85%, at least a 90%, or at least a 95% OPA with an 22C3 Assay using the second scoring algorithm at the second cutoff value;
(vi) at least an 80%, at least an 85%, at least a 90%, or at least a 95% NPA and at least an 80%, at least an 85%, at least a 90%, or at least a 95% OPA with an 22C3 Assay using the second scoring algorithm at the second cutoff value; and/or
(vii) at least an 80%, at least an 85%, at least a 90%, or at least a 95% OPA, at least an 80%, at least an 85%, at least a 90%, or at least a 95% PPA, and at least an 80%, at least an 85%, at least a 90%, or at least a 95% NPA with an 22C3 Assay using the second scoring algorithm at the second cutoff value.

6. A method of stratifying a tumor having a score with an immune-agnostic PD-L1 assay that exceeds a pre-determined cutoff, the method comprising:
(a) staining a portion of the tumor with an immune-directed PD-L1 assay to obtain a stained sample;
(b) generating an immune-directed PD-L1 assay score by applying a scoring algorithm to the stained sample; and
(c) comparing the immune-directed PD-L1 assay score to a first cutoff, wherein the tumor is likely to respond to a PD-1 axis binding antagonist when the immune-directed PD-L1 assay score meets or exceeds the first cutoff.

7. A method of stratifying a Combined Positive Score (CPS) ≥10% tumor as determined by a 22C3 assay, the method comprising:
(a) staining a portion of the tumor with an immune-directed PD-L1 assay to obtain a stained sample;
(b) generating an immune-directed PD-L1 assay score by applying a scoring algorithm to the stained sample; and
(c) comparing the immune-directed PD-L1 assay score to a first cutoff, wherein the tumor is likely to respond to the PD-1 axis binding antagonist when the immune-directed PD-L1 assay score meets or exceeds the first cutoff.

8. A method of stratifying a CPS ≥10% tumor as determined by an SP263 assay, the method comprising:
(a) staining a portion of the tumor with an immune-directed PD-L1 assay to obtain a stained sample;
(b) generating an immune-directed PD-L1 assay score by applying a scoring algorithm to the stained sample; and
(c) comparing the immune-directed PD-L1 assay score to a first cutoff, wherein the tumor is likely to respond to the PD-1 axis binding antagonist when the immune-directed PD-L1 assay score meets or exceeds the first cutoff.

9. A method of stratifying a CPS ≥10% tumor as determined by a 28-8 assay, the method comprising:
(a) staining a portion of the tumor with an immune-directed PD-L1 assay to obtain a stained sample;
(b) generating an immune-directed PD-L1 assay score by applying a scoring algorithm to the stained sample; and
(c) comparing the immune-directed PD-L1 assay score to a first cutoff, wherein the tumor is likely to respond to the PD-1 axis binding antagonist when both the immune-directed PD-L1 assay score meets or exceeds the first cutoff.

10. The method of any one of embodiments 1-9, wherein the tumor is a bladder cancer, a kidney cancer, a lung cancer, a cancer of the urinary tract, a breast cancer, a prostate cancer, a cancer of the peritoneum, a hepatocellular cancer, a gastric or stomach cancer, a pancreatic cancer, a glioblastoma, a cervical cancer, an ovarian cancer, a liver cancer, a hepatoma, a colon cancer, a rectal cancer, a colorectal cancer, an endometrial or uterine carcinoma, a salivary gland carcinoma, a prostate cancer, a vulval cancer, a thyroid cancer, a hepatic carcinoma, an anal carcinoma, a penile carcinoma, a melanoma, a multiple myeloma or B-cell lymphoma, a chronic lymphocytic leukemia (CLL), an acute lymphoblastic leukemia (ALL), an acute myologenous leukemia (AML), a hairy cell leukemia, a chronic myeloblastic leukemia (CML), a post-transplant lymphoproliferative disorder (PTLD), a myelodysplastic syndrome (MDS), Meigs' syndrome, a brain cancer, or a head and neck cancer.

11. The method of embodiment 10, wherein the tumor is a bladder cancer.

12. The method of embodiment 11, wherein the bladder cancer is a urothelial carcinoma (UC).

13. The method of embodiment 12, wherein the UC is a locally advanced or metastatic UC.

14. The method of any one of embodiments 1-13, wherein the immune-directed PD-L1 assay comprises a PD-L1 immunohistochemical (IHC) assay comprising the VENTANA SP142 anti-PD-L1 diagnostic antibody.

15. The method of any one of embodiments 1-6 and 14, wherein the immune-agnostic PD-L1 assay comprises a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody.

16. The method of any one of embodiments 1-15, wherein the first cutoff is a detectable expression level of PD-L1 in tumor-infiltrating immune cells that comprise 5% or more of the tumor sample.

17. The method of any one of embodiments 1-5 and 10⁻¹⁶, wherein the second cutoff is a CPS ≥10%.

18. A method of labeling PD-L1 in a tumor sample, the method comprising the following steps:
(a) contacting the tumor sample with the VENTANA SP142 anti-PD-L1 diagnostic antibody;
(b) contacting the tumor sample with the Dako 22C3 anti-PD-L1 diagnostic antibody, the VENTANA SP263 anti-PD-L1 diagnostic antibody, or the 28-8 anti-PD-L1 diagnostic antibody; and
(c) visualizing the anti-PD-L1 diagnostic antibodies of steps (a) and (b) with one or more detectable reagents that generates a detectable signal for both of the anti-PD-L1 diagnostic antibodies.

19. The method of embodiment 18, wherein the detectable signal for the VENTANA SP142 anti-PD-L1 diagnostic antibody is an amplified signal.

20. The method of embodiment 19, wherein the amplified signal is generated by tyramide signal amplification.

21. The method of any one of embodiment 18-20, wherein steps (a) and (b) are performed simultaneously.

22. The method of any one of embodiment 18-20, wherein steps (a) and (b) are performed sequentially.

23. The method of any one of embodiments 18-22, wherein steps (a) and (b) are performed in different sections of the tumor sample or in the same section of the tumor sample.

24. The method of embodiment 23, wherein the different sections of the tumor sample are consecutive sections.

25. The method of any one of embodiments 18-24, wherein the visualizing comprises IHC or immunofluorescence (IF).

26. The method of embodiment 25, wherein the visualizing comprises IHC.

27. The method of any one of embodiments 18-26, wherein the tumor sample is obtained from a patient having a cancer.

28. The method of any one of embodiments 1, 2, and 4-17, wherein the tumor is a tumor sample obtained from a patient having a cancer.

29. The method of any one of embodiments 3-5 and 18-28, wherein the tumor sample is an FFPE tumor sample, an archival tumor sample, a fresh tumor sample, or a frozen tumor sample.

30. The method of any one of embodiments 3-5 and 27-29, wherein the cancer is a bladder cancer, a kidney cancer, a lung cancer, a cancer of the urinary tract, a breast cancer, a prostate cancer, a cancer of the peritoneum, a hepatocellular cancer, a gastric or stomach cancer, a pancreatic cancer, a glioblastoma, a cervical cancer, an ovarian cancer, a liver cancer, a hepatoma, a colon cancer, a rectal cancer, a colorectal cancer, an endometrial or uterine carcinoma, a salivary gland carcinoma, a prostate cancer, a vulval cancer, a thyroid cancer, a hepatic carcinoma, an anal carcinoma, a penile carcinoma, a melanoma, a multiple myeloma or B-cell lymphoma, a chronic lymphocytic leukemia (CLL), an acute lymphoblastic leukemia (ALL), an acute myologenous leukemia (AML), a hairy cell leukemia, a chronic myeloblastic leukemia (CML), a post-transplant lymphoproliferative disorder (PTLD), a myelodysplastic syndrome (MDS), Meigs' syndrome, a brain cancer, or a head and neck cancer.

31. The method of embodiment 30, wherein the cancer is a bladder cancer.

32. The method of embodiment 31, wherein the bladder cancer is a urothelial carcinoma (UC).

33. The method of embodiment 32, wherein the UC is a locally advanced or metastatic UC.

34. The method of embodiment 33, wherein the patient is previously untreated for the locally advanced or metastatic UC.

35. The method of embodiment 33 or 34, wherein the locally advanced or metastatic UC is histologically documented, locally advanced (T4b, any N; or any T, N2-3) or metastatic urothelial carcinoma (mUC) (M1, Stage IV).

36. The method of any one of embodiments 33-35, wherein the cancer is locally advanced UC.

37. The method of any one of embodiments 13 and 33-36, wherein the locally advanced UC is inoperable.

38. The method of any one of embodiments 33-35, wherein the cancer is metastatic UC.

39. The method of any one of embodiments 33-38, wherein the patient is eligible for treatment with a platinum-based chemotherapy.

40. The method of embodiment 39, wherein the patient is eligible for treatment with a cisplatin-based chemotherapy.

41. The method of embodiments 33-40, wherein the patient has not received prior chemotherapy for the locally advanced or metastatic UC.

42. The method of any one of embodiments 33-40, wherein the patient has previously received an adjuvant or neoadjuvant chemotherapy or chemoradiation for UC, and has had a treatment-free interval of more than 12 months between the last administration of the adjuvant or neoadjuvant chemotherapy or chemoradiation and the date of recurrence.

43. The method of any one of embodiments 1, 2, and 4-42, wherein the method identifies the patient as one who may benefit from a treatment regimen comprising a PD-1 axis binding antagonist.

44. The method of any one of embodiments 4-43, further comprising administering the treatment regimen comprising a PD-1 axis binding antagonist to the patient.

45. The method of any one of embodiments 1-5, 43, and 44, wherein the PD-1 axis binding antagonist is a PD-L1 binding antagonist, a PD-1 binding antagonist, or a PD-L2 binding antagonist.

46. The method of embodiment 45, wherein the PD-1 axis binding antagonist is a PD-L1 binding antagonist.

47. The method of embodiment 46, wherein the PD-1 axis binding antagonist is an anti-PD-L1 antibody.

48. The method of embodiment 47, wherein the anti-PD-L1 antibody comprises the following HVRs:

(i) an HVR-H1 sequence of
(SEQ ID NO: 3)
GFTFSDSWIH;
(ii) an HVR-H2 sequence of
(SEQ ID NO: 4)
AWISPYGGSTYYADSVKG;
(iii) an HVR-H3 sequence of
(SEQ ID NO: 5)
RHWPGGFDY;
(iv) an HVR-L1 sequence of
(SEQ ID NO: 6)
RASQDVSTAVA;
(v) an HVR-L2 sequence of
(SEQ ID NO: 7)
SASFLYS;
and
(vi) an HVR-L3 sequence of
(SEQ ID NO: 8)
QQYLYHPAT.

49. The method of embodiment 47 or 48, wherein the anti-PD-L1 antibody comprises:
(a) a VH domain comprising the amino acid sequence of SEQ ID NO: 9; and
(b) a VL domain comprising the amino acid sequence of SEQ ID NO: 10.

50. The method of any one of embodiments 47-49, wherein the anti-PD-L1 antibody is atezolizumab.

51. The method of embodiment 50, wherein atezolizumab is administered to the patient intravenously at a dose of about 840 mg every 2 weeks, about 1200 mg every 3 weeks, or about 1680 mg every 4 weeks.

52. The method of embodiment 51, wherein atezolizumab is administered to the patient intravenously at a dose of about 1200 mg every 3 weeks.

53. The method of embodiment 52, wherein atezolizumab is administered to the patient in 21-day dosing cycles, and wherein atezolizumab is administered to the patient intravenously at a dose of about 1200 mg on Day-2 to Day 4 of each 21-day dosing cycle.

54. The method of embodiment 53, wherein atezolizumab is administered to the patient intravenously at a dose of about 1200 mg on Day 1 of each 21-day dosing cycle.

55. The method of any one of embodiments 1-5 and 43-54, wherein the PD-1 axis binding antagonist is administered to the patient as a monotherapy.

56. The method of any one of embodiments 2-5 and 43-54, wherein the PD-1 axis binding antagonist is administered to the patient in combination with one or more additional therapeutic agents.

57. The method of embodiment 56, wherein the one or more additional therapeutic agents comprise a platinum-based chemotherapy.

58. The method of embodiment 57, wherein the platinum-based chemotherapy comprises a platinum-based chemotherapeutic agent and a nucleoside analog.

59. The method of embodiment 58, wherein the platinum-based chemotherapeutic agent is cisplatin, carboplatin, or oxaliplatin.

60. The method of embodiment 59, wherein the platinum-based chemotherapeutic agent is cisplatin.

61. The method of embodiment 59, wherein the platinum-based chemotherapeutic agent is carboplatin.

62. The method of any one of embodiments 58-61, wherein the nucleoside analog is gemcitabine.

63. The method of any one of embodiments 58-62, wherein the platinum-based chemotherapy comprises cisplatin and gemcitabine or carboplatin and gemcitabine.

64. The method of embodiment 63, wherein the platinum-based chemotherapy comprises cisplatin and gemcitabine.

65. The method of embodiment 63, wherein the platinum-based chemotherapy comprises carboplatin and gemcitabine.

66. The method of any one of embodiments 43-65, wherein the benefit from the treatment regimen comprising the PD-1 axis binding antagonist is in terms of overall survival (OS).

67. The method of embodiment 66, wherein the treatment regimen extends the patient's OS by from about 5.7 months to about 17 months as compared to treatment with a platinum-based chemotherapy without the PD-1 axis binding antagonist.

68. The method of embodiment 67, wherein the treatment regimen extends the patient's OS by about 11.3 months as compared to treatment with a platinum-based chemotherapy without the PD-1 axis binding antagonist.

69. The method of any one of embodiments 2-6 and 27-68, wherein the patient is a human.

70. The method of any one of embodiments 3-5 and 16-69, wherein the tumor sample obtained from the patient has the presence of discernible PD-L1 staining of any intensity in tumor-infiltrating immune cells covering >5% of tumor area occupied by tumor cells, associated intratumoral, and contiguous peritumoral stroma, as determined by the PD-L1 IHC assay comprising the VENTANA SP142 anti-PD-L1 diagnostic antibody.

71. The method of any one of embodiments 3-5 and 16-69, wherein the tumor sample obtained from the patient has a CPS of ≥10 using a PD-L1 IHC assay comprising the Dako 22C3 anti-PD-L1 diagnostic antibody.

72. The method of any one of embodiments 3-5 and 16-69, wherein the tumor sample obtained from the patient has a CPS of ≥10 using a PD-L1 IHC assay comprising the VENTANA SP263 anti-PD-L1 diagnostic antibody.

73. The method of any one of embodiments 3-5 and 16-69, wherein the tumor sample obtained from the patient has a CPS of ≥10 using a PD-L1 IHC assay comprising the 28-8 anti-PD-L1 diagnostic antibody.

74. An assay for determining the presence or expression level of PD-L1 in a tumor sample obtained from a patient suffering from a cancer, the assay comprising:
(a) determining the presence or expression level of PD-L1 in a tumor sample obtained from the patient using a PD-L1 IHC assay comprising the VENTANA SP142 anti-PD-L1 diagnostic antibody; and
(b) determining the presence or expression level of PD-L1 in the tumor sample obtained from the patient using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody.

75. The assay of embodiment 74, wherein the tumor sample obtained from the patient has a detectable expression level of PD-L1 in tumor-infiltrating immune cells that comprise 5% or more of the tumor sample using a PD-L1 IHC assay comprising the VENTANA SP142 anti-PD-L1 diagnostic antibody.

76. The assay of embodiment 74 or 75, wherein the tumor sample obtained from the patient has the presence of discernible PD-L1 staining of any intensity in tumor-infiltrating immune cells covering ≥5% of tumor area occupied by tumor cells, associated intratumoral, and contiguous peritumoral stroma, as determined by the PD-L1 IHC assay comprising the VENTANA SP142 anti-PD-L1 diagnostic antibody.

77. The assay of any one of embodiments 74-76, wherein the tumor sample obtained from the patient has a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody.

78. The assay of any one of embodiments 74-77, wherein the tumor sample obtained from the patient has:
a detectable expression level of PD-L1 in tumor-infiltrating immune cells that comprise 5% or more of the tumor sample using the PD-L1 IHC assay comprising the VENTANA SP142 anti-PD-L1 diagnostic antibody; and
a CPS of ≥10 using the PD-L1 IHC assay comprising the Dako 22C3 anti-PD-L1 diagnostic antibody.

79. The assay of any one of embodiment 74-78, wherein steps (a) and (b) are performed simultaneously.

80. The assay of any one of embodiment 74-79, wherein steps (a) and (b) are performed sequentially.

81. The assay of any one of embodiments 74-80, wherein steps (a) and (b) are performed in different sections of the tumor sample or in the same section of the tumor sample.

82. The assay of embodiment 81, wherein the different sections of the tumor sample are consecutive sections.

83. The assay of any one of embodiments 74-82, wherein the cancer is locally advanced or metastatic urothelial carcinoma.

84. The assay of embodiment 83, wherein the patient is previously untreated for the locally advanced or metastatic urothelial carcinoma.

85. The assay of any one of embodiments 74-84, wherein the assay is used for (i) selecting a therapy for treating a locally advanced or metastatic UC in a patient in need thereof or (ii) identifying a patient having a locally advanced or metastatic UC who may benefit from a treatment regimen comprising an anti-PD-L1 antibody.

86. The assay of embodiment 85, wherein the anti-PD-L1 antibody comprises the following HVRs:

(i) an HVR-H1 sequence of
(SEQ ID NO: 3)
GFTFSDSWIH;
(ii) an HVR-H2 sequence of
(SEQ ID NO: 4)
AWISPYGGSTYYADSVKG;
(iii) an HVR-H3 sequence of
(SEQ ID NO: 5)
RHWPGGFDY;
(iv) an HVR-L1 sequence of
(SEQ ID NO: 6)
RASQDVSTAVA;
(v) an HVR-L2 sequence of
(SEQ ID NO: 7)
SASFLYS;
and
(vi) an HVR-L3 sequence of
(SEQ ID NO: 8)
QQYLYHPAT.

87. The assay of any one of embodiments 74-86, wherein the tumor sample is an FFPE tumor sample, an archival tumor sample, a fresh tumor sample, or a frozen tumor sample.

88. A kit comprising:
(a) the VENTANA SP142 anti-PD-L1 diagnostic antibody; and
(b) the Dako 22C3 anti-PD-L1 diagnostic antibody, the VENTANA SP263 anti-PD-L1 diagnostic antibody, or the 28-8 anti-PD-L1 diagnostic antibody.

89. The kit of embodiment 88, further comprising one or more reagents for visualizing the anti-PD-L1 diagnostic antibodies of (a) and (b).

90. A PD-1 axis binding antagonist for use in treating a cancer in a patient, wherein the PD-1 axis binding antagonist is to be administered to the patient according to a treatment regimen comprising a PD-1 axis binding antagonist, wherein a tumor sample obtained from the patient has been determined to have:
(i) a first score obtained from applying a first scoring algorithm to a first stained portion of the tumor sample that meets or exceeds a first cutoff, wherein the first stained portion of the tumor sample was stained with an immune-directed PD-L1 assay; and
(ii) a second score obtained from applying a second scoring algorithm to a second stained portion of the tumor sample that meets or exceeds a second cutoff, wherein the second stained portion of the tumor sample was stained with an immune-agnostic PD-L1 assay.

91. Use of a PD-1 axis binding antagonist in the manufacture of a medicament for treating a cancer in a patient, wherein the PD-1 axis binding antagonist is to be administered to the patient according to a treatment regimen comprising a PD-1 axis binding antagonist, wherein a tumor sample obtained from the patient has been determined to have:
(i) a first score obtained from applying a first scoring algorithm to a first stained portion of the tumor sample that meets or exceeds a first cutoff, wherein the first stained portion of the tumor sample was stained with an immune-directed PD-L1 assay; and
(ii) a second score obtained from applying a second scoring algorithm to a second stained portion of the tumor sample that meets or exceeds a second cutoff, wherein the second stained portion of the tumor sample was stained with an immune-agnostic PD-L1 assay.

92. Use of a PD-1 axis binding antagonist for treating a cancer in a patient, wherein the PD-1 axis binding antagonist is to be administered to the patient according to a treatment regimen comprising a PD-1 axis binding antagonist, wherein a tumor sample obtained from the patient has been determined to have:
(i) a first score obtained from applying a first scoring algorithm to a first stained portion of the tumor sample that meets or exceeds a first cutoff, wherein the first stained portion of the tumor sample was stained with an immune-directed PD-L1 assay; and
(ii) a second score obtained from applying a second scoring algorithm to a second stained portion of the tumor sample that meets or exceeds a second cutoff, wherein the second stained portion of the tumor sample was stained with an immune-agnostic PD-L1 assay.

93. The PD-1 axis binding antagonist for use or use of any one of embodiments 90-92, wherein the immune-directed PD-L1 assay has:
(i) at least an 80%, at least an 85%, at least a 90%, or at least a 95% overall percent agreement (OPA) with an SP142 Assay using the first scoring algorithm at the first cutoff value;
(ii) at least an 80%, at least an 85%, at least a 90%, or at least a 95% positive percent agreement (PPA) with an SP142 Assay using the first scoring algorithm at the first cutoff value;
(iii) at least an 80%, at least an 85%, at least a 90%, or at least a 95% negative percent agreement (NPA) with an SP142 Assay using the first scoring algorithm at the first cutoff value;
(iv) at least an 80%, at least an 85%, at least a 90%, or at least a 95% PPA and at least an 80%, at least an 85%, at least a 90%, or at least a 95% NPA with an SP142 Assay using the first scoring algorithm at the first cutoff value;
(v) at least an 80%, at least an 85%, at least a 90%, or at least a 95% PPA and at least an 80%, at least an 85%, at least a 90%, or at least a 95% OPA with an SP142 Assay using the first scoring algorithm at the first cutoff value;
(vi) at least an 80%, at least an 85%, at least a 90%, or at least a 95% NPA and at least an 80%, at least an 85%, at least a 90%, or at least a 95% OPA with an SP142 Assay using the first scoring algorithm at the first cutoff value; and/or
(vii) at least an 80%, at least an 85%, at least a 90%, or at least a 95% OPA, at least an 80%, at least an 85%, at least a 90%, or at least a 95% PPA, and at least an 80%, at least an 85%, at least a 90%, or at least a 95% NPA with an SP142 Assay using the first scoring algorithm at the first cutoff value.

94. The PD-1 axis binding antagonist for use or use of any one of embodiments 90-93, wherein the immune-agnostic PD-L1 assay has:
(i) at least an 80%, at least an 85%, at least a 90%, or at least a 95% OPA with an 22C3 Assay using the second scoring algorithm at the second cutoff value;
(ii) at least an 80%, at least an 85%, at least a 90%, or at least a 95% PPA with an 22C3 Assay using the second scoring algorithm at the second cutoff value;
(iii) at least an 80%, at least an 85%, at least a 90%, or at least a 95% NPA with an 22C3 Assay using the second scoring algorithm at the second cutoff value;
(iv) at least an 80%, at least an 85%, at least a 90%, or at least a 95% PPA and at least an 80%, at least an 85%, at least a 90%, or at least a 95% NPA with an 22C3 Assay using the second scoring algorithm at the second cutoff value;
(v) at least an 80%, at least an 85%, at least a 90%, or at least a 95% PPA and at least an 80%, at least an 85%, at least a 90%, or at least a 95% OPA with an 22C3 Assay using the second scoring algorithm at the second cutoff value;
(vi) at least an 80%, at least an 85%, at least a 90%, or at least a 95% NPA and at least an 80%, at least an 85%, at least a 90%, or at least a 95% OPA with an 22C3 Assay using the second scoring algorithm at the second cutoff value; and/or
(vii) at least an 80%, at least an 85%, at least a 90%, or at least a 95% OPA, at least an 80%, at least an 85%, at least a 90%, or at least a 95% PPA, and at least an 80%, at least an 85%, at least a 90%, or at least a 95% NPA with an 22C3 Assay using the second scoring algorithm at the second cutoff value.

95. The PD-1 axis binding antagonist for use or use of any one of embodiments 90-94, wherein the immune-directed PD-L1 assay comprises a PD-L1 immunohistochemical (IHC) assay comprising the VENTANA SP142 anti-PD-L1 diagnostic antibody.

96. The PD-1 axis binding antagonist for use or use of any one of embodiments 90-95, wherein the immune-agnostic PD-L1 assay comprises a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody.

97. The PD-1 axis binding antagonist for use or use of any one of embodiments 90-96, wherein the first cutoff is a detectable expression level of PD-L1 in tumor-infiltrating immune cells that comprise 5% or more of the tumor sample.

98. The PD-1 axis binding antagonist for use or use of any one of embodiments 90-97, wherein the second cutoff is a CPS ≥10%.

99. The PD-1 axis binding antagonist for use or use of any one of embodiments 90-98, wherein the tumor sample is an FFPE tumor sample, an archival tumor sample, a fresh tumor sample, or a frozen tumor sample.

100. The PD-1 axis binding antagonist for use or use of any one of embodiments 90-99, wherein the cancer is a bladder cancer, a kidney cancer, a lung cancer, a cancer of the urinary tract, a breast cancer, a prostate cancer, a cancer of the peritoneum, a hepatocellular cancer, a gastric or stomach cancer, a pancreatic cancer, a glioblastoma, a cervical cancer, an ovarian cancer, a liver cancer, a hepatoma, a colon cancer, a rectal cancer, a colorectal cancer, an endometrial or uterine carcinoma, a salivary gland carcinoma, a prostate cancer, a vulval cancer, a thyroid cancer, a hepatic carcinoma, an anal carcinoma, a penile carcinoma, a melanoma, a multiple myeloma or B-cell lymphoma, a chronic lymphocytic leukemia (CLL), an acute lymphoblastic leukemia (ALL), an acute myologenous leukemia (AML), a hairy cell leukemia, a chronic myeloblastic leukemia (CML), a post-transplant lymphoproliferative disorder (PTLD), a myelodysplastic syndrome (MDS), Meigs' syndrome, a brain cancer, or a head and neck cancer.

101. The PD-1 axis binding antagonist for use or use of embodiment 100, wherein the cancer is a bladder cancer.

102. The PD-1 axis binding antagonist for use or use of embodiment 101, wherein the bladder cancer is a urothelial carcinoma (UC).

103. The PD-1 axis binding antagonist for use or use of embodiment 102, wherein the UC is a locally advanced or metastatic UC.

104. The PD-1 axis binding antagonist for use or use of embodiment 103, wherein the patient is previously untreated for the locally advanced or metastatic UC.

105. The PD-1 axis binding antagonist for use or use of embodiment 103 or 104, wherein the locally advanced or metastatic UC is histologically documented, locally advanced (T4b, any N; or any T, N2-3) or metastatic urothelial carcinoma (mUC) (M1, Stage IV).

106. The PD-1 axis binding antagonist for use or use of any one of embodiments 103-105, wherein the cancer is locally advanced UC.

107. The PD-1 axis binding antagonist for use or use of any one of embodiments 103-106, wherein the locally advanced UC is inoperable.

108. The PD-1 axis binding antagonist for use or use of any one of embodiments 103-105, wherein the cancer is metastatic UC.

109. The PD-1 axis binding antagonist for use or use of any one of embodiments 103-108, wherein the patient is eligible for treatment with a platinum-based chemotherapy.

110. The PD-1 axis binding antagonist for use or use of embodiment 109, wherein the patient is eligible for treatment with a cisplatin-based chemotherapy.

111. The PD-1 axis binding antagonist for use or use of embodiments 103-110, wherein the patient has not received prior chemotherapy for the locally advanced or metastatic UC.

112. The PD-1 axis binding antagonist for use or use of any one of embodiments 103-110, wherein the patient has previously received an adjuvant or neoadjuvant chemotherapy or chemoradiation for UC, and has had a treatment-free interval of more than 12 months between the last administration of the adjuvant or neoadjuvant chemotherapy or chemoradiation and the date of recurrence.

113. The PD-1 axis binding antagonist for use or use of any one of embodiments 90-112, wherein the PD-1 axis binding antagonist is a PD-L1 binding antagonist, a PD-1 binding antagonist, or a PD-L2 binding antagonist.

114. The PD-1 axis binding antagonist for use or use of embodiment 113, wherein the PD-1 axis binding antagonist is a PD-L1 binding antagonist.

115. The PD-1 axis binding antagonist for use or use of embodiment 114, wherein the PD-1 axis binding antagonist is an anti-PD-L1 antibody.

116. The PD-1 axis binding antagonist for use or use of embodiment 115, wherein the anti-PD-L1 antibody comprises the following HVRs:
(i) an HVR-H1 sequence of GFTFSDSWIH (SEQ ID NO: 3);
(ii) an HVR-H2 sequence of AWISPYGGSTYYADSVKG (SEQ ID NO: 4);
(iii) an HVR-H3 sequence of RHWPGGFDY (SEQ ID NO: 5);
(iv) an HVR-L1 sequence of RASQDVSTAVA (SEQ ID NO: 6);
(v) an HVR-L2 sequence of SASFLYS (SEQ ID NO: 7); and
(vi) an HVR-L3 sequence of QQYLYHPAT (SEQ ID NO: 8).

117. The PD-1 axis binding antagonist for use or use of embodiment 115 or 116, wherein the anti-PD-L1 antibody comprises:
(a) a VH domain comprising the amino acid sequence of SEQ ID NO: 9; and
(b) a VL domain comprising the amino acid sequence of SEQ ID NO: 10.

118. The PD-1 axis binding antagonist for use or use of any one of embodiments 115-117, wherein the anti-PD-L1 antibody is atezolizumab.

119. The PD-1 axis binding antagonist for use or use of embodiment 118, wherein atezolizumab is to be administered to the patient intravenously at a dose of about 840 mg every 2 weeks, about 1200 mg every 3 weeks, or about 1680 mg every 4 weeks.

120. The PD-1 axis binding antagonist for use or use of embodiment 119, wherein atezolizumab is to be administered to the patient intravenously at a dose of about 1200 mg every 3 weeks.

121. The PD-1 axis binding antagonist for use or use of embodiment 120, wherein atezolizumab is to be administered to the patient in 21-day dosing cycles, and wherein atezolizumab is to be administered to the patient intravenously at a dose of about 1200 mg on Day-2 to Day 4 of each 21-day dosing cycle.

122. The PD-1 axis binding antagonist for use or use of embodiment 121, wherein atezolizumab is to be administered to the patient intravenously at a dose of about 1200 mg on Day 1 of each 21-day dosing cycle.

123. The PD-1 axis binding antagonist for use or use of any one of embodiments 90-122, wherein the PD-1 axis binding antagonist is to be administered to the patient as a monotherapy.

124. The PD-1 axis binding antagonist for use or use of any one of embodiments 90-122, wherein the PD-1 axis binding antagonist is to be administered to the patient in combination with one or more additional therapeutic agents.

125. The PD-1 axis binding antagonist for use or use of embodiment 124, wherein the one or more additional therapeutic agents comprise a platinum-based chemotherapy.

126. The PD-1 axis binding antagonist for use or use of embodiment 125, wherein the platinum-based chemotherapy comprises a platinum-based chemotherapeutic agent and a nucleoside analog.

127. The PD-1 axis binding antagonist for use or use of embodiment 126, wherein the platinum-based chemotherapeutic agent is cisplatin, carboplatin, or oxaliplatin.

128. The PD-1 axis binding antagonist for use or use of embodiment 127, wherein the platinum-based chemotherapeutic agent is cisplatin.

129. The PD-1 axis binding antagonist for use or use of embodiment 127, wherein the platinum-based chemotherapeutic agent is carboplatin.

130. The PD-1 axis binding antagonist for use or use of any one of embodiments 126-129, wherein the nucleoside analog is gemcitabine.

131. The PD-1 axis binding antagonist for use or use of any one of embodiments 126-130, wherein the platinum-based chemotherapy comprises cisplatin and gemcitabine or carboplatin and gemcitabine.

132. The PD-1 axis binding antagonist for use or use of embodiment 131, wherein the platinum-based chemotherapy comprises cisplatin and gemcitabine.

133. The PD-1 axis binding antagonist for use or use of embodiment 131, wherein the platinum-based chemotherapy comprises carboplatin and gemcitabine.

134. The PD-1 axis binding antagonist for use or use of any one of embodiments 90-133, wherein the patient has been identified as one who may benefit from the treatment regimen comprising a PD-1 axis binding antagonist, and wherein the benefit from the treatment regimen comprising the PD-1 axis binding antagonist is in terms of overall survival (OS).

135. The PD-1 axis binding antagonist for use or use of embodiment 134, wherein the treatment regimen extends the patient's OS by from about 5.7 months to about 17 months as compared to treatment with a platinum-based chemotherapy without the PD-1 axis binding antagonist.

136. The PD-1 axis binding antagonist for use or use of embodiment 135, wherein the treatment regimen extends the patient's OS by about 11.3 months as compared to treatment with a platinum-based chemotherapy without the PD-1 axis binding antagonist.

137. The PD-1 axis binding antagonist for use or use of any one of embodiments 90-136, wherein the patient is a human.

138. The PD-1 axis binding antagonist for use or use of any one of embodiments 90-137, wherein the tumor sample obtained from the patient has the presence of discernible PD-L1 staining of any intensity in tumor-infiltrating immune cells covering >5% of tumor area occupied by tumor cells, associated intratumoral, and contiguous peritumoral stroma, as determined by the PD-L1 IHC assay comprising the VENTANA SP142 anti-PD-L1 diagnostic antibody.

139. The PD-1 axis binding antagonist for use or use of any one of embodiments 90-137, wherein the tumor sample obtained from the patient has a CPS of ≥10 using a PD-L1 IHC assay comprising the Dako 22C3 anti-PD-L1 diagnostic antibody.

140. The PD-1 axis binding antagonist for use or use of any one of embodiments 90-137, wherein the tumor sample obtained from the patient has a CPS of ≥10 using a PD-L1 IHC assay comprising the VENTANA SP263 anti-PD-L1 diagnostic antibody.

141. The PD-1 axis binding antagonist for use or use of any one of embodiments 90-137, wherein the tumor sample obtained from the patient has a CPS of ≥10 using a PD-L1 IHC assay comprising the 28-8 anti-PD-L1 diagnostic antibody.

142. A method of treating a locally advanced or metastatic urothelial carcinoma (UC) in a patient in need thereof, wherein the patient is previously untreated for the locally advanced or metastatic UC, the method comprising administering to the patient a treatment regimen comprising an anti-PD-L1 antibody comprising the following hypervariable regions (HVRs):

(i) an HVR-H1 sequence of
(SEQ ID NO: 3)
GFTFSDSWIH;
(ii) an HVR-H2 sequence of
(SEQ ID NO: 4)
AWISPYGGSTYYADSVKG;
(iii) an HVR-H3 sequence of
(SEQ ID NO: 5)
RHWPGGFDY;
(iv) an HVR-L1 sequence of
(SEQ ID NO: 6)
RASQDVSTAVA;
(v) an HVR-L2 sequence of
(SEQ ID NO: 7)
SASFLYS;
and
(vi) an HVR-L3 sequence of
(SEQ ID NO: 8)
QQYLYHPAT,

wherein a tumor sample obtained from the patient has been determined to have:
a detectable expression level of PD-L1 in tumor-infiltrating immune cells that comprise 5% or more of the tumor sample using a PD-L1 immunohistochemical (IHC) assay comprising the VENTANA SP142 anti-PD-L1 diagnostic antibody; and
a Combined Positive Score (CPS) of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from the treatment regimen comprising the anti-PD-L1 antibody.

143. A method of treating a locally advanced or metastatic UC in a patient in need thereof, wherein the patient is previously untreated for the locally advanced or metastatic UC, the method comprising:
(a) determining that a tumor sample obtained from the patient has:
   a detectable expression level of PD-L1 in tumor-infiltrating immune cells that comprise 5% or more of the tumor sample using a PD-L1 IHC assay comprising the VENTANA SP142 anti-PD-L1 diagnostic antibody; and
   a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising an anti-PD-L1 antibody comprising the following HVRs:

(i) an HVR-H1 sequence of
(SEQ ID NO: 3)
GFTFSDSWIH;
(ii) an HVR-H2 sequence of
(SEQ ID NO: 4)
AWISPYGGSTYYADSVKG;
(iii) an HVR-H3 sequence of
(SEQ ID NO: 5)
RHWPGGFDY;
(iv) an HVR-L1 sequence of
(SEQ ID NO: 6)
RASQDVSTAVA;
(v) an HVR-L2 sequence of
(SEQ ID NO: 7)
SASFLYS;
and
(vi) an HVR-L3 sequence of
(SEQ ID NO: 8)
QQYLYHPAT;

and
(b) administering the treatment regimen comprising the anti-PD-L1 antibody to the patient identified in step (a) as one who may benefit from the treatment regimen comprising the anti-PD-L1 antibody.

144. A method of selecting a therapy for treating a locally advanced or metastatic UC in a patient in need thereof, wherein the patient is previously untreated for the locally advanced or metastatic UC, the method comprising:
(a) determining that a tumor sample obtained from the patient has:
   a detectable expression level of PD-L1 in tumor-infiltrating immune cells that comprise 5% or more of the tumor sample using a PD-L1 IHC assay comprising the VENTANA SP142 anti-PD-L1 diagnostic antibody; and
   a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising an anti-PD-L1 antibody comprising the following HVRs:

(i) an HVR-H1 sequence of
(SEQ ID NO: 3)
GFTFSDSWIH;
(ii) an HVR-H2 sequence of
(SEQ ID NO: 4)
AWISPYGGSTYYADSVKG;
(iii) an HVR-H3 sequence of
(SEQ ID NO: 5)
RHWPGGFDY;
(iv) an HVR-L1 sequence of
(SEQ ID NO: 6)
RASQDVSTAVA;
(v) an HVR-L2 sequence of
(SEQ ID NO: 7)
SASFLYS;
and
(vi) an HVR-L3 sequence of
(SEQ ID NO: 8)
QQYLYHPAT

and
(b) selecting a treatment regimen comprising the anti-PD-L1 antibody for the patient identified in step (a) as one who may benefit from the treatment regimen comprising the anti-PD-L1 antibody.

145. A method of identifying a patient having a locally advanced or metastatic UC who may benefit from a treatment regimen comprising an anti-PD-L1 antibody, wherein the patient is previously untreated for the locally advanced or metastatic UC, the method comprising:
determining that a tumor sample obtained from the patient has:
a detectable expression level of PD-L1 in tumor-infiltrating immune cells that comprise 5% or more of the tumor sample using a PD-L1 IHC assay comprising the VENTANA SP142 anti-PD-L1 diagnostic antibody; and
a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising an anti-PD-L1 antibody, wherein the anti-PD-L1 antibody comprises the following HVRs:

(i) an HVR-H1 sequence of
(SEQ ID NO: 3)
GFTFSDSWIH;
(ii) an HVR-H2 sequence of
(SEQ ID NO: 4)
AWISPYGGSTYYADSVKG;
(iii) an HVR-H3 sequence of
(SEQ ID NO: 5)
RHWPGGFDY;
(iv) an HVR-L1 sequence of
(SEQ ID NO: 6)
RASQDVSTAVA;
(v) an HVR-L2 sequence of
(SEQ ID NO: 7)
SASFLYS;
and
(vi) an HVR-L3 sequence of
(SEQ ID NO: 8)
QQYLYHPAT;

146. The method of embodiment 144 or 145, further comprising administering the treatment regimen comprising the anti-PD-L1 antibody to the patient.

147. The method of any one of embodiments 1-146, wherein the benefit from the treatment regimen comprising the anti-PD-L1 antibody is in terms of overall survival (OS).

148. The method of embodiment 147, wherein the treatment regimen extends the patient's OS by from about 5.7 months to about 17 months as compared to treatment with a platinum-based chemotherapy without the anti-PD-L1 antibody.

149. The method of embodiment 148, wherein the treatment regimen extends the patient's OS by about 11.3 months as compared to treatment with a platinum-based chemotherapy without the anti-PD-L1 antibody.

150. The method of embodiment 148 or 149, wherein the platinum-based chemotherapy comprises a platinum-based chemotherapeutic agent and a nucleoside analog.

151. The method of embodiment 150, wherein the platinum-based chemotherapeutic agent is cisplatin, carboplatin, or oxaliplatin.

152. The method of embodiment 151, wherein the platinum-based chemotherapeutic agent is cisplatin.

153. The method of embodiment 151, wherein the platinum-based chemotherapeutic agent is carboplatin.

154. The method of any one of embodiments 150-153, wherein the nucleoside analog is gemcitabine.

155. The method of any one of embodiments 149-154, wherein the platinum-based chemotherapy comprises cisplatin and gemcitabine or carboplatin and gemcitabine.

156. The method of embodiment 155, wherein the platinum-based chemotherapy comprises cisplatin and gemcitabine.

157. The method of embodiment 155, wherein the platinum-based chemotherapy comprises carboplatin and gemcitabine.

158. The method of any one of embodiments 142-157, wherein the anti-PD-L1 antibody comprises: (a) a VH domain comprising the amino acid sequence of SEQ ID NO: 9; and
(b) a VL domain comprising the amino acid sequence of SEQ ID NO: 10.

159. The method of any one of embodiments 142-158, wherein the anti-PD-L1 antibody is atezolizumab.

160. The method of embodiment 159, wherein atezolizumab is administered to the patient intravenously at a dose of about 840 mg every 2 weeks, about 1200 mg every 3 weeks, or about 1680 mg every 4 weeks.

161. The method of embodiment 160, wherein atezolizumab is administered to the patient intravenously at a dose of about 1200 mg every 3 weeks.

162. The method of embodiment 161, wherein atezolizumab is administered to the patient in 21-day dosing cycles, and wherein atezolizumab is administered to the patient intravenously at a dose of about 1200 mg on Day-2 to Day 4 of each 21-day dosing cycle.

163. The method of embodiment 162, wherein atezolizumab is administered to the patient intravenously at a dose of about 1200 mg on Day 1 of each 21-day dosing cycle.

164. The method of any one of embodiments 142-163, wherein the anti-PD-L1 antibody is administered to the patient as a monotherapy.

165. The method of any one of embodiments 142-163, wherein the anti-PD-L1 antibody is administered to the patient in combination with one or more additional therapeutic agents.

166. The method of embodiment 165, wherein the one or more additional therapeutic agents comprise a platinum-based chemotherapy.

167. The method of embodiment 166, wherein the platinum-based chemotherapy comprises a platinum-based chemotherapeutic agent and a nucleoside analog.

168. The method of embodiment 167, wherein the platinum-based chemotherapeutic agent is cisplatin, carboplatin, or oxaliplatin.

169. The method of embodiment 168, wherein the platinum-based chemotherapeutic agent is cisplatin.

170. The method of embodiment 168, wherein the platinum-based chemotherapeutic agent is carboplatin.

171. The method of any one of embodiments 167-170, wherein the nucleoside analog is gemcitabine.

172. The method of any one of embodiments 166-171, wherein the platinum-based chemotherapy comprises cisplatin and gemcitabine or carboplatin and gemcitabine.

173. The method of embodiment 172, wherein the platinum-based chemotherapy comprises cisplatin and gemcitabine.

174. The method of embodiment 172, wherein the platinum-based chemotherapy comprises carboplatin and gemcitabine.

175. The method of any one of embodiments 142-174, wherein the patient has not received prior chemotherapy for the locally advanced or metastatic UC.

176. The method of any one of embodiments 142-174, wherein the patient has previously received an adjuvant or neoadjuvant chemotherapy or chemoradiation for urothelial carcinoma, and has had a treatment-free interval of more than 12 months between the last administration of the adjuvant or neoadjuvant chemotherapy or chemoradiation and the date of recurrence.

177. The method of any one of embodiments 142-176, wherein the locally advanced or metastatic UC is histologically documented, locally advanced (T4b, any N; or any T, N2-3) or metastatic urothelial carcinoma (mUC) (M1, Stage IV).

178. The method of any one of embodiments 142-177, wherein the UC is locally advanced UC.

179. The method of any one of embodiments 142-178, wherein the locally advanced UC is inoperable.

180. The method of any one of embodiments 142-177, wherein the UC is metastatic UC.

181. The method of any one of embodiments 142-180, wherein the patient is eligible for treatment with a platinum-based chemotherapy.

182. The method of embodiment 181, wherein the patient is eligible for treatment with a cisplatin-based chemotherapy.

183. The method of any one of embodiments 142-182, wherein the patient is a human.

184. The method of any one of embodiments 142-183, wherein the tumor sample obtained from the patient has the presence of discernible PD-L1 staining of any intensity in tumor-infiltrating immune cells covering ≥5% of tumor area occupied by tumor cells, associated intratumoral, and contiguous peritumoral stroma, as determined by the PD-L1 IHC assay comprising the VENTANA SP142 anti-PD-L1 diagnostic antibody.

185. The method of any one of embodiments 142-184, wherein the tumor sample obtained from the patient has a CPS of ≥10 using a PD-L1 IHC assay comprising the Dako 22C3 anti-PD-L1 diagnostic antibody.

186. The method of any one of embodiments 142-184, wherein the tumor sample obtained from the patient has a CPS of ≥10 using a PD-L1 IHC assay comprising the VENTANA SP263 anti-PD-L1 diagnostic antibody.

187. The method of any one of embodiments 142-184, wherein the tumor sample obtained from the patient has a CPS of ≥10 using a PD-L1 IHC assay comprising the 28-8 anti-PD-L1 diagnostic antibody.

188. An anti-PD-L1 antibody for use in treatment of a locally advanced or metastatic UC in a patient in need thereof, wherein the patient is previously untreated for the locally advanced or metastatic UC, the treatment comprising administration to the patient of a treatment regimen comprising an anti-PD-L1 antibody comprising the following HVRs:

(i) an HVR-H1 sequence of
(SEQ ID NO: 3)
GFTFSDSWIH;
(ii) an HVR-H2 sequence of
(SEQ ID NO: 4)
AWISPYGGSTYYADSVKG;
(iii) an HVR-H3 sequence of
(SEQ ID NO: 5)
RHWPGGFDY;
(iv) an HVR-L1 sequence of
(SEQ ID NO: 6)
RASQDVSTAVA;
(v) an HVR-L2 sequence of
(SEQ ID NO: 7)
SASFLYS;
and
(vi) an HVR-L3 sequence of
(SEQ ID NO: 8)
QQYLYHPAT,

wherein a tumor sample obtained from the patient has been determined to have:
   a detectable expression level of PD-L1 in tumor-infiltrating immune cells that comprise 5% or more of the tumor sample using a PD-L1 IHC assay comprising the VENTANA SP142 anti-PD-L1 diagnostic antibody; and
   a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from the treatment regimen comprising the anti-PD-L1 antibody.

189. Use of an anti-PD-L1 antibody for the treatment of a locally advanced or metastatic UC in a patient in need thereof, wherein the patient is previously untreated for the locally advanced or metastatic UC, the treatment comprising administration to the patient of a treatment regimen comprising an anti-PD-L1 antibody comprising the following HVRs:

(i) an HVR-H1 sequence of
(SEQ ID NO: 3)
GFTFSDSWIH;
(ii) an HVR-H2 sequence of
(SEQ ID NO: 4)
AWISPYGGSTYYADSVKG;
(iii) an HVR-H3 sequence of
(SEQ ID NO: 5)
RHWPGGFDY;
(iv) an HVR-L1 sequence of
(SEQ ID NO: 6)
RASQDVSTAVA;
(v) an HVR-L2 sequence of
(SEQ ID NO: 7)
SASFLYS;
and
(vi) an HVR-L3 sequence of
(SEQ ID NO: 8)
QQYLYHPAT,

wherein a tumor sample obtained from the patient has been determined to have:
   a detectable expression level of PD-L1 in tumor-infiltrating immune cells that comprise 5% or more of the tumor sample using a PD-L1 IHC assay comprising the VENTANA SP142 anti-PD-L1 diagnostic antibody; and
   a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from the treatment regimen comprising the anti-PD-L1 antibody.

190. Use of an anti-PD-L1 antibody in the manufacture of a medicament for use in treatment of a locally advanced or metastatic UC in a patient in need thereof, wherein the patient is previously untreated for the locally advanced or metastatic UC, the treatment comprising administration to the patient of a treatment regimen comprising an anti-PD-L1 antibody comprising the following HVRs:

(i) an HVR-H1 sequence of
(SEQ ID NO: 3)
GFTFSDSWIH;
(ii) an HVR-H2 sequence of
(SEQ ID NO: 4)
AWISPYGGSTYYADSVKG;
(iii) an HVR-H3 sequence of
(SEQ ID NO: 5)
RHWPGGFDY;
(iv) an HVR-L1 sequence of
(SEQ ID NO: 6)
RASQDVSTAVA;
(v) an HVR-L2 sequence of
(SEQ ID NO: 7)
SASFLYS;
and
(vi) an HVR-L3 sequence of
(SEQ ID NO: 8)
QQYLYHPAT,

wherein a tumor sample obtained from the patient has been determined to have:
   a detectable expression level of PD-L1 in tumor-infiltrating immune cells that comprise 5% or more of the tumor sample using a PD-L1 IHC assay comprising the VENTANA SP142 anti-PD-L1 diagnostic antibody; and
   a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from the treatment regimen comprising the anti-PD-L1 antibody.

191. An anti-PD-L1 antibody for use in a method of treating a locally advanced or metastatic UC in a patient in need thereof, wherein the patient is previously untreated for the locally advanced or metastatic UC, the method comprising:
(a) determining that a tumor sample obtained from the patient has:
   a detectable expression level of PD-L1 in tumor-infiltrating immune cells that comprise 5% or more of the tumor sample using a PD-L1 IHC assay comprising the VENTANA SP142 anti-PD-L1 diagnostic antibody; and
   a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising an anti-PD-L1 antibody comprising the following HVRs:

(i) an HVR-H1 sequence of
(SEQ ID NO: 3)
GFTFSDSWIH;
(ii) an HVR-H2 sequence of
(SEQ ID NO: 4)
AWISPYGGSTYYADSVKG;
(iii) an HVR-H3 sequence of
(SEQ ID NO: 5)
RHWPGGFDY;
(iv) an HVR-L1 sequence of
(SEQ ID NO: 6)
RASQDVSTAVA;
(v) an HVR-L2 sequence of
(SEQ ID NO: 7)
SASFLYS;
and
(vi) an HVR-L3 sequence of
(SEQ ID NO: 8)
QQYLYHPAT;

and
(b) administering the treatment regimen comprising the anti-PD-L1 antibody to the patient identified in step (a) as one who may benefit from the treatment regimen comprising the anti-PD-L1 antibody.

192. Use of an anti-PD-L1 antibody in a method of treating a locally advanced or metastatic UC in a patient in need thereof, wherein the patient is previously untreated for the locally advanced or metastatic UC, the method comprising:
(b) determining that a tumor sample obtained from the patient has:
   a detectable expression level of PD-L1 in tumor-infiltrating immune cells that comprise 5% or more of the tumor sample using a PD-L1 IHC assay comprising the VENTANA SP142 anti-PD-L1 diagnostic antibody; and
   a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising an anti-PD-L1 antibody comprising the following HVRs:

(i) an HVR-H1 sequence of
(SEQ ID NO: 3)
GFTFSDSWIH;
(ii) an HVR-H2 sequence of
(SEQ ID NO: 4)
AWISPYGGSTYYADSVKG;
(iii) an HVR-H3 sequence of
(SEQ ID NO: 5)
RHWPGGFDY;
(iv) an HVR-L1 sequence of
(SEQ ID NO: 6)
RASQDVSTAVA;
(v) an HVR-L2 sequence of
(SEQ ID NO: 7)
SASFLYS;
and
(vi) an HVR-L3 sequence of
(SEQ ID NO: 8)
QQYLYHPAT;

and
(b) administering the treatment regimen comprising the anti-PD-L1 antibody to the patient identified in step (a) as one who may benefit from the treatment regimen comprising the anti-PD-L1 antibody.

193. Use of an anti-PD-L1 antibody in the manufacture of a medicament for use in a method of treating a locally advanced or metastatic UC in a patient in need thereof, wherein the patient is previously untreated for the locally advanced or metastatic UC, the method comprising:
(c) determining that a tumor sample obtained from the patient has:
   a detectable expression level of PD-L1 in tumor-infiltrating immune cells that comprise 5% or more of the tumor sample using a PD-L1 IHC assay comprising the VENTANA SP142 anti-PD-L1 diagnostic antibody; and
   a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody, thereby identifying the patient as one who may benefit from a treatment regimen comprising an anti-PD-L1 antibody comprising the following HVRs:

(i) an HVR-H1 sequence of
(SEQ ID NO: 3)
GFTFSDSWIH;
(ii) an HVR-H2 sequence of
(SEQ ID NO: 4)
AWISPYGGSTYYADSVKG;
(iii) an HVR-H3 sequence of
(SEQ ID NO: 5)
RHWPGGFDY;
(iv) an HVR-L1 sequence of
(SEQ ID NO: 6)
RASQDVSTAVA;
(v) an HVR-L2 sequence of
(SEQ ID NO: 7)
SASFLYS;
and
(vi) an HVR-L3 sequence of
(SEQ ID NO: 8)
QQYLYHPAT;

and
(b) administering the treatment regimen comprising the anti-PD-L1 antibody to the patient identified in step (a) as one who may benefit from the treatment regimen comprising the anti-PD-L1 antibody.

194. The anti-PD-L1 antibody for use or use of any one of embodiments 188-193, wherein the benefit from the treatment regimen comprising the anti-PD-L1 antibody is in terms of OS.

195. The anti-PD-L1 antibody for use or use of embodiment 194, wherein the treatment regimen extends the patient's OS by from about 5.7 months to about 17 months as compared to treatment with a platinum-based chemotherapy without the anti-PD-L1 antibody.

196. The anti-PD-L1 antibody for use or use of embodiment 195, wherein the treatment regimen extends the patient's OS by about 11.3 months as compared to treatment with a platinum-based chemotherapy without the anti-PD-L1 antibody.

197. The anti-PD-L1 antibody for use or use of embodiment 195 or 196, wherein the platinum-based chemotherapy comprises a platinum-based chemotherapeutic agent and a nucleoside analog.

198. The anti-PD-L1 antibody for use or use of embodiment 197, wherein the platinum-based chemotherapeutic agent is cisplatin, carboplatin, or oxaliplatin.

199. The anti-PD-L1 antibody for use or use of embodiment 198, wherein the platinum-based chemotherapeutic agent is cisplatin.

200. The anti-PD-L1 antibody for use or use of embodiment 198, wherein the platinum-based chemotherapeutic agent is carboplatin.

201. The anti-PD-L1 antibody for use or use of any one of embodiments 197-200, wherein the nucleoside analog is gemcitabine.

202. The anti-PD-L1 antibody for use or use of any one of embodiments 196-201, wherein the platinum-based chemotherapy comprises cisplatin and gemcitabine or carboplatin and gemcitabine.

203. The anti-PD-L1 antibody for use or use of embodiment 202, wherein the platinum-based chemotherapy comprises cisplatin and gemcitabine.

204. The anti-PD-L1 antibody for use or use of embodiment 202, wherein the platinum-based chemotherapy comprises carboplatin and gemcitabine.

205. The anti-PD-L1 antibody for use or use of any one of embodiments 188-204, wherein the anti-PD-L1 antibody comprises:
(a) a VH domain comprising the amino acid sequence of SEQ ID NO: 9; and
(b) a VL domain comprising the amino acid sequence of SEQ ID NO: 10.

206. The anti-PD-L1 antibody for use or use of any one of embodiments 188-205, wherein the anti-PD-L1 antibody is atezolizumab.

207. The anti-PD-L1 antibody for use or use of embodiment 206, wherein atezolizumab is administered to the patient intravenously at a dose of about 840 mg every 2 weeks, about 1200 mg every 3 weeks, or about 1680 mg every 4 weeks.

208. The anti-PD-L1 antibody for use or use of embodiment 207, wherein atezolizumab is administered to the patient intravenously at a dose of about 1200 mg every 3 weeks.

209. The anti-PD-L1 antibody for use or use of embodiment 208, wherein atezolizumab is administered to the patient in 21-day dosing cycles, and wherein atezolizumab is administered to the patient intravenously at a dose of about 1200 mg on Day-2 to Day 4 of each 21-day dosing cycle.

210. The anti-PD-L1 antibody for use or use of embodiment 209, wherein atezolizumab is administered to the patient intravenously at a dose of about 1200 mg on Day 1 of each 21-day dosing cycle.

211. The anti-PD-L1 antibody for use or use of any one of embodiments 188-210, wherein the anti-PD-L1 antibody is administered to the patient as a monotherapy.

212. The anti-PD-L1 antibody for use or use of any one of embodiments 188-210, wherein the anti-PD-L1 antibody is administered to the patient in combination with one or more additional therapeutic agents.

213. The anti-PD-L1 antibody for use or use of embodiment 212, wherein the one or more additional therapeutic agents comprise a platinum-based chemotherapy.

214. The anti-PD-L1 antibody for use or use of embodiment 213, wherein the platinum-based chemotherapy comprises a platinum-based chemotherapeutic agent and a nucleoside analog.

215. The anti-PD-L1 antibody for use or use of embodiment 214, wherein the platinum-based chemotherapeutic agent is cisplatin, carboplatin, or oxaliplatin.

216. The anti-PD-L1 antibody for use or use of embodiment 215, wherein the platinum-based chemotherapeutic agent is cisplatin.

217. The anti-PD-L1 antibody for use or use of embodiment 215, wherein the platinum-based chemotherapeutic agent is carboplatin.

218. The anti-PD-L1 antibody for use or use of any one of embodiments 214-217, wherein the nucleoside analog is gemcitabine.

219. The anti-PD-L1 antibody for use or use of any one of embodiments 213-218, wherein the platinum-based chemotherapy comprises cisplatin and gemcitabine or carboplatin and gemcitabine. 220. The anti-PD-L1 antibody for use or use of embodiment 219, wherein the platinum-based chemotherapy comprises cisplatin and gemcitabine.

221. The anti-PD-L1 antibody for use or use of embodiment 219, wherein the platinum-based chemotherapy comprises carboplatin and gemcitabine.

222. The anti-PD-L1 antibody for use or use of any one of embodiments 188-221, wherein the patient has not received prior chemotherapy for the locally advanced or metastatic UC.

223. The anti-PD-L1 antibody for use or use of any one of embodiments 188-222, wherein the patient has previously received an adjuvant or neoadjuvant chemotherapy or chemoradiation for urothelial carcinoma, and has had a treatment-free interval of more than 12 months between the last administration of the adjuvant or neoadjuvant chemotherapy or chemoradiation and the date of recurrence.

224. The anti-PD-L1 antibody for use or use of any one of embodiments 188-223, wherein the locally advanced or metastatic UC is histologically documented, locally advanced (T4b, any N; or any T, N2-3) or mUC (M1, Stage IV).

225. The anti-PD-L1 antibody for use or use of any one of embodiments 188-224, wherein the UC is locally advanced UC.

226. The anti-PD-L1 antibody for use or use of any one of embodiments 188-225, wherein the locally advanced UC is inoperable.

227. The anti-PD-L1 antibody for use or use of any one of embodiments 188-224, wherein the UC is metastatic UC.

228. The anti-PD-L1 antibody for use or use of any one of embodiments 188-227, wherein the patient is eligible for treatment with a platinum-based chemotherapy.

229. The anti-PD-L1 antibody for use or use of embodiment 228, wherein the patient is eligible for treatment with a cisplatin-based chemotherapy.

230. The anti-PD-L1 antibody for use or use of any one of embodiments 188-229, wherein the patient is a human.

231. The anti-PD-L1 antibody for use or use of any one of embodiments 188-230, wherein the tumor sample obtained from the patient has the presence of discernible PD-L1 staining of any intensity in tumor-infiltrating immune cells covering ≥5% of tumor area occupied by tumor cells, associated intratumoral, and contiguous peritumoral stroma, as determined by the PD-L1 IHC assay comprising the VENTANA SP142 anti-PD-L1 diagnostic antibody.

232. The anti-PD-L1 antibody for use or use of any one of embodiments 188-231, wherein the tumor sample obtained from the patient has a CPS of ≥10 using a PD-L1 IHC assay comprising the Dako 22C3 anti-PD-L1 diagnostic antibody.

233. The anti-PD-L1 antibody for use or use of any one of embodiments 188-231, wherein the tumor sample obtained from the patient has a CPS of ≥10 using a PD-L1 IHC assay comprising the VENTANA SP263 anti-PD-L1 diagnostic antibody.

234. The anti-PD-L1 antibody for use or use of any one of embodiments 188-231, wherein the tumor sample obtained from the patient has a CPS of ≥10 using a PD-L1 IHC assay comprising the 28-8 anti-PD-L1 diagnostic antibody.

235. The anti-PD-L1 antibody for use or use of any one of embodiments 188-234, wherein the tumor sample is a formalin-fixed and paraffin-embedded (FFPE) tumor sample, an archival tumor sample, a fresh tumor sample, or a frozen tumor sample.

236. An assay for patient determining the presence or expression level of PD-L1 in a tumor sample obtained from a patient suffering from a cancer, the assay comprising:
(a) determining the presence or expression level of PD-L1 in a tumor sample obtained from the patient using a PD-L1 IHC assay comprising the VENTANA SP142 anti-PD-L1 diagnostic antibody; and
(b) determining the presence or expression level of PD-L1 in the tumor sample obtained from the patient using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody.

237. The assay of embodiment 236, wherein the tumor sample obtained from the patient has a detectable expression level of PD-L1 in tumor-infiltrating immune cells that comprise 5% or more of the tumor sample using a PD-L1 IHC assay comprising the VENTANA SP142 anti-PD-L1 diagnostic antibody.

238. The assay of embodiment 236 or 237, wherein the tumor sample obtained from the patient has the presence of discernible PD-L1 staining of any intensity in tumor-infiltrating immune cells covering ≥5% of tumor area occupied by tumor cells, associated intratumoral, and contiguous peritumoral stroma, as determined by the PD-L1 IHC assay comprising the VENTANA SP142 anti-PD-L1 diagnostic antibody.

239. The assay of any one of embodiments 236-238, wherein the tumor sample obtained from the patient has a CPS of ≥10 using a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody.

240. The assay of any one of embodiments 236-239, wherein the tumor sample obtained from the patient has:
a detectable expression level of PD-L1 in tumor-infiltrating immune cells that comprise 5% or more of the tumor sample using the PD-L1 IHC assay comprising the VENTANA SP142 anti-PD-L1 diagnostic antibody; and
a CPS of ≥10 using the PD-L1 IHC assay comprising the Dako 22C3 anti-PD-L1 diagnostic antibody.

241. The assay of any one of embodiment 236-240, wherein steps (a) and (b) are performed simultaneously.

242. The assay of any one of embodiment 236-241, wherein steps (a) and (b) are performed sequentially.

243. The assay of any one of embodiments 236-242, wherein steps (a) and (b) are performed in different sections of the tumor sample or in the same section of the tumor sample. 244. The assay of embodiment 243, wherein the different sections of the tumor sample are consecutive sections.

245. The assay of any one of embodiments 236-244, wherein the cancer is locally advanced or metastatic urothelial carcinoma.

246. The assay of embodiment 245, wherein the patient is previously untreated for the locally advanced or metastatic urothelial carcinoma.

247. The assay of any one of embodiments 236-246, wherein the assay is used for (i) selecting a therapy for treating a locally advanced or metastatic UC in a patient in need thereof or (ii) identifying a patient having a locally advanced or metastatic UC who may benefit from a treatment regimen comprising an anti-PD-L1 antibody.

248. The assay of embodiment 247, wherein the anti-PD-L1 antibody comprises the following HVRs:

(i) an HVR-H1 sequence of
(SEQ ID NO: 3)
GFTFSDSWIH;
(ii) an HVR-H2 sequence of
(SEQ ID NO: 4)
AWISPYGGSTYYADSVKG;
(iii) an HVR-H3 sequence of
(SEQ ID NO: 5)
RHWPGGFDY;
(iv) an HVR-L1 sequence of
(SEQ ID NO: 6)
RASQDVSTAVA;
(v) an HVR-L2 sequence of
(SEQ ID NO: 7)
SASFLYS;
and
(vi) an HVR-L3 sequence of
(SEQ ID NO: 8)
QQYLYHPAT.

249. The assay of any one of embodiments 246-248, wherein the tumor sample is an FFPE tumor sample, an archival tumor sample, a fresh tumor sample, or a frozen tumor sample.

250. A method of labeling PD-L1 in a tumor sample, the method comprising the following steps:
(a) contacting the tumor sample with the VENTANA SP142 anti-PD-L1 diagnostic antibody;
(b) contacting the tumor sample with the Dako 22C3 anti-PD-L1 diagnostic antibody, the VENTANA SP263 anti-PD-L1 diagnostic antibody, or the 28-8 anti-PD-L1 diagnostic antibody; and
(c) visualizing the anti-PD-L1 diagnostic antibodies of steps (a) and (b) with one or more detectable reagents that generates a detectable signal for both of the anti-PD-L1 diagnostic antibodies.

251. The method of embodiment 250, wherein the detectable signal for the VENTANA SP142 anti-PD-L1 diagnostic antibody is an amplified signal.

252. The method of embodiment 251, wherein the amplified signal is generated by tyramide signal amplification.

253. The method of any one of embodiment 250-252, wherein steps (a) and (b) are performed simultaneously.

254. The method of any one of embodiment 250-252, wherein steps (a) and (b) are performed sequentially.

255. The method of any one of embodiments 250-254, wherein steps (a) and (b) are performed in different sections of the tumor sample or in the same section of the tumor sample.

256. The method of embodiment 255, wherein the different sections of the tumor sample are consecutive sections.

257. The method of any one of embodiments 250-256, wherein the visualizing comprises IHC or immunofluorescence (IF).

258. The method of embodiment 257, wherein the visualizing comprises IHC.

259. The method of any one of embodiments 142-187 or 250-258, wherein the tumor sample is an FFPE tumor sample, an archival tumor sample, a fresh tumor sample, or a frozen tumor sample.

260. The method of any one of embodiments 250-259, wherein the tumor sample is obtained from a patient having a cancer.

261. The method of embodiment 260, wherein the cancer is locally advanced or metastatic urothelial carcinoma.

262. The method of embodiment 261, wherein the patient is previously untreated for the locally advanced or metastatic urothelial carcinoma.

263. A kit comprising:
(a) the VENTANA SP142 anti-PD-L1 diagnostic antibody; and
(b) the Dako 22C3 anti-PD-L1 diagnostic antibody, the VENTANA SP263 anti-PD-L1 diagnostic antibody, or the 28-8 anti-PD-L1 diagnostic antibody.

264. The kit of embodiment 263, further comprising one or more reagents for visualizing the anti-PD-L1 diagnostic antibodies of (a) and (b).

265. A method of identifying a tumor likely to respond to a PD-1 axis binding antagonist, the method comprising:
(a) staining a first portion of the tumor with an immune-directed PD-L1 assay to obtain a first stained sample;
(b) generating a first score by applying a first scoring algorithm to the first stained sample;
(c) staining a second portion of the tumor with an immune-agnostic PD-L1 assay to obtain a second stained sample;
(d) generating a second score by applying a second scoring algorithm the second stained sample; and
(e) comparing the first score to a first cutoff and the second score to a second cutoff, wherein the tumor is likely to respond to the PD-1 axis binding antagonist when both the first score meets or exceeds the first cutoff and the second score meets or exceeds the second cutoff.

266. The method of embodiment 265, wherein the immune-directed PD-L1 assay has:
(i) at least an 80% overall percent agreement (OPA) with an SP142 Assay using the first scoring algorithm at the first cutoff value;
(ii) at least an 80% positive percent agreement (PPA) with an SP142 Assay using the first scoring algorithm at the first cutoff value;
(iii) at least an 80% negative percent agreement (NPA) with an SP142 Assay using the first scoring algorithm at the first cutoff value;
(iv) at least an 80% PPA and at least an 80% NPA with an SP142 Assay using the first scoring algorithm at the first cutoff value;
(v) at least an 80% PPA and at least an 80% OPA with an SP142 Assay using the first scoring algorithm at the first cutoff value;
(vi) at least an 80% NPA and at least an 80% OPA with an SP142 Assay using the first scoring algorithm at the first cutoff value; and/or
(vii) at least an 80% OPA, at least an 80% PPA, and at least an 80% NPA with an SP142 Assay using the first scoring algorithm at the first cutoff value.

267. The method of embodiment 265 or 266, wherein the immune-agnostic PD-L1 assay has:
(i) at least an 80% overall percent agreement (OPA) with an 22C3 Assay using the second scoring algorithm at the second cutoff value;
(ii) at least an 80% positive percent agreement (PPA) with an 22C3 Assay using the second scoring algorithm at the second cutoff value;
(iii) at least an 80% negative percent agreement (NPA) with an 22C3 Assay using the second scoring algorithm at the second cutoff value;
(iv) at least an 80% PPA and at least an 80% NPA with an 22C3 Assay using the second scoring algorithm at the second cutoff value;
(v) at least an 80% PPA and at least an 80% OPA with an 22C3 Assay using the second scoring algorithm at the second cutoff value;
(vi) at least an 80% NPA and at least an 80% OPA with an 22C3 Assay using the second scoring algorithm at the second cutoff value; and/or
(vii) at least an 80% OPA, at least an 80% PPA, and at least an 80% NPA with an 22C3 Assay using the second scoring algorithm at the second cutoff value.

268. A method of stratifying a tumor having a score with an immune-agnostic PD-L1 assay that exceeds a pre-determined cutoff, the method comprising:
(a) staining a portion of the tumor with an immune-directed PD-L1 assay to obtain a stained sample;
(b) generating a score by applying a scoring algorithm to the stained sample; and
(c) comparing the score to a first cutoff, wherein the tumor is likely to respond to a PD-1 axis binding antagonist when both the first score meets or exceeds the first cutoff.

269. A method of stratifying a CPS ≥10% tumor as determined by a 22C3 assay, the method comprising:
(a) staining a portion of the tumor with an immune-directed PD-L1 assay to obtain a stained sample;
(b) generating a score by applying a scoring algorithm to the stained sample; and
(c) comparing the score to a first cutoff and the second score to a second cutoff, wherein the tumor is likely to respond to the PD-1 axis binding antagonist when both the first score meets or exceeds the first cutoff and the second score meets or exceeds the second cutoff.

270. A method of stratifying a CPS ≥10% tumor as determined by an SP263 assay, the method comprising:
(a) staining a portion of the tumor with an immune-directed PD-L1 assay to obtain a stained sample;
(b) generating a score by applying a scoring algorithm to the stained sample; and
(c) comparing the score to a first cutoff and the second score to a second cutoff, wherein the tumor is likely to respond to the PD-1 axis binding antagonist when both the first score meets or exceeds the first cutoff and the second score meets or exceeds the second cutoff.

271. A method of stratifying a CPS ≥10% tumor as determined by a 28-8 assay, the method comprising:
(a) staining a portion of the tumor with an immune-directed PD-L1 assay to obtain a stained sample;
(b) generating a score by applying a scoring algorithm to the stained sample; and
(c) comparing the score to a first cutoff and the second score to a second cutoff, wherein the tumor is likely to respond to the PD-1 axis binding antagonist when both the first score meets or exceeds the first cutoff and the second score meets or exceeds the second cutoff.

272. The method of any one of embodiments 265-281, wherein the tumor is a locally advanced or metastatic UC.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: A Phase III, Multicenter, Randomized, Placebo-Controlled Study of Atezolizumab (Anti-PD-L1 Antibody) as Monotherapy and in Combination with Platinum-based Chemotherapy in Patients with Untreated Locally Advanced or Metastatic Urothelial Carcinoma

Inoperable locally advanced or metastatic urothelial carcinoma (UC) is generally a uniformly lethal disease with high unmet medical need. With little to no advancement in therapeutic landscape for patients over the last 30 years, the survival for these patients regardless of time of diagnosis or treatment received remains stagnant with no major improvement over the last 20 years. Platinum-based chemotherapy is the current standard-of-care for patients with previously untreated metastatic urothelial carcinoma. For medically fit patients with metastatic urothelial carcinoma, cisplatin-based combination chemotherapy is the preferred initial therapy. Despite the observed efficacy of cisplatin-based combination chemotherapy, up to 50% of patients are medically “unfit” or ineligible to receive cisplatin because of baseline comorbidities. A consensus working group has defined cisplatin-ineligibility under specific criteria (Galsky et al. Lancet. Oncol. 12:211-4, 2011, which is incorporated by reference herein in its entirety), including performance status, renal function, hearing-loss history, peripheral neuropathy, and cardiac function. For patients who are ineligible to receive cisplatin, treatment options include carboplatin-based or non-platinum-based combinations, single-agent regimens, and best supportive care (BSC).

Atezolizumab (also known as MPDL3280A) is a humanized IgG1 monoclonal antibody consisting of two heavy chains (448 amino acids) and two light chains (214 amino acids) and is produced in Chinese hamster ovary cells. Atezolizumab was engineered to eliminate Fc-effector function via a single amino acid substitution (asparagine to alanine) at position 298 on the heavy chain, which results in a non-glycosylated antibody that has minimal binding to Fc receptors and prevents Fc-effector function at expected concentrations in humans. Atezolizumab targets human programmed death-ligand 1 (PD-L1) and inhibits its interaction with its receptors, programmed death-1 (PD-1) and B7.1 (CD80, B7-1). Both of these interactions are reported to provide inhibitory signals to T cells.

This Example describes the IMvigor130 study, which is a Phase III, multicenter, randomized, placebo-controlled, partially blind study designed to evaluate the efficacy of atezolizumab given as monotherapy or in combination with platinum-based chemotherapy versus placebo in combination with platinum-based chemotherapy in patients with locally advanced or metastatic urothelial carcinoma who have not received prior systemic therapy.

### A. Objectives and Endpoints

i. Primary Efficacy Objective

The primary efficacy objective for this study is to evaluate the efficacy of atezolizumab plus platinum-based chemotherapy compared with placebo plus platinum-based chemotherapy on the basis of the following endpoints:
Co-primary endpoints of progression-free survival (PFS) and overall survival (OS)
   PFS is defined as the time from randomization to the first documented disease progression as determined by the investigator with use of RECIST v1.1, or death due to any cause, whichever occurs first.
   OS is defined as the time from randomization to death due to any cause.

In addition, a primary efficacy objective is to evaluate the efficacy of atezolizumab monotherapy compared with placebo plus platinum-based chemotherapy on the basis of OS, as defined above.

ii. Secondary Efficacy Objectives

The secondary efficacy objectives for this study are to evaluate the efficacy of atezolizumab given as either monotherapy or in combination with platinum-based chemotherapy compared with placebo in combination with platinum-based chemotherapy on the basis of the following endpoints:
ORR, defined as the proportion of patients with a confirmed objective response, either complete response (CR) or partial response (PR), observed on two assessments ≥28 days apart per RECIST v1.1, based on investigator assessment.
Duration of response (DOR), defined for patients with an objective response as the time from the first documented objective response to documented disease progression per RECIST v1.1, based on investigator assessment, or death due to any cause, whichever occurs first.
IRF-PFS, defined as the time from randomization to the first documented disease progression as determined by blinded independent central review with use of RECIST v1.1, or death due to any cause, whichever occurs first.
Investigator-assessed (INV-PFS) in patients treated with atezolizumab monotherapy compared with patients treated with placebo plus platinum-based chemotherapy
OS rate at 1 year
PFS rate at 1 year.
Time to deterioration in global health status as measured by the European Organization for Research and Treatment of Cancer EORTC Quality-of-Life Questionnaire Core 30 (QLQ-C30).
Time to deterioration in physical function as measured by the EORTC QLQ-C30

### Exploratory Efficacy Objectives

The exploratory efficacy objectives for this study are to evaluate the efficacy of atezolizumab given as either monotherapy or in combination with platinum-based chemotherapy compared with placebo in combination with platinum-based chemotherapy on the basis of the following endpoints:
Disease control rate (DCR), defined as the proportion of patients with confirmed CR or PR as best response, or stable disease maintained for ≥6 months, per RECIST v1.1.
Relationship between tumor tissue PD-L1 expression and measures of efficacy
Predictive, prognostic, and pharmacodynamic exploratory biomarkers in archival and/or fresh tumor tissue and blood and their association with disease status and/or response to study treatment.
Disease and treatment burden as measured by the symptom (e.g., pain, fatigue) and function scores from the QLQ C30

An additional exploratory objective is to characterize patients who are able to continue treatment past progression by RECIST v1.1 as permitted per protocol and to describe clinical outcomes by treatment arm using modified RECIST, such as ORR, DOR, DCR, and PFS.

iv. Safety Objectives

The safety objectives for this study are to evaluate the safety and tolerability of atezolizumab given as either monotherapy or in combination with platinum-based chemotherapy compared with placebo plus platinum-based chemotherapy on the basis of the following:
Incidence, nature, and severity of adverse events graded according to National Cancer Institute (NCI) Common Terminology Criteria for Adverse Events (CTCAE) v4.0
Changes in vital signs, and clinical laboratory results

### V. Pharmacokinetic Objective

The PK objective for this study is to characterize the pharmacokinetics of atezolizumab when administered as monotherapy or in combination with platinum-based chemotherapy in patients who are treatment-naive:
PK parameters for atezolizumab include maximum serum concentration (Cₘₐₓ) and minimum serum concentration (Cₘᵢₙ) when appropriate, as data allow.

### B. Study Design

IMvigor130 (clinicaltrials.gov identifier: NCT02807636) is a Phase III, multicenter, randomized, placebo-controlled, partially blind study designed to evaluate the safety and efficacy of atezolizumab given as monotherapy or in combination with platinum-based chemotherapy versus placebo in combination with platinum-based chemotherapy in patients with locally advanced or metastatic urothelial carcinoma who have not received prior systemic therapy (see FIG. 1).

Treatment with platinum-based chemotherapy continued until progressive disease (PD) per RECIST v1.1 or unacceptable toxicity per investigator discretion. In the case of CR, only two more cycles of platinum-based chemotherapy were administered after the response confirmation. In specific circumstances, treatment was allowed to continue beyond disease progression.

No crossover was allowed from the control arm to either experimental arm (Arms A and B).

Patients were eligible to receive treatment with platinum-based chemotherapy (either gemcitabine with cisplatin or gemcitabine with carboplatin) and had measurable disease, defined by RECIST v1.1. Note, the study was initially implemented with randomization to two treatment arms (carboplatin plus gemcitabine with or without atezolizumab) in patients who were ineligible for cisplatin-based chemotherapy. A third treatment arm was added (open-label atezolizumab monotherapy). In addition, the eligibility criteria were expanded to also allow patients who are eligible for cisplatin-based chemotherapy to enter the study.

Tumor specimens from eligible patients were prospectively tested for PD-L1 IHC expression by a central laboratory prior to randomization. The IHC scores have three categories (IC0, IC1, IC2/3). The study enrolled all eligible patients whose tissue is evaluable for expression testing, regardless of PD-L1 expression status.

Patients were randomized in a 1:1:1 ratio with a permuted block method and an interactive voice-web response system to receive one of the following:
Arm A (experimental arm): blinded atezolizumab in combination with open-label platinum-based chemotherapy (gemcitabine with either cisplatin or carboplatin)
Arm B (experimental arm): open-label atezolizumab monotherapy
Arm C (control arm): blinded placebo in combination with open-label platinum-based chemotherapy (gemcitabine with either cisplatin or carboplatin).

Prior to randomization, the investigator selected which chemotherapy the patient would receive (gemcitabine and carboplatin vs. gemcitabine and cisplatin) if the patient was randomized to Arm A or Arm C. The Galsky criteria (see Galsky et al. 2011, supra) were used to guide determination of cisplatin ineligibility.

Randomization was stratified by the following factors:
PD-L1 status (IC0 (<1%) vs. IC1 (≥1% and <5%) vs. IC2/3 (≥5%)).
Bajorin model risk factor score/liver metastasis (0 vs. 1 vs. 2 or patients with liver metastasis).
Investigator-determined chemotherapy (gemcitabine and carboplatin vs. gemcitabine and cisplatin).

Gemcitabine was administered at a dose of 1000 mg/m² by intravenous (IV) infusion on Day 1 and Day 8 of each 21-day cycle.

Carboplatin was administered at area under the concentration-time curve (AUC) 4.5 by IV infusion on Day 1 of each 21-day cycle.

Cisplatin was administered at a dose of 70 mg/m² by IV infusion on Day 1 of each 21-day cycle. Split dosing of cisplatin was not permitted.

All visits and infusions were allowed to be administered with a window of +3 days, except for the Day 8 visit. The Day 8 dose of gemcitabine was allowed to be given no earlier than Day 7 but was allowed to be given up to Day 11 (−1 to +3) of a cycle.

Gemcitabine and platinum chemotherapy (carboplatin or cisplatin) was administered until investigator-assessed disease progression per RECIST v1.1 or unacceptable toxicity.

For patients who received chemotherapy and who had a CR, only two more cycles of chemotherapy were administered after the response confirmation. For patients who received chemotherapy and who had a PR or stable disease, chemotherapy administration was allowed to be discontinued after completion of six cycles, if necessary, to comply with institutional guidelines.

If the initial protocol doses of chemotherapy agents differed from institutional guidelines or local label, the initial doses were allowed to be modified to achieve compliance.

Atezolizumab was administered at a fixed dose of 1200 mg by IV infusion on Day 1 of each 21-day cycle until investigator-assessed disease progression per RECIST v1.1. Placebo for atezolizumab (Arm C) will be administered by IV infusion on Day 1 of each 21-day cycle until investigator-assessed disease progression per RECIST v1.1. In specific circumstances, treatment was allowed continue beyond disease progression. No crossover was allowed from the control arm to either experimental arm (Arms A and B).

Patients underwent scheduled tumor assessment at baseline and every 9 weeks (from initiation of the first dose) thereafter for 54 weeks after randomization. After 54 weeks, patients underwent tumor assessment every 12 weeks until disease progression per RECIST v1.1, death, study termination, or withdrawal of consent, whichever occurred first. Patients discontinued treatment at the first occurrence of radiographic progression, per RECIST v1.1, with the following exception: Patients who have achieved a PR or CR of target lesions and who develop new lesions (≤3) that were amenable to surgery or ablative techniques (e.g., radiotherapy, radiofrequency ablation) were allowed to continue to receive study treatment if they demonstrated no further progression of disease at the next imaging assessment and continued to demonstrate clinical benefit per investigator. Because gemcitabine is not indicated for use in combination with radiation therapy and has been shown to cause excess toxicity (mucositis, pneumonitis) as well as radiation recall when administered in close proximity to radiation therapy, patients who continued treatment with gemcitabine were recommended to not receive radiation to ports that include a significant proportion of lung or mucosal surface (e.g., esophagus or intestine). In the absence of disease progression, tumor assessments were expected to continue, regardless of whether patients started new anti-cancer therapy, until death, loss to follow-up, withdrawal of consent, or study termination. Follow-up data capture, including subsequent anti-cancer therapies (including targeted therapies and immunotherapies), will continue for each patient until death, loss to follow-up, withdrawal of consent, or study termination, whichever occurred first.

Patients were required to undergo tumor biopsy sample collection, if clinically feasible as assessed by investigators, at the first evidence of radiographic disease progression. These data were used to confirm that radiographic findings are consistent with the presence of tumor. In addition, these data were analyzed for the association between changes in tumor tissue and clinical outcome and to understand further the potential mechanisms of resistance to treatment.

Safety assessments included the incidence, nature, and severity of adverse events, vital signs and laboratory abnormalities graded per the NCI CTCAE v4.0. Laboratory safety assessments included the regular monitoring of hematology and blood chemistry. Serum samples were collected to monitor atezolizumab pharmacokinetics and to detect the presence of antibodies to atezolizumab. Patient samples, including archival tumor tissues, as well as serum, plasma, and blood, were collected for future exploratory biomarker assessments. Adverse events of special interest (AESIs) that required immediate reporting included the following conditions, which may be suggestive of an autoimmune disorder: cases of potential drug-induced liver injury that include an elevated alanine aminotransferase (ALT) or aspartate aminotransferase (AST) in combination with either elevated bilirubin or clinical jaundice as defined by Hy's law, suspected transmission of an infectious agent by the study drug (defined as any organism, virus, or infectious particle (e.g., prion protein-transmitting transmissible spongiform encephalopathy), pathogenic or nonpathogenic (transmission of an infectious agent may be suspected from clinical symptoms or laboratory findings that indicate an infection in a patient exposed to a medicinal product; this term applies only when a contamination of the study drug is suspected)), pneumonitis, colitis, endocrinopathies (diabetes mellitus, pancreatitis, adrenal insufficiency, hyperthyroidism, and hypophysitis), hepatitis (including AST or ALT >10× upper limit of normal), systemic lupus erythematosus, neurological disorders (Guillain-Barre syndrome, myasthenic syndrome or myasthenia gravis, meningoencephalitis), events suggestive of hypersensitivity, infusion-related reactions, cytokine release, influenza-like illness, systemic inflammatory response syndrome, systemic immune activation, nephritis, ocular toxicities (e.g., uveitis, retinitis), myositis, myopathies (including rhabdomyolysis), and grade ≥2 cardiac disorders (e.g., atrial fibrillation, myocarditis, pericarditis).

This study was initially implemented with randomization to 2 treatment arms (carboplatin plus gemcitabine with or without atezolizumab) in patients who were ineligible for cisplatin-based chemotherapy. A protocol amendment (version 3 (v3)) added an open-label atezolizumab monotherapy arm and expanded the eligibility criteria to also allow enrollment of patients who were eligible for cisplatin-based chemotherapy. This protocol amendment was implemented while recruitment was ongoing and, as a result of this design change, the study consists of 2 stages: patients recruited into the study prior to v3 (Stage 1) and patients recruited after v3 (Stage 2) (see FIG. 2). Patients from both stages were included in the final analysis.

Later during recruitment, based on an ad hoc review of the survival data, the study independent data monitoring committee (IDMC) recommended closure of the atezolizumab monotherapy arm to further accrual of all patients with a PD-L1 expression status of IC0 or IC1 (at this point, enrollment into Stage 1 was complete). The IDMC did not recommend a change in therapy for patients already randomized to atezolizumab monotherapy on study. The IDMC recommendation also stated that patients with a PD-L1 expression status of IC2/3 could continue to be randomized to each of the 3 arms and that Arms A and C should remain unchanged (open to all patients regardless of PD-L1 status). This recommendation was implemented in protocol amendment v6. Stratified randomization to the 3 arms in a 1:1:1 ratio continued regardless of PD-L1 status. For all new patients randomized to Arm B after approval of v6, PD-L1 status was unblinded to the investigator and patient at the time of randomization, and patients whose PD-L1 expression status was IC2/3 received atezolizumab monotherapy; however, patients with a PD-L1 expression status of IC0 or IC1 received chemotherapy combined with atezolizumab instead of atezolizumab monotherapy. No change was recommended for patients currently enrolled in the atezolizumab monotherapy arm at the time of protocol amendment v6. If patients or providers wished to change treatment, patients were requested to still continue on survival follow-up.

### C. Materials and Methods

i. Patients

Patients with locally advanced or metastatic urothelial carcinoma who have not received prior systemic therapy for urothelial carcinoma were enrolled in the global enrollment phase of the study.

ii. Inclusion Criteria

Patients were required to meet the following criteria for study entry:
Considered to be eligible to receive platinum-based chemotherapy, in the investigator's judgment
ECOG performance status of ≤2.
Histologically documented, locally advanced (T4b, any N; or any T, N 2-3) or metastatic urothelial carcinoma (M1, Stage IV) (also termed TCC or UCC of the urinary tract; including renal pelvis, ureters, urinary bladder, and urethra)
   Patients with mixed histologies were required to have a dominant transitional cell pattern.
   Locally advanced bladder cancer was required to be inoperable on the basis of involvement of pelvic sidewall or adjacent viscera (clinical Stage T4b) or bulky nodal metastasis (N2-N3).
Representative formalin-fixed paraffin-embedded (FFPE) tumor specimens in paraffin blocks (blocks preferred) or at least 15 unstained slides, with an associated pathology report, for central testing and determined to be evaluable for tumor PD-L1 expression prior to study enrollment; patients who had fewer than 15 unstained slides available at baseline (but no fewer than 10) were potentially eligible following discussion with the Medical Monitor.
No prior chemotherapy for inoperable, locally advanced, or metastatic urothelial carcinoma.
   For patients who received prior adjuvant/neoadjuvant chemotherapy or chemo-radiation for urothelial carcinoma, a treatment-free interval >12 months between the last treatment administration and the date of recurrence was required in order to be considered treatment naive in the metastatic setting.
   Prior local intravesical chemotherapy or immunotherapy was allowed if completed at least 4 weeks prior to the initiation of study treatment.
Measurable disease, as defined by RECIST v1.1
   Previously irradiated lesions were not be counted as target lesions unless there was demonstrated progression in the lesion since radiotherapy and no other lesions are available for selection as target lesions.

Prior to randomization, the investigator determined which chemotherapy was appropriate for the patient (gemcitabine and carboplatin or gemcitabine and cisplatin). Ineligibility to receive cisplatin-based chemotherapy was based upon the criteria published by Galsky et al. supra. Patients who met at least one of the following criteria were recommended to be considered for treatment with carboplatin plus gemcitabine if randomized to Arm A or Arm C; however, the final decision was made by the investigator:
Impaired renal function (glomerular filtration rate (GFR)>30 but <60 mL/min); GFR should be assessed by direct measurement (i.e., creatinine clearance or ethyldediaminetetra-acetate) or, if not available, by calculation from serum/plasma creatinine (Cockcroft-Gault formula).
NCI CTCAE v4.0 Grade ≥2 audiometric hearing loss of 25 decibels at two contiguous frequencies
NCI CTCAE v4.0 Grade ≥2 peripheral neuropathy (i.e., sensory alteration or paresthesia, including tingling)
ECOG performance status of 2
   iii. Study Treatment

The investigational medicinal products (IMPs) for this study are atezolizumab, placebo, gemcitabine, carboplatin, and cisplatin. Patients were expected to receive their first dose of study treatment no later than 5 calendar days after randomization. All visits and infusions were allowed to be administered with a window of ±3 days, except for the Day 8 visit. The Day 8 dose of gemcitabine was allowed to be given no earlier than Day 7 but was allowed be given up to Day 11 (−1 to +3) of a cycle.

The dose level of atezolizumab in this study was 1200 mg administered by IV infusion q3w. Administration of atezolizumab and placebo was performed in a setting with emergency medical facilities and staff who are trained to monitor for and respond to medical emergencies.

Gemcitabine was administered according to the local prescribing information. The starting dose of gemcitabine was 1000 mg/m², administered by IV infusion on Day 1 and Day 8 of each 21-day cycle. Day 8 gemcitabine administration was expected to not occur earlier than Day 7, but was allowed to occur up to Day 11. A change of body weight of +5% compared to previous measured weight required that the dose be re-calculated. Anti-emetic prophylaxis was allowed to be administered at the treating physician's discretion according to local practice.

Carboplatin was administered according to the local prescribing information. The starting dose of carboplatin was calculated to achieve an AUC of 4.5, administered by IV infusion on Day 1 of each 21-day cycle. If institutional guidelines conflicted with study protocol dosing, carboplatin was allowed to be administered at a maximum starting AUC of 5.0. A change of body weight of +5% compared to previous measured weight required that the dose be re-calculated. Carboplatin-based chemotherapy is considered to be moderately emetogenic, and appropriate anti-emetic prophylaxis was recommended, and use of a nonsteroidal anti-emetic regimen consisting of 5-HT3 receptor and NK1R antagonists was encouraged if feasible.

Patients were allowed to switch from carboplatin to cisplatin chemotherapy in the event the patient becomes eligible to receive cisplatin-based therapy, i.e., the patient does not meet any of the cisplatin-ineligibility criteria described above. Change in protocol chemotherapy was not allowed at the time of disease progression by RECIST v1.1.

Cisplatin was administered according to the local prescribing information. The starting dose of cisplatin was 70 mg/m², administered by IV infusion on Day 1 of each 21-day cycle. Split dosing of cisplatin was not permitted. A change of body weight of ±5% compared to previous measured weight required that the dose be re-calculated. Potential side effects from cisplatin include cumulative nephrotoxicity, myelosuppression, nausea, and vomiting. The use of a nonsteroidal anti-emetic regimen consisting of 5-HT3 receptor and NK1R antagonists was encouraged if feasible.

Patients will be allowed to switch from cisplatin to carboplatin chemotherapy if they become ineligible for cisplatin due to toxicity, or from carboplatin to cisplatin chemotherapy in the event that patient becomes eligible to receive cisplatin. Changes in protocol chemotherapy will not be allowed for the reason of suspected or confirmed disease progression by RECIST v1.1.

Note: If the initial protocol doses of gemcitabine, carboplatin, and cisplatin differed from institutional guidelines or local label, the initial doses were allowed to be modified to achieve compliance.

Gemcitabine and platinum chemotherapy (carboplatin or cisplatin) will be administered until investigator-assessed disease progression per RECIST v1.1 or unacceptable toxicity.

For patients in the combination arms who receive chemotherapy (Arms A and C) and who have a CR, only two more cycles of chemotherapy will be administered after the response confirmation.

For patients in the combination arms who receive chemotherapy and who have a PR or stable disease, chemotherapy administration may be discontinued after completion of six cycles, if necessary, to comply with institutional guidelines.

iv. Statistical Analysis

This study had 2 stages (FIG. 3). Stage 1 was implemented with 2 treatment arms (Arms A and C) in cisplatin-ineligible patients. A protocol amendment added a third treatment arm (Arm B) and expanded eligibility to cisplatin-eligible patients (Stage 2, FIG. 3).

For the comparison of efficacy in Arm A vs. Arm C, the intention-to-treat population was defined as all patients randomized to Arm A or Arm C in Stages 1 and 2, whether or not the assigned study treatment was received. For the comparison of efficacy in Arm B vs. Arm C, the intention-to-treat population included only patients concurrently enrolled in Stage 2 as well as those randomized at the time of the protocol amendment that limited Arm B enrollment to patients with PD-L1 IC2/3 tumors.

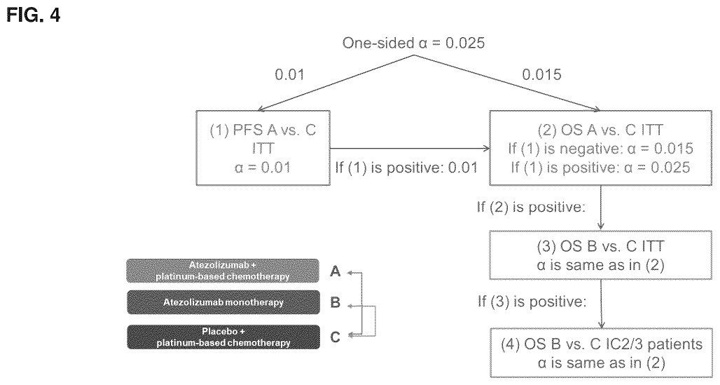

PFS and OS analyses were performed on the respective intention-to-treat populations for each treatment-arm comparison according to a pre-planned hierarchal hypothesis (FIG. 4). Formal treatment comparisons of OS in Arm A vs. Arm C and Arm B vs. Arm C were to be performed in a hierarchal fashion (FIG. 4). PFS and OS were compared between trial groups using the stratified log-rank test. For comparing Arm A with Arm C, the inverse normal combination test approach was used to take into account a possible effect of study stage. Kaplan-Meier methodology was used to estimate the survival functions for PFS and OS as well as the median time for each group from randomization to disease progression or death (for PFS) and median time from randomization to death (for OS). The safety population was defined as patients who received any amount of any component of study treatment. For purposes of safety evaluation, patients were analyzed according to the treatment received.

### Example 2: Assessment of PD-L1 Status

Expression of PD-L1 was assessed using the VENTANA SP142 IHC assay performed according to the manufacturer's instructions. The IC and TC IHC diagnostic criteria for the VENTANA SP142 IHC assay are described in Tables 2 and 3, respectively.

TABLE 2
Tumor-infiltrating immune cell (IC) IHC diagnostic criteria

| PD-L1 Diagnostic Assessment | IC Score |
|---|---|
| Absence of any discernible PD-L1 staining | IC0 |
| OR | |
| Presence of discernible PD-L1 staining of any | |
| intensity in tumor-infiltrating immune cells | |
| covering <1% of tumor area occupied by tumor cells, | |
| associated intratumoral stroma, and contiguous | |
| peri-tumoral desmoplastic stroma | |
| Presence of discernible PD-L1 staining of any | IC1 |
| intensity in tumor-infiltrating immune cells | |
| covering ≥1% to <5% of tumor area occupied by tumor | |
| cells, associated intratumoral stroma, and contiguous | |
| peri-tumoral desmoplastic stroma | |
| Presence of discernible PD-L1 staining of any | IC2 |
| intensity in tumor-infiltrating immune cells | |
| covering ≥5% to <10% of tumor area occupied by tumor | |
| cells, associated intratumoral stroma, and | |
| contiguous peri-tumoral desmoplastic stroma | |
| Presence of discernible PD-L1 staining of any | IC3 |
| intensity in tumor-infiltrating immune cells | |
| covering ≥10% of tumor area occupied by tumor cells, | |
| associated intratumoral stroma, and contiguous | |
| peri-tumoral desmoplastic stroma | |

TABLE 3
Tumor cell (TC) IHC diagnostic criteria

| PD-L1 Diagnostic Assessment | TC Score |
|---|---|
| Absence of any discernible PD-L1 staining | TC0 |
| OR | |
| Presence of discernible PD-L1 staining of any | |
| intensity in <1% of tumor cells | |
| Presence of discernible PD-L1 staining of any | TC1 |
| intensity in ≥1% to <5% of tumor cells | |
| Presence of discernible PD-L1 staining of any | TC2 |
| intensity in ≥5% to <50% of tumor cells | |
| Presence of discernible PD-L1 staining of any | TC3 |
| intensity in ≥50% of tumor cells | |

Detection of SP142 may include use of the Optiview DAB IHC Detection Kit and/or the Optiview Amplification Kit.

### OptiView DAB IHC Detection Kit

The OptiView DAB IHC Detection Kit is an indirect, biotin-free system for detecting mouse IgG, mouse IgM, and rabbit primary antibodies. The kit detects specific mouse and rabbit primary antibodies bound to an antigen in formalin-fixed, paraffin-embedded (FFPE) or frozen tissue sections and is intended to identify targets by immunohistochemistry (IHC) in sections of FFPE and frozen tissue that are stained on the VENTANA automated slide stainers and visualized by light microscopy. The specific antibody is located by a specific secondary antibody that is bound by an enzyme-labeled tertiary antibody. The complex is then visualized utilizing a precipitating enzyme product.

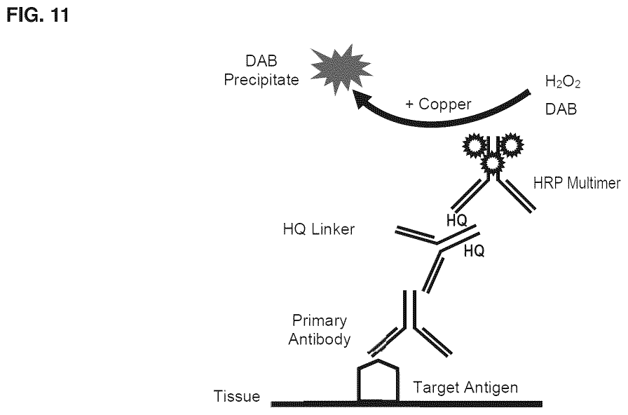

The OptiView DAB IHC Detection Kit utilizes a cocktail of secondary antibodies that locate the bound primary antibody. The cocktail secondary is recognized by an enzyme-bound tertiary antibody that is visualized with hydrogen peroxide substrate and 3,3′-diaminobenzidine tetrahydrochloride (DAB) chromogen, which produces a brown precipitate that is readily detected by light microscopy (see FIG. 11). The staining protocol consists of numerous steps in which reagents are incubated for pre-determined times at specific temperatures. At the end of each incubation step, the BenchMark IHC/ISH instrument washes the sections to remove unbound material and applies a liquid coverslip which minimizes the evaporation of the aqueous reagents from the slide. Results are interpreted using a light microscope.

### A. Materials

OptiView DAB IHC Detection Kit contains sufficient reagent for 250 tests. The following materials are each provided in the OptiView DAB IHC Detection Kit:
One 25 mL dispenser of OptiView Peroxidase Inhibitor contains 3.0% hydrogen peroxide solution.
One 25 mL dispenser of OptiView HQ Universal Linker contains a cocktail of HQ-labeled (HQ is a proprietary hapten covalently attached to the goat antibodies) antibodies (goat anti-mouse IgG, goat anti-mouse IgM, and goat anti-rabbit) (approximately 50 μg/mL) in a buffer containing protein with 0.05% ProClin 300, a preservative.
One 25 mL dispenser of OptiView HRP Multimer contains a mouse monoclonal anti-HQ-labeled HRP tertiary antibody (approximately 40 μg/mL) in a buffer containing protein with 0.05% ProClin 300, a preservative.
One 25 mL dispenser of OptiView DAB contains 0.2% w/v 3,3′-diaminobenzidine tetrahydrochloride (DAB) in a proprietary stabilizer solution with a proprietary preservative.
One 25 mL dispenser of OptiView H₂O₂ contains 0.04% hydrogen peroxide in a phosphate buffer solution.
One 25 mL dispenser of OptiView Copper contains copper sulfate (5.0 g/L) in an acetate buffer with a proprietary preservative.

### B. Specimen Collection and Preparation for Analysis

FFPE tissues are suitable for use with OptiView DAB IHC Detection Kit and BenchMark IHC/ISH instruments. The recommended tissue fixative is 10% neutral buffered formalin (NBF). Variable results may occur as a result of tissue section thickness, fixation type, incomplete or prolonged fixation or special processes such as decalcification of bone marrow preparations.

Each section should be cut to the appropriate thickness (2-5 μm) for the primary antibody being used and placed on a positively charged glass microscope slide. Slides containing the tissue section should dry in an upright position for at least 15 minutes at room temperature to drain excess water from beneath the section before baking. The slides may be baked/heated for one hour in a 60° C.±5° C. oven, or air dried at 37° C. for up to 24 hours. Slide drying and heating is used to dry the tissue post slide mounting and to enhance tissue adhesion to the glass. Extended heating of the tissue might result in decreased antigen availability.

Frozen sections should be cut to the appropriate thickness (4-5 μm) for the primary antibody being used, picked up on a glass microscope slide and immediately placed in cold acetone (4-8° C.) for ten minutes. Cytospins should be created using standard cytocentrifugation and fixation methods. The sections should be air dried for a minimum of 30 minutes and preferably overnight. Properly fixed and embedded tissues expressing the antigen will remain stable if stored in a cool location (15-25° C.).

### C. Procedure

OptiView DAB IHC Detection Kit has been developed for use on BenchMark IHC/ISH instruments in combination with VENTANA primary antibodies and ancillaries. The procedures for staining on BenchMark IHC/ISH instruments are as follows:
1. Apply slide bar code label which corresponds to the protocol to be performed.
2. Load the primary antibody, appropriate detection kit dispensers, and required accessory reagent onto the reagent tray and place them on the instrument.
3. Check bulk fluids and empty waste.
4. Load the slides onto the instrument.
5. Start the staining run.
6. At the completion of the run, remove the slides from the instrument.
7. Proceed to recommended post-instrument processing procedures, which are as follows:
   i. Wash slides in a mild dishwashing detergent to remove the coverslip solution.
   ii. Rinse slides thoroughly in distilled water to remove all detergent.
   iii. Dehydrate, clear, and coverslip with permanent mounting media.

### OptiView Amplification Kit

OptiView Amplification Kit may be used in conjunction with the OptiView DAB IHC Detection Kit to increase the staining intensity of mouse and rabbit primary antibodies. The kit is to be used for qualitative staining of formalin-fixed, paraffin-embedded tissue, or frozen tissue on a BenchMark IHC/ISH instrument with VENTANA primary antibodies and ancillary reagents, visualized by light microscopy.

The OptiView Amplification Kit may be used with the OptiView DAB IHC Detection Kit, rabbit and mouse primary antibodies, and BenchMark IHC/ISH instruments to achieve preferred immunohistochemistry (IHC) staining. The OptiView Amplification Kit includes an HQ hapten conjugate (OptiView Amplifier), corresponding substrate (OptiView Amplification H₂O₂), and mouse anti-HQ monoclonal antibody containing HRP (OptiView Amplification Multimer).

When the amplification option is selected in the OptiView DAB IHC Detection Kit protocol, following the deposition of the OptiView HRP Multimer reagent, the OptiView Amplifier and OptiView Amplification H₂O₂ are added to the slide. After an incubation step the slide is washed and OptiView Amplification Multimer is added and incubated. Following a wash step the OptiView DAB and OptiView H₂O₂ is added to the slide followed by the OptiView Copper reagent. The bound specific antibodies to antigens in the tissue are visualized by a brown colored precipitate.

### A. Materials

OptiView Amplification Kit contains sufficient reagent for 50 or 250 tests. The following materials are provided in the OptiView Amplification Kit which contains sufficient reagent for 250 tests:
One 25 mL dispenser of OptiView Amplifier contains tyramide with a linker containing HQ (<40 μg/mL) in a diluent with preservative.
One 25 mL dispenser of OptiView Amplification Multimer contains a mouse monoclonal anti-HQ-labeled HRP antibody (<10 μg/mL) in a diluent with 0.05% ProClin 300, a preservative.
One 25 mL dispenser of OptiView Amplification H₂O₂ contains 0.04% hydrogen peroxide.

### B. Procedure

The OptiView Amplification Kit is designed to provide an increase in signal generation for primary antibodies stained with the OptiView DAB IHC Detection Kit on BenchMark IHC/ISH instruments. To enable the use of the OptiView Amplification Kit, the user must load the reagents on the instrument, select the Amplification option in the OptiView procedure and establish incubation times for the Amplifier and Amplification Multimer steps. The amplification steps are inserted into the OptiView DAB IHC Detection Kit staining protocol.

### Example 3: PD-L1 Expression on Immune Cells by SP142 Co-Localizes with Dendritic Cells and is Associated with Improved Overall Survival with Atezolizumab in Patients with Untreated Metastatic Urothelial Cancer

Clinically relevant biomarkers are needed to predict outcomes with immunotherapy in metastatic urothelial carcinoma (mUC). Different PD-L1 assays use various methods (e.g., scoring tumor and/or immune cells) to evaluate anti-PD-L1/PD-1 outcomes. In the Phase III IMvigor130 study, a trend toward favorable overall survival (OS) was seen with atezolizumab monotherapy (Arm B) versus placebo+platinum-based chemotherapy (i.e., gemcitabine with either cisplatin or carboplatin) (Arm C) in first-line (1L) treatment of patients with mUC who had high PD-L1 IC expression by the SP142 IHC assay (IC2/3: HR, 0.68, 95% CI: 0.43, 1.08; IC2/3 cisplatin ineligible: HR, 0.53, 95% CI: 0.30, 0.94 (May 31, 2019 cutoff)). Thus, it was hypothesized that the SP142 and 22C3 assays may be differentially associated with OS in IMvigor130 patients, which may be related to PD-L1 localization on specific immune cells such as dendritic cells (DCs). This Example describes a post hoc analysis that studied associations between two PD-L1 IHC assays (i.e., VENTANA SP142 and Dako 22C3) and OS in the biomarker-evaluable population of the IMvigor130 study.

### A. Materials and Methods

The biomarker-evaluable population in this analysis consisted of patients with archival tumors in Arms B and C (Jun. 14, 2020 cutoff) (Galsky et al. Cancer Res. 81 (13_Supplement): CT042, 2021). Formalin-fixed paraffin-embedded (FFPE) tumor specimens from eligible IMvigor130 study patients were prospectively tested for PD-L1 IHC expression by a central laboratory prior to randomization (see, Example 1 for additional information). The IHC scores had three categories (IC0, IC1, IC2/3). The study enrolled all eligible patients whose tissue is evaluable for expression testing, regardless of PD-L1 expression status.

PD-L1 expression was evaluated by IHC (CellCarta) using the SP142 (Ventana) and 22C3 (Dako) assays in 322 patients treated with atezolizumab monotherapy (Arm B) and 305 patients treated with placebo+platinum-based chemotherapy (Arm C). Samples were also stained for IC subtypes. Assay cutoffs for the SP142 assay were IC ≥5% (IC2/3) and <5% (IC0/1). For the 22C3 assay, cutoffs were CPS ≥10 and <10.

A subset of tumor samples were also stained by IHC for the dendritic cell marker DC-LAMP (Clone 1010E1.01, Novus Biologicals). In silico deconvolution by xCell (Aran et al. Genome Biol. 18 (1): 220, 2017) was performed. Statistical analysis was performed to compare OS between subgroups using the stratified log-rank test.

### B. Results

i. SP142 IHC Assay Staining Co-Localized with Dendritic Cells Instead of Other Myeloid Subsets

To deconvolute the cellular components associate with PD-L1 staining, colocalization of SP142 and DC-LAMP staining in mUC tumor tissue was performed. The staining results showed that the SP142 IHC assay preferentially stained cells that were also positive for DC-LAMP (FIGS. 5A-5C), which is a dendritic cell-specific marker. This is consistent with data from model systems demonstrating that PD-L1 expression by DCs is a key regulator of T-cell immunity in cancer, suggesting that DCs are clinically relevant PD-L1-expressing IC. Immune cells adjacent to an invasive UC strand showed double labelling for SP142 (brown) and DC-LAMP (green) (FIG. 5C). These data indicated that SP142 PD-L1 assay staining preferentially co-localized with DCs compared with other myeloid subsets of IC.

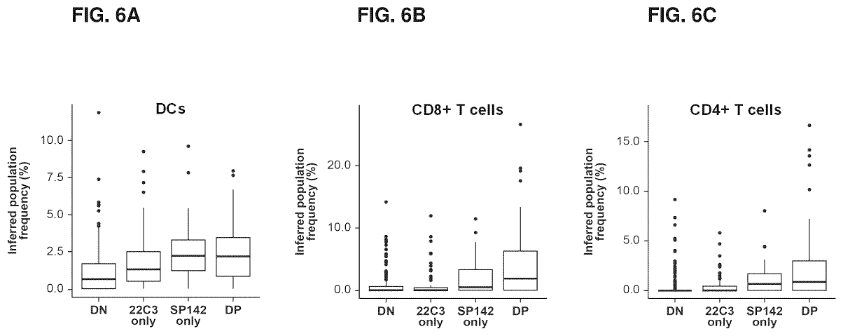

Bulk RNA-seq was conducted in 258 patients treated with atezolizumab monotherapy (Arm B) and 283 patients treated with placebo+platinum-based chemotherapy (Arm C). In silico deconvolution by xCell (Aran et al. Genome Biol. 18 (1): 220, 2017) suggested that a higher frequency of DCs and T cells occurred in SP142 IC2/3 than in IC0/1 tumors (FIGS. 6A-6C).

ii. SP142 Scoring Had Improved Prognostic Ability Than 22C3 CPS Scoring in Patients Receiving Atezolizumab Monotherapy

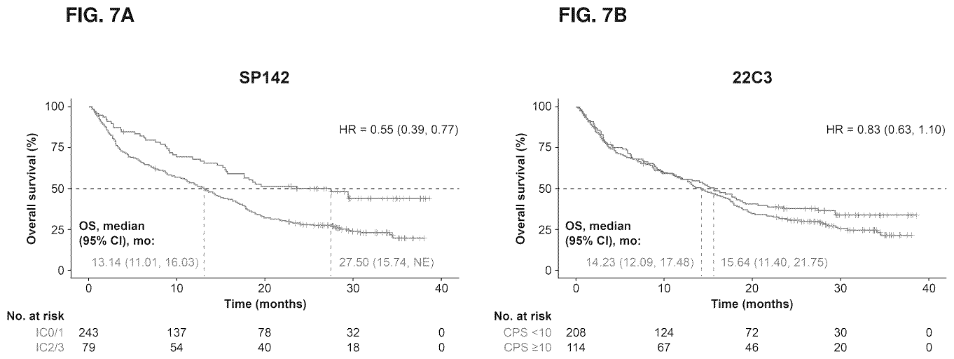

Associations between OS and the SP142 and 22C3 assays were analyzed in the biomarker-evaluable population of the IMvigor130 study. SP142 scoring (HR=0.55 (0.39, 0.77)) appeared to have improved prognostic ability than 22C3 CPS scoring (HR=0.83 (0.63, 1.10)) in patients receiving atezolizumab monotherapy (FIGS. 7A and 7B).

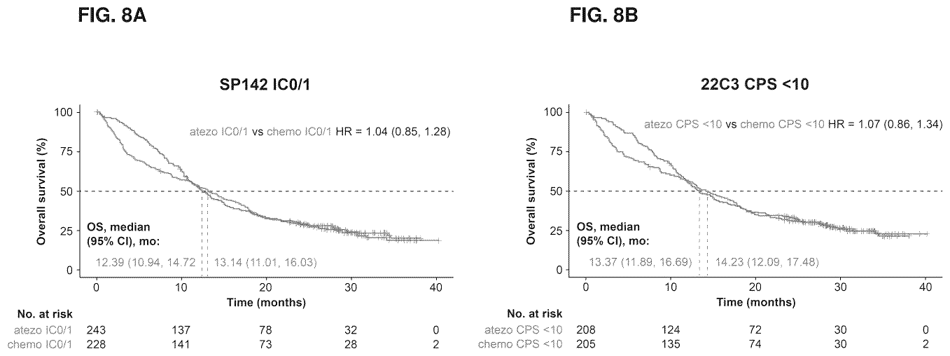

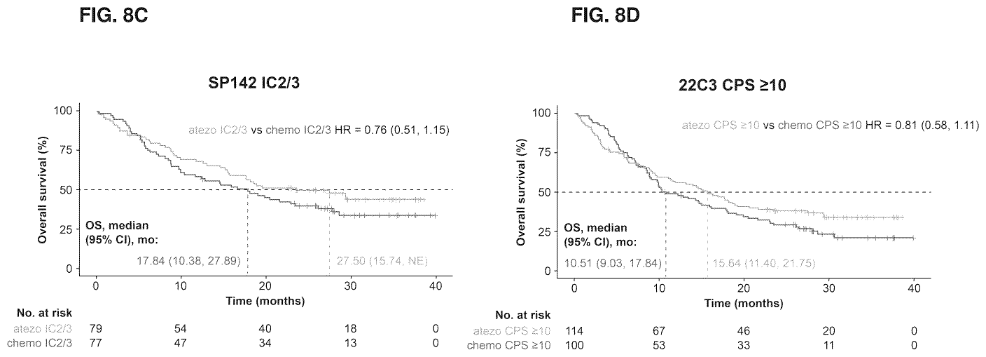

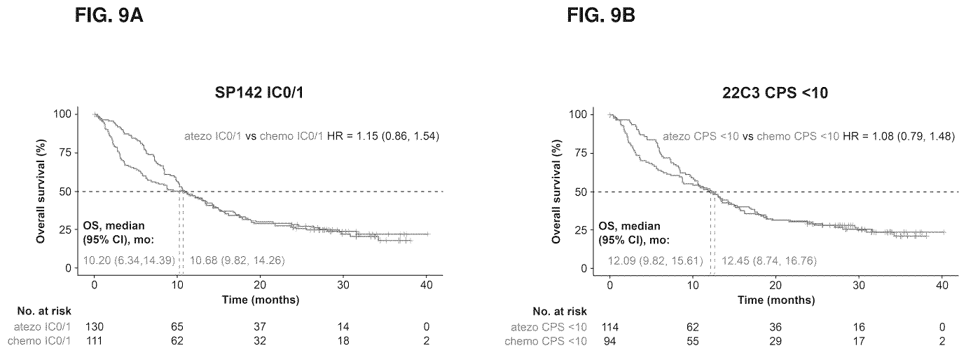

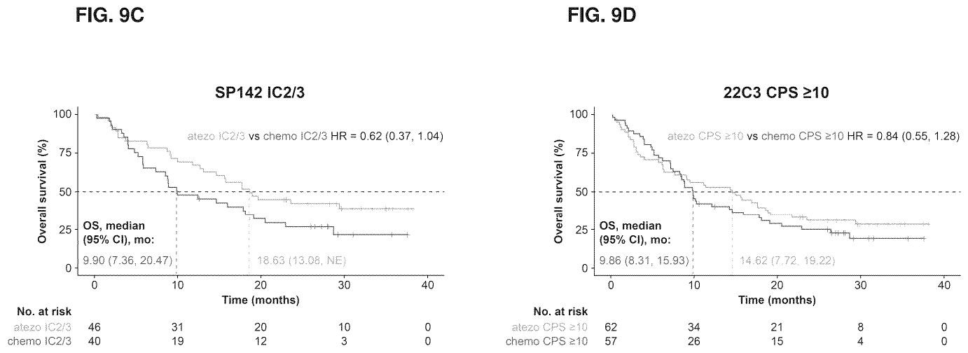

In addition, the 22C3 assay selected a larger pool of patients receiving atezolizumab monotherapy with high PD-L1 expression (CPS ≥10; 35%) than the SP142 assay (IC2/3; 25%), thus diluting the PD-L1-high population (FIGS. 8A-8D). Patients in Arms B and C who were selected by the 22C3 assay had shorter median OS than those selected by the SP142 assay. Similar results were observed in the cisplatin-ineligible population (FIGS. 9A-9D), which also demonstrated that high SP142 PD-L1 expression was associated with better prognosis and was a predictive biomarker for atezolizumab monotherapy versus platinum-based chemotherapy in patients. In summary, high SP142 PD-L1 expression was associated with improved prognosis and was a predictive biomarker for patients receiving atezolizumab monotherapy compared to chemotherapy.

iii. Combined Analysis with SP142 and 22C3 Assays Show Longer OS in Tumors staining for High PD-L1 in Both Assays

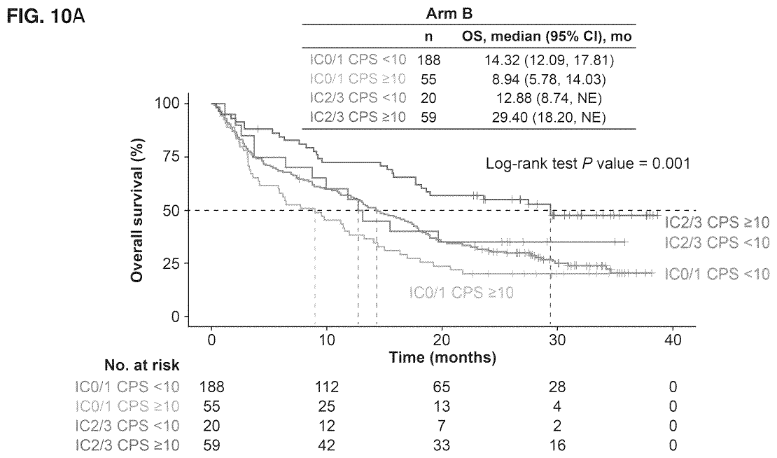

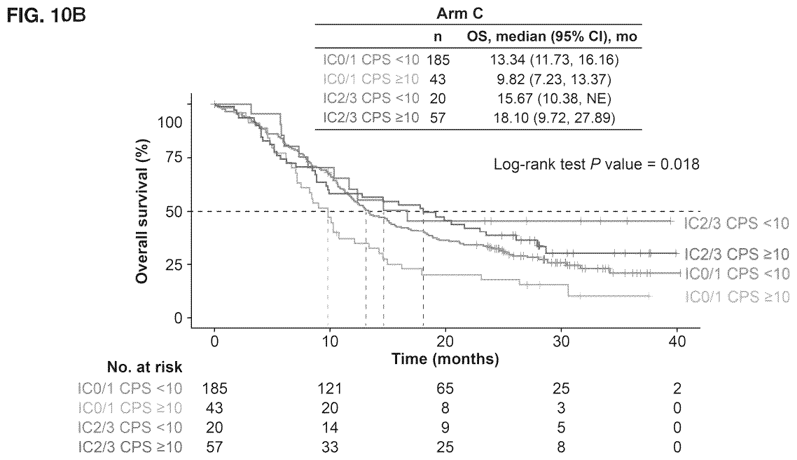

Associations between OS and the combination of the SP142 and 22C3 assays were analyzed in the biomarker-evaluable population of the IMvigor130 study (FIGS. 10A and 10B; Tables 4 and 5). Combined analysis with the SP142 and 22C3 assays showed longer OS was associated with SP142 IC2/3+22C3 CPS ≥10 (double positives) tumor status, while shortest OS was observed in patients with tumors staining for SP142 IC0/1+22C3 CPS ≥10.

TABLE 4
Subgroup Analysis of Arm B in IMvigor130

| | |
|---|---|
| Patient selection in | SP142 |

| | | | |
|---|---|---|---|
| Arm B, n (%)^{a} | IC0/1 | IC2/3 | Total |

| | | | | |
|---|---|---|---|---|
| 22C3 | CPS <10 | 188 (58) | 20 (6) | 208 (65) |
| | CPS ≥10 | 55 (17) | 59 (18) | 114 (35) |
| | Total | 243 (75) | 79 (25) | 322 (100) |

^{a}Percentages were calculated using the total number of patients in the biomarker-evaluable population of each arm.

TABLE 5
Subgroup Analysis of Arm C in IMvigor130

| | |
|---|---|
| Patient selection in | SP142 |

| | | | |
|---|---|---|---|
| Arm C, n (%)^{a} | IC0/1 | IC2/3 | Total |

| | | | | |
|---|---|---|---|---|
| 22C3 | CPS <10 | 185 (61) | 20 (7) | 205 (67) |
| | CPS ≥10 | 43 (14) | 57 (19) | 100 (33) |
| | Total | 228 (75) | 77 (25) | 305 (100) |

^{a}Percentages were calculated using the total number of patients in the biomarker-evaluable population of each arm.

Therefore, longer OS associated with tumors staining for high PD-L1 expression by both SP142 and 22C3 assays. Shortest OS was observed with tumors staining for high PD-L1 by the 22C3 assay but not the SP142 assay.

### C. Conclusions

This post hoc analysis of the IMvigor130 study demonstrated the predictive and prognostic value of the SP142 and 22C3 assays for OS in patients with mUC. SP142 PD-L1 staining preferentially co-localized with DCs compared with other myeloid subsets of IC. In the biomarker-evaluable population, the SP142 assay was found to have both prognostic and predictive ability. The SP142 assay was associated with OS while the 22C3 assay was not associated. Combined analysis with the SP142 and 22C3 assays showed longer OS associated with tumors staining for high PD-L1 expression by both SP142 and 22C3 assays. The shortest OS was shown with tumors staining for high PD-L1 by the 22C3, but not the SP142 assay. In conclusion, PD-L1-expressing DCs may explain the longer OS with atezolizumab in SP142 IC2/3 versus IC0/1 tumours in patients with mUC. These findings reinforce preclinical findings, demonstrating the particular importance of PD-L1-expressing DCs. Additionally, these data indicate that using immune-directed PD-L1 assays (e.g., using SP142) and immune-agnostic PD-L1 assays (e.g., using 22C3, SP263, or 28-8) can be used together to identify patients who are likely to benefit (e.g., in terms of OS) from treatment with PD-1 axis binding antagonists such as atezolizumab.

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1-2. (canceled)

3. A method of treating a cancer in a patient, the method comprising administering to the patient a treatment regimen comprising a PD-1 axis binding antagonist, wherein a tumor sample obtained from the patient has been determined to have:
(i) a first score obtained from applying a first scoring algorithm to a first stained portion of the tumor sample that meets or exceeds a first cutoff, wherein the first stained portion of the tumor sample was stained with an immune-directed PD-L1 assay; and
(ii) a second score obtained from applying a second scoring algorithm to a second stained portion of the tumor sample that meets or exceeds a second cutoff, wherein the second stained portion of the tumor sample was stained with an immune-agnostic PD-L1 assay.

4. The method of claim 3, wherein:
(a) the immune-directed PD-L1 assay has:
(i) at least an 80%, at least an 85%, at least a 90%, or at least a 95% overall percent agreement (OPA) with an SP142 Assay using the first scoring algorithm at the first cutoff value;
(ii) at least an 80%, at least an 85%, at least a 90%, or at least a 95% positive percent agreement (PPA) with an SP142 Assay using the first scoring algorithm at the first cutoff value;
(iii) at least an 80%, at least an 85%, at least a 90%, or at least a 95% negative percent agreement (NPA) with an SP142 Assay using the first scoring algorithm at the first cutoff value;
(iv) at least an 80%, at least an 85%, at least a 90%, or at least a 95% PPA and at least an 80%, at least an 85%, at least a 90%, or at least a 95% NPA with an SP142 Assay using the first scoring algorithm at the first cutoff value;
(v) at least an 80%, at least an 85%, at least a 90%, or at least a 95% PPA and at least an 80%, at least an 85%, at least a 90%, or at least a 95% OPA with an SP142 Assay using the first scoring algorithm at the first cutoff value;
(vi) at least an 80%, at least an 85%, at least a 90%, or at least a 95% NPA and at least an 80%, at least an 85%, at least a 90%, or at least a 95% OPA with an SP142 Assay using the first scoring algorithm at the first cutoff value; and/or
(vii) at least an 80%, at least an 85%, at least a 90%, or at least a 95% OPA, at least an 80%, at least an 85%, at least a 90%, or at least a 95% PPA, and at least an 80%, at least an 85%, at least a 90%, or at least a 95% NPA with an SP142 Assay using the first scoring algorithm at the first cutoff value; and/or
(b) the immune-agnostic PD-L1 assay has:
(i) at least an 80%, at least an 85%, at least a 90%, or at least a 95% OPA with an 22C3 Assay using the second scoring algorithm at the second cutoff value;
(ii) at least an 80%, at least an 85%, at least a 90%, or at least a 95% PPA with an 22C3 Assay using the second scoring algorithm at the second cutoff value;
(iii) at least an 80%, at least an 85%, at least a 90%, or at least a 95% NPA with an 22C3 Assay using the second scoring algorithm at the second cutoff value;
(iv) at least an 80%, at least an 85%, at least a 90%, or at least a 95% PPA and at least an 80%, at least an 85%, at least a 90%, or at least a 95% NPA with an 22C3 Assay using the second scoring algorithm at the second cutoff value;
(v) at least an 80%, at least an 85%, at least a 90%, or at least a 95% PPA and at least an 80%, at least an 85%, at least a 90%, or at least a 95% OPA with an 22C3 Assay using the second scoring algorithm at the second cutoff value;
(vi) at least an 80%, at least an 85%, at least a 90%, or at least a 95% NPA and at least an 80%, at least an 85%, at least a 90%, or at least a 95% OPA with an 22C3 Assay using the second scoring algorithm at the second cutoff value; and/or
(vii) at least an 80%, at least an 85%, at least a 90%, or at least a 95% OPA, at least an 80%, at least an 85%, at least a 90%, or at least a 95% PPA, and at least an 80%, at least an 85%, at least a 90%, or at least a 95% NPA with an 22C3 Assay using the second scoring algorithm at the second cutoff value.

5. (canceled)

6. A method of stratifying a tumor having a score with an immune-agnostic PD-L1 assay that exceeds a pre-determined cutoff, the method comprising:
(a) staining a portion of the tumor with an immune-directed PD-L1 assay to obtain a stained sample;
(b) generating an immune-directed PD-L1 assay score by applying a scoring algorithm to the stained sample; and
(c) comparing the immune-directed PD-L1 assay score to a first cutoff, wherein the tumor is likely to respond to a PD-1 axis binding antagonist when the immune-directed PD-L1 assay score meets or exceeds the first cutoff.

7. The method of claim 6, wherein the score of the tumor is a Combined Positive Score (CPS)>10% tumor, and/or wherein the immune-agnostic PD-L1 assay is a 22C3 assay, an SP263 assay, or a 28-8 assay.

8-13. (canceled)

14. The method of claim 3, wherein:
(a) the immune-directed PD-L1 assay comprises a PD-L1 immunohistochemical (IHC) assay comprising the VENTANA SP142 anti-PD-L1 diagnostic antibody; and/or
(b) the immune-agnostic PD-L1 assay comprises a PD-L1 IHC assay comprising the Dako 22C3 anti-PD-L1 diagnostic antibody, the VENTANA SP263 anti-PD-L1 diagnostic antibody, or the 28-8 anti-PD-L1 diagnostic antibody.

15. (canceled)

16. The method of claim 3, wherein:
(a) the first cutoff is a detectable expression level of PD-L1 in tumor-infiltrating immune cells that comprise 5% or more of the tumor sample; and/or
(b) the second cutoff is a CPS ≥10%.

17. (canceled)

18. A method of labeling PD-L1 in a tumor sample, the method comprising the following steps:
(a) contacting the tumor sample with the VENTANA SP142 anti-PD-L1 diagnostic antibody;
(b) contacting the tumor sample with the Dako 22C3 anti-PD-L1 diagnostic antibody, the VENTANA SP263 anti-PD-L1 diagnostic antibody, or the 28-8 anti-PD-L1 diagnostic antibody; and
(c) visualizing the anti-PD-L1 diagnostic antibodies of steps (a) and (b) with one or more detectable reagents that generates a detectable signal for both of the anti-PD-L1 diagnostic antibodies.

19. The method of claim 18, wherein;
(a) the detectable signal for the VENTANA SP142 anti-PD-L1 diagnostic antibody is an amplified signal;
(b) steps (a) and (b) are performed simultaneously or sequentially;
(c) steps (a) and (b) are performed in different sections or in the same section of the tumor sample;
(d) the visualizing comprises IHC or immunofluorescence (IF); and/or
(e) the tumor sample is obtained from a patient having a cancer.

20-28. (canceled)

29. The method of claim 3, wherein:
(a) the tumor sample is an FFPE tumor sample, an archival tumor sample, a fresh tumor sample, or a frozen tumor sample; and/or
(b) the cancer is a bladder cancer, a kidney cancer, a lung cancer, a cancer of the urinary tract, a breast cancer, a prostate cancer, a cancer of the peritoneum, a hepatocellular cancer, a gastric or stomach cancer, a pancreatic cancer, a glioblastoma, a cervical cancer, an ovarian cancer, a liver cancer, a hepatoma, a colon cancer, a rectal cancer, a colorectal cancer, an endometrial or uterine carcinoma, a salivary gland carcinoma, a prostate cancer, a vulval cancer, a thyroid cancer, a hepatic carcinoma, an anal carcinoma, a penile carcinoma, a melanoma, a multiple myeloma or B-cell lymphoma, a chronic lymphocytic leukemia (CLL), an acute lymphoblastic leukemia (ALL), an acute myologenous leukemia (AML), a hairy cell leukemia, a chronic myeloblastic leukemia (CML), a post-transplant lymphoproliferative disorder (PTLD), a myelodysplastic syndrome (MDS), Meigs' syndrome, a brain cancer, or a head and neck cancer.

30. (canceled)

31. The method of claim 29, wherein the cancer is a bladder cancer, and wherein the bladder cancer is a urothelial carcinoma (UC).

32. (canceled)

33. The method of claim 31, wherein the UC is a locally advanced or metastatic UC, and wherein:
(a) the patient is previously untreated for the locally advanced or metastatic UC; and/or
(b) the locally advanced or metastatic UC is histologically documented, locally advanced (T4b, any N; or any T, N2-3) or metastatic urothelial carcinoma (mUC) (M1, Stage IV).

34-42. (canceled)

43. The method of claim 19, wherein the method identifies the patient as one who may benefit from a treatment regimen comprising a PD-1 axis binding antagonist.

44. (canceled)

45. The method of claim 3, wherein:
(a) the PD-1 axis binding antagonist is a PD-L1 binding antagonist, a PD-1 binding antagonist, or a PD-L2 binding antagonist; and/or
(b) the PD-1 axis binding antagonist is administered to the patient as a monotherapy or in combination with one or more additional therapeutic agents.

46. (canceled)

47. The method of claim 45, wherein the PD-1 axis binding antagonist is an anti-PD-L1 antibody, and wherein the anti-PD-L1 antibody comprises the following HVRs:
(a) an HVR-H1 sequence of GFTFSDSWIH (SEQ ID NO: 3);
(b) an HVR-H2 sequence of AWISPYGGSTYYADSVKG (SEQ ID NO: 4);
(c) an HVR-H3 sequence of RHWPGGFDY (SEQ ID NO: 5);
(d) an HVR-L1 sequence of RASQDVSTAVA (SEQ ID NO: 6);
(e) an HVR-L2 sequence of SASFLYS (SEQ ID NO: 7); and
(f) an HVR-L3 sequence of QQYLYHPAT (SEQ ID NO: 8).

48. (canceled)

49. The method of claim 47, or 48, wherein:
(a) the anti-PD-L1 antibody comprises:
(i) a heavy chain comprising the amino acid sequence of SEQ ID NO: 1; and/or
(ii) a light chain comprising the amino acid sequence of SEQ ID NO: 2; or
(b) the anti-PD-L1 antibody is atezolizumab.

50. (canceled)

51. The method of claim 49, wherein atezolizumab is administered to the patient intravenously at a dose of about 840 mg every 2 weeks, about 1200 mg every 3 weeks, or about 1680 mg every 4 weeks.

52. The method of claim 51, wherein atezolizumab is administered to the patient intravenously at a dose of about 1200 mg every 3 weeks in 21-day dosing cycles, and wherein atezolizumab is administered to the patient intravenously at a dose of about 1200 mg on Day-2 to Day 4 of each 21-day dosing cycle or on Day 1 of each 21-day dosing cycle.

53-56. (canceled)

57. The method of claim 45, wherein the one or more additional therapeutic agents comprise a platinum-based chemotherapy.

58. The method of claim 57, wherein the platinum-based chemotherapy comprises a platinum-based chemotherapeutic agent and a nucleoside analog.

59. The method of claim 58, wherein:
(a) the platinum-based chemotherapeutic agent is cisplatin, carboplatin, or oxaliplatin; and/or
(b) the nucleoside analog is gemcitabine.

60-65. (canceled)

66. The method of claim 43, wherein the benefit from the treatment regimen comprising the PD-1 axis binding antagonist is in terms of overall survival (OS).

67. The method of claim 66, wherein:
(a) the treatment regimen extends the patient's OS by from about 5.7 months to about 17 months as compared to treatment with a platinum-based chemotherapy without the PD-1 axis binding antagonist; or
(b) the treatment regimen extends the patient's OS by about 11.3 months as compared to treatment with a platinum-based chemotherapy without the PD-1 axis binding antagonist.

68-69. (canceled)

70. The method of claim 3, wherein the tumor sample obtained from the patient has:
(a) the presence of discernible PD-L1 staining of any intensity in tumor-infiltrating immune cells covering ≥5% of tumor area occupied by tumor cells, associated intratumoral, and contiguous peritumoral stroma, as determined by the PD-L1 IHC assay comprising the VENTANA SP142 anti-PD-L1 diagnostic antibody; or
(b) a CPS of ≥10 using a PD-L1 IHC assay comprising:
(i) the Dako 22C3 anti-PD-L1 diagnostic antibody;
(ii) the VENTANA SP263 anti-PD-L1 diagnostic antibody; or
(iii) the 28-8 anti-PD-L1 diagnostic antibody.

71-87. (canceled)

88. A kit comprising:
(a) a VENTANA SP142 anti-PD-L1 diagnostic antibody; and
(b) a Dako 22C3 anti-PD-L1 diagnostic antibody, a VENTANA SP263 anti-PD-L1 diagnostic antibody, or a 28-8 anti-PD-L1 diagnostic antibody.

89. (canceled)
